SEQUENCE LISTING (32 sequences)

SEQ ID NO: 1 - DNA, 946 bp, unidentified
  source 1..946: mol_type = other DNA; note = human papillomavirus type 16
aaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaaactacaataattcatgtataaaactaagggcgtaaccgaaatcggttgaaccgaaaccggttagtataaaagcagacattttatgcaccaaaagagaact

SEQ ID NO: 2 - DNA, 736 bp, unidentified
  source 1..736: mol_type = other DNA; note = human papillomavirus type 16
gtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaaactacaataattcatgtataaaactaagggcgtaaccgaaatcggttgaaccgaaaccggttagtataaaagcagacattttatgcaccaaaagagaact

SEQ ID NO: 3 - DNA, 358 bp, unidentified
  source 1..358: mol_type = other DNA; note = human papillomavirus type 16
tgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaaactacaataattcatgtataaaactaagggcgtaaccgaaatcggttgaaccgaaaccggttagtataaaagcagacattttatgcaccaaaagagaact

SEQ ID NO: 4 - DNA, 276 bp, unidentified
  source 1..276: mol_type = other DNA; note = human papillomavirus type 16
ctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaaactacaataattcatgtataaaactaagggcgtaaccgaaatcggttgaaccgaaaccggttagtataaaagcagacattttatgcaccaaaagagaact

SEQ ID NO: 5 - DNA, 194 bp, unidentified
  source 1..194: mol_type = other DNA; note = human papillomavirus type 16
tagtcatacattgttcatttgtaaaactgcacatgggtgtgtgcaaaccgattttgggttacacatttacaagcaacttatataataatactaaactacaataattcatgtataaaactaagggcgtaaccgaaatcggttgaaccgaaaccggttagtataaaagcagacattttatgcaccaaaagagaact

SEQ ID NO: 6 - DNA, 1950 bp, unidentified
  source 1..1950: mol_type = other DNA; note = human papillomavirus type 16
atggcagaccccgctggaacaaatggagaggagggcactgggtgtaacggctggttttacgtggaagcagtcgtagagaagaagacaggcgacgccatttcagacgacgagaatgagaacgatagcgacactggtgaggatcttgtggactttattgtgaacgacaatgactatctcacccaggcagaaaccgagaccgcccacgccctcttcacagcccaggaagctaagcaacatcgggatgcagtgcaggtgctcaaaagaaagtacctggttagtcctctgtccgacatctctggatgcgtcgacaataatatcagtccaaggctgaaggctatatgcatagagaagcagtcaagagcggcgaagaggagactgtttgaaagcgaggatagtggatacgggaacacagaagtcgagacccaacagatgctccaggtggagggtcgccatgagactgagaccccctgctcccagtacagcggcggatcaggcggtggatgctctcagtactccagtgggtccggcggggagggtgtttccgaaagacacaccatctgtcagacccccctgactaatattctgaacgtactgaaaacatccaacgccaaggctgccatgctggcgaagtttaaggagctgtatggcgtgagcttcagcgaactggtgagaccattcaagagcaacaagagcacctgttgtgattggtgtattgccgcctttgggctgactccatccatcgctgactctattaaaaccctgttgcaacagtactgcctctacctgcatattcagtccctcgcttgctcctggggaatggtggtgctgcttctggttcggtataagtgtggcaaaaacagggagaccatcgagaagctccttagtaagctcctgtgtgtgtctcccatgtgcatgatgattgaaccgccaaaattgcggagcacggccgccgccctgtactggtacaaaacaggcataagcaacatcagcgaagtgtatggtgacacgccagaatggatacagagacagaccgtgctccagcacagttttaacgattgcacatttgagctgtctcagatggtgcagtgggcttatgataatgacattgtagacgattccgaaatagcgtataagtacgcccagctcgcagataccaattccaatgccagcgcatttctgaagtccaattcacaggcaaagatagtaaaggattgcgctacaatgtgccgccattataaaagagcggagaaaaagcagatgtcaatgtcccaatggatcaagtataggtgtgatcgcgttgatgatggcggtgattggaagcagatcgtgatgttcctccgctatcaaggcgtagaattcatgtcattcctgaccgccctgaaacgcttcctgcagggcattcctaaaaaaaattgcatcctgctgtatggcgcggctaacactggaaagagtctgttcggcatgagccttatgaagttcctccagggatccgtgatatgctttgtgaacagcaaatcacacttttggcttcagccattggcagatgcaaagatcggcatgctggacgacgccacagtcccatgctggaactacatagacgataatctccgaaacgcattggacggcaatctggtgagcatggacgtcaagcacaggcctctggtgcaactgaagtgtccccctctcctcattacgtcaaacatcaacgccggaacagatagtcggtggccgtacctgcacaatagacttgtggtgtttacatttcctaatgaattcccatttgacgaaaacggcaatccagtatacgagctgaatgacaagaactggaagagttttttctctaggacatggtccaggttgagtctccacgaagacgaggataaagagaatgacggagactctttgcccacttttaagtgcgtgtctggacaaaataccaataccctgtga

SEQ ID NO: 7 - DNA, 1098 bp, unidentified
  source 1..1098: mol_type = other DNA; note = human papillomavirus type 16
atggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 8 - DNA, 1443 bp, unidentified
  source 1..1443: mol_type = other DNA; note = human papillomavirus type 16
atgtactccagtgggtccggcggggagggtgtttccgaaagacacaccatctgtcagacccccctgactaatattctgaacgtactgaaaacatccaacgccaaggctgccatgctggcgaagtttaaggagctgtatggcgtgagcttcagcgaactggtgagaccattcaagagcaacaagagcacctgttgtgattggtgtattgccgcctttgggctgactccatccatcgctgactctattaaaaccctgttgcaacagtactgcctctacctgcatattcagtccctcgcttgctcctggggaatggtggtgctgcttctggttcggtataagtgtggcaaaaacagggagaccatcgagaagctccttagtaagctcctgtgtgtgtctcccatgtgcatgatgattgaaccgccaaaattgcggagcacggccgccgccctgtactggtacaaaacaggcataagcaacatcagcgaagtgtatggtgacacgccagaatggatacagagacagaccgtgctccagcacagttttaacgattgcacatttgagctgtctcagatggtgcagtgggcttatgataatgacattgtagacgattccgaaatagcgtataagtacgcccagctcgcagataccaattccaatgccagcgcatttctgaagtccaattcacaggcaaagatagtaaaggattgcgctacaatgtgccgccattataaaagagcggagaaaaagcagatgtcaatgtcccaatggatcaagtataggtgtgatcgcgttgatgatggcggtgattggaagcagatcgtgatgttcctccgctatcaaggcgtagaattcatgtcattcctgaccgccctgaaacgcttcctgcagggcattcctaaaaaaaattgcatcctgctgtatggcgcggctaacactggaaagagtctgttcggcatgagccttatgaagttcctccagggatccgtgatatgctttgtgaacagcaaatcacacttttggcttcagccattggcagatgcaaagatcggcatgctggacgacgccacagtcccatgctggaactacatagacgataatctccgaaacgcattggacggcaatctggtgagcatggacgtcaagcacaggcctctggtgcaactgaagtgtccccctctcctcattacgtcaaacatcaacgccggaacagatagtcggtggccgtacctgcacaatagacttgtggtgtttacatttcctaatgaattcccatttgacgaaaacggcaatccagtatacgagctgaatgacaagaactggaagagttttttctctaggacatggtccaggttgagtctccacgaagacgaggataaagagaatgacggagactctttgcccacttttaagtgcgtgtctggacaaaataccaataccctgtga

SEQ ID NO: 9 - DNA, 1104 bp, unidentified
  source 1..1104: mol_type = other DNA; note = human papillomavirus type 11
atggaagccattgccaaaaggcttgatgcttgccaggatcagcttctcgagctgtatgaggagaactctattgacattcataaacacatcatgcactggaaatgcattagactggagagcgtgttgctgcacaaagcgaagcagatgggactgagccacattgggcttcaggtggtcccaccccttactgtgtcagagacaaaggggcataatgccatcgagatgcagatgcatttggagtccctggcgaaaacccagtatggtgtcgagccatggacgctgcaggacaccagttacgaaatgtggctcaccccacccaaacgctgctttaagaagcagggaaatactgtggaggtaaagttcgatggctgtgaggacaatgttatggagtacgtggtctggacacacatctacttgcaggataatgactcttgggtaaaagtcacttcctccgttgatgccaagggcatctattacacgtgtggacaattcaagacgtactacgtcaatttcaataaggaagctcagaagtacggcagcacaaaccattgggaagtttgctatggctctactgttatttgttcccctgcttcagtgagtagcacagtccgggaagtcagtatagccgaacccaccacttacaccccagcccagacaaccgcccctacagtttccgcttgcaccactgaggacggcgtgtctgcacctccccgcaagcgtgcaagaggacccagcactaacaacaccctgtgtgtggccaacatacggtcagtggacagtacaatcaacaacatcgtaaccgacaattacaacaagcaccagaggcggaataattgtcactccgcagcaacaccgatagtgcaactgcaaggtgatagcaactgcctgaaatgcttccgctataggctgaatgataagtataaacacctgtttgaactggcatctagcacctggcattgggcctctcctgaagctccacacaagaacgctattgtgacactgacttatagctccgaagagcaacgacagcaatttctgaacagcgtgaaaatccctccgaccatcagacataaggtggggtttatgtcactccatctcctctaa

SEQ ID NO: 10 - DNA, 3108 bp, unidentified
  source 1..3108: mol_type = other DNA; note = human papillomavirus type 16
atggcagaccccgctggaacaaatggagaggagggcactgggtgtaacggctggttttacgtggaagcagtcgtagagaagaagacaggcgacgccatttcagacgacgagaatgagaacgatagcgacactggtgaggatcttgtggactttattgtgaacgacaatgactatctcacccaggcagaaaccgagaccgcccacgccctcttcacagcccaggaagctaagcaacatcgggatgcagtgcaggtgctcaaaagaaagtacctggttagtcctctgtccgacatctctggatgcgtcgacaataatatcagtccaaggctgaaggctatatgcatagagaagcagtcaagagcggcgaagaggagactgtttgaaagcgaggatagtggatacgggaacacagaagtcgagacccaacagatgctccaggtggagggtcgccatgagactgagaccccctgctcccagtacagcggcggatcaggcggtggatgctctcagtactccagtgggtccggcggggagggtgtttccgaaagacacaccatctgtcagacccccctgactaatattctgaacgtactgaaaacatccaacgccaaggctgccatgctggcgaagtttaaggagctgtatggcgtgagcttcagcgaactggtgagaccattcaagagcaacaagagcacctgttgtgattggtgtattgccgcctttgggctgactccatccatcgctgactctattaaaaccctgttgcaacagtactgcctctacctgcatattcagtccctcgcttgctcctggggaatggtggtgctgcttctggttcggtataagtgtggcaaaaacagggagaccatcgagaagctccttagtaagctcctgtgtgtgtctcccatgtgcatgatgattgaaccgccaaaattgcggagcacggccgccgccctgtactggtacaaaacaggcataagcaacatcagcgaagtgtatggtgacacgccagaatggatacagagacagaccgtgctccagcacagttttaacgattgcacatttgagctgtctcagatggtgcagtgggcttatgataatgacattgtagacgattccgaaatagcgtataagtacgcccagctcgcagataccaattccaatgccagcgcatttctgaagtccaattcacaggcaaagatagtaaaggattgcgctacaatgtgccgccattataaaagagcggagaaaaagcagatgtcaatgtcccaatggatcaagtataggtgtgatcgcgttgatgatggcggtgattggaagcagatcgtgatgttcctccgctatcaaggcgtagaattcatgtcattcctgaccgccctgaaacgcttcctgcagggcattcctaaaaaaaattgcatcctgctgtatggcgcggctaacactggaaagagtctgttcggcatgagccttatgaagttcctccagggatccgtgatatgctttgtgaacagcaaatcacacttttggcttcagccattggcagatgcaaagatcggcatgctggacgacgccacagtcccatgctggaactacatagacgataatctccgaaacgcattggacggcaatctggtgagcatggacgtcaagcacaggcctctggtgcaactgaagtgtccccctctcctcattacgtcaaacatcaacgccggaacagatagtcggtggccgtacctgcacaatagacttgtggtgtttacatttcctaatgaattcccatttgacgaaaacggcaatccagtatacgagctgaatgacaagaactggaagagttttttctctaggacatggtccaggttgagtctccacgaagacgaggataaagagaatgacggagactctttgcccacttttaagtgcgtgtctggacaaaataccaataccctgggaagcggagagggcagaggaagtctgctaacatgcggtgacgtcgaggagaatcctggacctatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccactccgccgcgacccataccaaagccgtcgccttgggcaccgaagaaacacagacgactatgcagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 11 - DNA, 20 bp, synthetic construct
  misc_feature 1..20: note = Primer
  source 1..20: mol_type = other DNA
ctaattcactcccaacgaag

SEQ ID NO: 12 - DNA, 20 bp, synthetic construct
  misc_feature 1..20: note = Primer
  source 1..20: mol_type = other DNA
gccgagtcctgcgtcgagag

SEQ ID NO: 13 - DNA, 2187 bp, synthetic construct
  misc_feature 1..2187: note = Anti-HER2 Antibody
  source 1..2187: mol_type = other DNA
atgtacaggatgcaactcctgtcttgcattgcactaagtcttgcacttgtcacggaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgtacaggatgcaactcctgtcttgcattgcactaagtcttgcacttgtcacggatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctcgagtctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - DNA, 2085 bp, synthetic construct
  misc_feature 1..2085: note = Anti-EGFR Antibody
  source 1..2085: mol_type = other DNA
atgcaggtgcagctgaagcagagcggcccggggctcgtccagccctcgcagagcctgagcatcacctgcacggtgagcggcttcagcctgaccaactacggggtgcactgggtccggcagtcgcccggcaaggggctggagtggctgggcgtgatctggagcggcgggaacaccgactacaacacccccttcacgagccgcctgagcatcaacaaggacaacagcaagtcgcaggtgttcttcaagatgaacagcctccagagcaacgacaccgccatctactactgcgcgcgggccctgacctactacgactacgagttcgcctactggggccaggggaccctggtcacggtgagcgccgcgagcaccaagggcccgagcgtgttccccctcgccccctccagcaagagcaccagcggcgggaccgccgccctgggctgcctggtcaaggactacttccccgagccggtgacggtgagctggaactcgggggccctcaccagcggcgtccacaccttccccgcggtgctgcagagcagcgggctgtacagcctcagctcggtggtcaccgtgcccagcagcagcctgggcacgcagacctacatctgcaacgtgaaccacaagcccagcaacaccaaggtcgacaagcgcgtggagccgaagtcgcccaagagctgcgacaagacccacacgtgcccgccctgccccgcccccgagctgctcggcgggcccagcgtgttcctgttcccgcccaagcccaaggacaccctgatgatcagccggacccccgaggtcacctgcgtggtggtcgacgtgagccacgaggacccggaggtgaagttcaactggtacgtcgacggcgtggaggtgcacaacgccaagacgaagccccgcgaggagcagtacaacagcacctaccgggtcgtgtcggtgctcaccgtcctgcaccaggactggctgaacgggaaggagtacaagtgcaaggtgagcaacaaggccctccccgcgcccatcgagaagaccatcagcaaggccaagggccagccgcgcgagccccaggtgtacacgctgccccccagccgggacgagctgaccaagaaccaggtcagcctcacctgcctggtgaaggggttctacccgtcggacatcgccgtggagtgggagagcaacggccagcccgagaacaactacaagaccacgcccccggtcctggacagcgacggcagcttcttcctctacagcaagctgaccgtggacaagagccgctggcagcaggggaacgtgttctcgtgcagcgtcatgcacgaggccctgcacaaccactacacccagaagagcctcagcctgagccccggcaagtgaggaagcggagagggcagaggaagtctgctaacatgcggtgacgtcgaggagaatcctggacctggatccatggacatcctgctcacccagagcccggtgatcctgtcggtcagccccggcgagcgggtgagcttcagctgccgcgccagccagtcgatcgggacgaacatccactggtaccagcagcggaccaacggcagcccccgcctgctcatcaagtacgcgagcgagagcatcagcgggattccctcgcggttcagcggcagcgggagcggcaccgacttcaccctgagcatcaacagcgtggagtcggaggacatcgccgactactactgccagcagaacaacaactggccgacgaccttcggcgccgggaccaagctggagctcaagcgcgaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcg

SEQ ID NO: 15 - DNA, 18 bp, synthetic construct
  misc_feature 1..18: note = microRNA sequence against CCR5
  source 1..18: mol_type = other DNA
gagcaagctcagtttaca

SEQ ID NO: 16 - DNA, 1687 bp, unidentified
  source 1..1687: mol_type = other DNA; note = Epstein-Barr Virus
atgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggt

SEQ ID NO: 17 - DNA, 726 bp, Homo sapiens
  source 1..726: mol_type = other DNA
atgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctag

SEQ ID NO: 18 - DNA, 2193 bp, Homo sapiens
  source 1..2193: mol_type = other DNA
atgcccggcgtggcccgcctgccgctgctgctcgggctgctgctgctcccgcgtcccggccggccgctggacttggccgactacacctatgacctggcggaggaggacgactcggagcccctcaactacaaagacccctgcaaggcggctgcctttcttggggacattgccctggacgaagaggacctgagggccttccaggtacagcaggctgtggatctcagacggcacacagctcgtaagtcctccatcaaagctgcagttccaggaaacacttctacccccagctgccagagcaccaacgggcagcctcagaggggagcctgtgggagatggagaggtagatcccgtagccggcgggcggcgacgtcccgaccagagcgtgtgtggcccgatggggtcatcccctttgtcattgggggaaacttcactggtagccagagggcagtcttccggcaggccatgaggcactgggagaagcacacctgtgtcaccttcctggagcgcactgacgaggacagctatattgtgttcacctatcgaccttgcgggtgctgctcctacgtgggtcgccgcggcgggggcccccaggccatctccatcggcaagaactgtgacaagttcggcattgtggtccacgagctgggccacgtcgtcggcttctggcacgaacacactcggccagaccgggaccgccacgtttccatcgttcgtgagaacatccagccagggcaggagtataacttcctgaagatggagcctcaggaggtggagtccctgggggagacctatgacttcgacagcatcatgcattacgctcggaacacattctccaggggcatcttcctggataccattgtccccaagtatgaggtgaacggggtgaaacctcccattggccaaaggacacggctcagcaagggggacattgcccaagcccgcaagctttacaagtgcccagcctgtggagagaccctgcaagacagcacaggcaacttctcctcccctgaataccccaatggctactctgctcacatgcactgcgtgtggcgcatctctgtcacacccggggagaagatcatcctgaacttcacgtccctggacctgtaccgcagccgcctgtgctggtacgactatgtggaggtccgagatggcttctggaggaaggcgcccctccgaggccgcttctgcgggtccaaactccctgagcctatcgtctccactgacagccgcctctgggttgaattccgcagcagcagcaattgggttggaaagggcttctttgcagtctacgaagccatctgcgggggtgatgtgaaaaaggactatggccacattcaatcgcccaactacccagacgattaccggcccagcaaagtctgcatctggcggatccaggtgtctgagggcttccacgtgggcctcacattccagtcctttgagattgagcgccacgacagctgtgcctacgactatctggaggtgcgcgacgggcacagtgagagcagcaccctcatcgggcgctactgtggctatgagaagcctgatgacatcaagagcacgtccagccgcctctggctcaagttcgtctctgacgggtccattaacaaagcgggctttgccgtcaactttttcaaagaggtggacgagtgctctcggcccaaccgcgggggctgtgagcagcggtgcctcaacaccctgggcagctacaagtgcagctgtgaccccgggtacgagctggccccagacaagcgccgctgtgaggctgcttgtggcggattcctcaccaagctcaacggctccatcaccagcccgggctggcccaaggagtacccccccaacaagaactgcatctggcagctggtggcccccacccagtaccgcatctccctgcagtttgacttctttgagacagagggcaatgatgtgtgcaagtacgacttcgtggaggtgcgcagtggactcacagctgactccaagctgcatggcaagttctgtggttctgagaagcccgaggtcatcacctcccagtacaacaacatgcgcgtggagttcaagtccgacaacaccgtgtccaaaaagggcttcaaggcccacttcttctcagaaaagaggccagctctgcagccccctcggggacgcccccaccagctcaaattccgagtgcagaaaagaaaccggaccccccagtga

SEQ ID NO: 19 - DNA, 7905 bp, unidentified
  source 1..7905: mol_type = other DNA; note = human papillomavirus type 16
actacaataatccatgtataaaactaagggcgtaaccgaaatcggttgaaccgaaaccggttagtataaaagcagacattttatgcaccaaaagagaactgcaatgtttcaggacccacaggagcgacccggaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcaaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacaatatatgccaaacaccacttacaaatattttaaatgtactaaaaactagtaatgcaaaggcagcaatgttagcaaaatttaaagagttatacggggtgagttttacagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactatatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacgctggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaactattaggcagcacttggccaaccactccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatacacaacattactggcgtgctttttgctttgcttttgtgtgcttttgtgtgtctgcctattaatacgtccgctgcttttgtctgtgtctacatacacatcattaatactattggtattactattgtggataacagcagcctctgcgtttaggtgttttattgtatatattgtatttgtttatataccattatttttaatacatacacatgcacgctttttaattacataatgtatatgtacataatgtaattgttacatataattgttgtataccataacttactattttttcttttttatttttatatataatttttttttggtttgtttgtttgttttttaataaactgttctcacttaacaatgcgacacaaacgttctgcaaaacgcacaaaacgtgcatcggctacccaactttataaaacatgcaaacaggcaggtacatgtccacctgacattatacctaaggttgaaggcaaaactattgctgatcaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccatccccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaagttaaagttgtagaccctgcttttgtaaccactcccactaaacttattacatatgataatcctgcatatgaaggtatagatgtggataatacattatattttcctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttgagtactattgatcctgcagaagaaatagaattacaaactataacaccttctacatatactaccacttcacatgcagcctcacctacttctattaataatggcttatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaaccccccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtttatatgtttgtatgtgcttgtatgtgcttgtaaatattaagttgtatgtgtgtttgtatgtatggtataataaacacgtgtgtatgtgtttttaaatgcttgtgtaactattgtgtgatgcaacataaataaacttattgtttcaacacctactaattgtgttgtggttattcattgtatataaactatatttgctacaatctgtttttgttttatatatactatattttgtagcgccagcggccattttgtagcttcaaccgaattcggttgcatgctttttggcacaaaatgtgtttttttaaatagttctatgtcagcaactatagtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgttttgggttacacatttacaagcaacttatataataatactaa

SEQ ID NO: 20 - DNA, 33 bp, synthetic construct
  misc_feature 1..33: note = Primer
  source 1..33: mol_type = other DNA
ggatccgccaccatggagagcgacgagagcggc

SEQ ID NO: 21 - DNA, 27 bp, synthetic construct
  misc_feature 1..27: note = Primer
  source 1..27: mol_type = other DNA
gaattcttagcgagatccggtggagcc

SEQ ID NO: 22 - DNA, 41 bp, unidentified
  source 1..41: mol_type = other DNA; note = human immunodeficiency virus
tacgccaaaaattttgactagcggaggctagaaggagagag

SEQ ID NO: 23 - DNA, 233 bp, unidentified
  source 1..233: mol_type = other DNA; note = human immunodeficiency virus
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 24 - DNA, 118 bp, unidentified
  source 1..118: mol_type = other DNA; note = human immunodeficiency virus
ttttaaaagaaaaggggggattggggggtacagtgcaggggaaagaatagtagacataatagcaacagacatacaaactaaagaattacaaaaacaaattacaaaattcaaaatttta

SEQ ID NO: 25 - DNA, 590 bp, Woodchuck hepatitis virus
  source 1..590: mol_type = other DNA
aatcaacctctgattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 26 - DNA, 1239 bp, Homo sapiens
  source 1..1239: mol_type = other DNA
atgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 27 - DNA, 180 bp, unidentified
  source 1..180: mol_type = other DNA; note = human papillomavirus type 16
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 28 - DNA, 250 bp, unidentified
  source 1..250: mol_type = other DNA; note = human papillomavirus type 16
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 29 - DNA, 350 bp, unidentified
  source 1..350: mol_type = other DNA; note = cytomegalovirus
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 30 - DNA, 1162 bp, unidentified
  source 1..1162: mol_type = other DNA; note = human papillomavirus type 16
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 31 - DNA, 1687 bp, unidentified; identical to SEQ ID NO: 16
  source 1..1687: mol_type = other DNA; note = Epstein-Barr Virus

SEQ ID NO: 32 - DNA, 1926 bp, unidentified
  source 1..1926: mol_type = other DNA; note = Epstein-Barr Virus
atgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtga

(19) United States
(12) Patent Application Publication
(10) Pub. No.: US 2025/0179150 A1
(43) Pub. Date: Jun. 5, 2025

(54) NON-INTEGRATING VIRAL DELIVERY SYSTEM AND METHODS RELATED THERETO

(71) Applicant: American Gene Technologies International Inc., Rockville, MD (US)

(72) Inventors: Charles David PAUZA, Rockville, MD (US); Tyler LAHUSEN, Rockville, MD (US)

(21) Appl. No.: 19/037,524

(22) Filed: Jan. 27, 2025

Related U.S. Application Data
(63) Continuation of application No. 16/308,335, filed on Dec. 7, 2018, now abandoned.
Application No. 16/308,335 is a continuation of application No. PCT/US2016/066185, filed on Dec. 12, 2016.
(60) Provisional application No. 62/431,760, filed on Dec. 8, 2016.
(60) Provisional application No. 62/347,552, filed on Jun. 8, 2016.

(51) Int. Cl. A61K 39/00

(52) U.S. Cl. CPC C07K 16/10; A61K 35/76; A61K 38/1866; A61K 48/00; A61K 48/005; A61P 31/18; C07K 14/43504; C07K 14/475; C07K 14/49; C07K 14/51; C07K 16/1045; C07K 16/22; C07K 16/2863; C07K 16/32; C12N 15/113; C12N 15/86; A61K 9/0014; A61K 9/0019; A61K 9/0053; A61K 2039/505; A61K 2039/5256; C07K 2317/14; C12N 2310/141; C12N 2710/20022; C12N 2740/15043; C12N 2740/16043; C12N 2830/60

(57) ABSTRACT

A non-integrating viral delivery system is disclosed. The system includes a viral carrier, wherein the viral carrier contains a defective integrase gene; a heterologous viral episomal origin of replication; a sequence encoding at least one initiator protein specific for the heterologous viral episomal origin of replication, wherein expression of the sequence encoding the at least one initiator protein specific for the heterologous viral episomal origin of DNA replication is inducible; and at least one gene, gene product, shRNA, siRNA, miRNA, or other RNA of interest.

### CROSS-REFERENCE TO RELATED APPLICATIONS

This application is a U.S. national phase filing under 35 U.S.C. § 371 of PCT/US2017/036433 filed on Jun. 7, 2017, entitled “NON-INTEGRATING VIRAL DELIVERY SYSTEM AND METHODS RELATED THERETO,” which claims priority to U.S. Provisional Patent Application No. 62/347,552 filed on Jun. 8, 2016 entitled “NON-INTEGRATING VIRAL DELIVERY SYSTEM AND METHODS OF USE THEREOF,” U.S. Provisional Patent Application No. 62/431,760 filed on Dec. 8, 2016 entitled “NON-INTEGRATING VIRAL DELIVERY SYSTEM AND METHODS RELATED THERETO,” and PCT/US2016/066185 filed on Dec. 12, 2016 entitled “NON-INTEGRATING VIRAL DELIVERY SYSTEM AND METHODS RELATED THERETO,” the disclosures of which are incorporated herein by reference.

### SEQUENCE LISTING

The Sequence Listing that was submitted in this application is incorporated herein by reference. The text file of the Sequence Listing is named 7061201600_SL.txt and the file size is 48,800 bytes. The text file was submitted electronically via EFS-Web on Dec. 7, 2018.

### FIELD OF INVENTION

The present invention relates generally to the field of viral vectors and systems for the delivery of genes and other therapeutic, diagnostic, or research uses. More specifically, embodiments of the present invention relate to non-integrating viral vectors and systems for the delivery of genes and other therapeutic, diagnostic, or research uses.

### BACKGROUND TO THE INVENTION

Viral vectors have been used to transduce genes and other therapeutic nucleic acid constructs into target cells owing to their specific virus envelope-host cell receptor interactions and viral mechanisms for gene expression. As a result, viral vectors have been used as vehicles for the transfer of genes into many different cell types including, but not limited to, isolated tissue samples, tissue targets in situ and cultured cell lines. The ability to both introduce and express foreign genes in a cell is useful for the study of gene expression and the elucidation of cell lineages and pathways as well as providing the potential for therapeutic interventions such as gene therapy and various types of immunotherapy.

Several viral systems including lentivirus murine retrovirus, adenovirus, and adeno-associated virus have been proposed as potential therapeutic gene transfer vectors. However, many hurdles have prevented robust utilization of these as approved therapeutics. Research and development hurdles include, but are not limited to, stability and control of expression, genome packaging capacity, and construct-dependent vector stability. In addition, in vivo application of viral vectors can be limited by host immune responses against viral structural proteins and/or transduced gene products, which can result in deleterious anti-vector immunological effects.

Researchers have attempted to find stable expression systems as a way of overcoming some of these hurdles. One approach utilizes recombinant polypeptides or gene regulatory molecules, including small RNA, in such expression systems. These systems employ chromosomal integration of a transduced retrovirus genome, or at least a portion thereof, into the genome of the host cell. An important limitation with these approaches is that the sites of gene integration are generally random, and the number and ratio of genes integrating at any particular site are often unpredictable. Thus, vectors that rely on chromosomal integration result in permanent maintenance of the recombinant gene that may exceed the therapeutic interval, and plasmid or other non-replicating DNA is poorly controlled and may decay before completing a desired therapeutic interval.

Another approach is the use of a transient expression system. Under a transient expression system, the expression of the gene of interest is based on non-integrated plasmids, and hence the expression is typically lost as the cell undergoes subsequent division or the plasmid vectors are destroyed by endogenous nucleases. Accordingly, transient gene expression systems typically do not lead to sufficient expression over time and typically require repeated treatments, which are generally understood to be undesirable features.

### SUMMARY OF THE INVENTION

A stable viral delivery system and methods are provided. In various aspects, the delivery system includes a transient expression system. According to one aspect, the delivery system is non-integrating. In another aspect the delivery system is both non-integrating and transient.

In various aspects and embodiments, the system variously includes one or all of a viral carrier, wherein the viral carrier contains a defective integrase gene; a heterologous viral episomal origin of DNA replication; a sequence encoding at least one initiator protein specific for the heterologous viral episomal origin of DNA replication, wherein expression of the sequence encoding the at least one initiator protein specific for the heterologous viral episomal origin of DNA replication is inducible; and at least one gene, gene product, shRNA, siRNA, miRNA, or other RNA of interest. The viral carrier may be a lentivirus. The heterologous viral episomal origin of DNA replication may be from a papillomavirus. The heterologous viral episomal origin of DNA replication may be from a human papillomavirus or a bovine papillomavirus.

The heterologous viral episomal origin of DNA replication may be from a human papillomavirus type 16 (HPV16). The heterologous viral episomal origin of DNA replication may be from a long control region (LCR) of HPV16. The heterologous viral episomal origin of DNA replication may include SEQ ID NO: 1. Optionally, the heterologous viral episomal origin of DNA replication may include a 5′ truncation of SEQ ID NO: 1. The heterologous viral episomal origin of DNA replication may include a 5′ truncation of at least about 200 nucleotides, or at least about 300 nucleotides, or at least about 400 nucleotides, or at least about 500 nucleotides, or at least about 600 nucleotides, or at least about 700 nucleotides of SEQ ID NO: 1. The heterologous viral episomal origin of DNA replication may include at least about 80% sequence identity, or at least about 85% sequence identity, or at least about 90% sequence identity, or at least about 95% sequence identity, or at least about 98% sequence identity with Frag 1 (SEQ ID NO: 2) (also referred to herein as Fragment 1), or Frag 2 (SEQ ID NO: 3) (also referred to herein as Fragment 2), or Frag 3 (SEQ ID NO: 4) (also referred to herein as Fragment 3), or Frag 4 (SEQ ID NO: 5) (also referred to herein as Fragment 4) of the LCR of HPV16. The heterologous viral episomal origin of DNA replication may include Frag 1 (SEQ ID NO: 2), or Frag 2 (SEQ ID NO: 3), or Frag 3 (SEQ ID NO: 4), or Frag 4 (SEQ ID NO: 5) of the LCR of HPV16.

The at least one initiator protein specific for the heterologous viral episomal origin of DNA replication may include E1 or an operative fragment thereof. The at least one initiator protein specific for the heterologous viral episomal origin of DNA replication may include E2 or an operative fragment thereof. The at least one initiator protein specific for the heterologous viral episomal origin of DNA replication may include EBNA-1 or an operative fragment thereof. Optionally, the system may include at least two initiator proteins specific for the heterologous viral episomal origin of replication. The at least two initiator proteins specific for the heterologous viral episomal origin of DNA replication may include either of E1 or E2, alone or in combination, or operative fragments thereof. The sequence encoding the at least one initiator protein may be present on a single discrete plasmid or a non-integrating viral vector. Optionally, the system may include at least two initiator proteins specific for the heterologous viral episomal origin of DNA replication, wherein the sequence encoding the at least two initiator proteins may be present on a single discrete plasmid or a non-integrating viral vector. Optionally, the system may include at least two initiator proteins specific for the heterologous viral episomal origin of DNA replication, wherein the sequence for a first initiator protein and the sequence for a second initiator protein may be present on discrete plasmids or non-integrating viral vectors.

In respect of the disclosed non-integrating viral delivery system, the at least one gene product may include an antibody, an antibody fragment, or a growth factor. The antibody may include an anti-HER2 antibody or a fragment thereof. The growth factor may include vascular endothelial growth factor (VEGF) or a variant thereof. The miRNA may include a CCR5 miRNA.

In another aspect, a pharmaceutical composition is disclosed. The pharmaceutical compositions include the non-integrating viral delivery system disclosed herein and at least one pharmaceutically acceptable carrier.

In another aspect, a method of expressing at least one gene, gene product, shRNA, siRNA, miRNA, or other RNA of interest in a cell is provided. The method includes contacting a cell with an effective amount of a non-integrating viral delivery system, wherein the system includes a viral carrier, wherein the viral carrier contains one or all of a defective integrase gene; a heterologous viral episomal origin of DNA replication; a sequence encoding at least one initiator protein specific for the heterologous viral episomal origin of DNA replication, wherein expression of the sequence encoding the at least one initiator protein specific for the heterologous viral episomal origin of DNA replication is inducible; and at least one gene, gene product, shRNA, siRNA, miRNA, or other RNA of interest.

In another aspect, a method of expressing at least one gene, gene product, shRNA, siRNA, miRNA, or other RNA of interest in a subject in need thereof is provided. The method includes administering to the subject in need thereof an effective amount of a non-integrating viral delivery system, wherein the system includes a viral carrier, wherein the viral carrier contains one or all of a defective integrase gene; a heterologous viral episomal origin of DNA replication; a sequence encoding at least one initiator protein specific for the heterologous viral episomal origin of DNA replication, wherein expression of the sequence encoding the at least one initiator protein specific for the heterologous viral episomal origin of DNA replication is inducible; and at least one gene, gene product, shRNA, siRNA, miRNA, or other RNA of interest. The sequence encoding the at least one initiator protein may be present on a single discrete plasmid, and the at least one initiator protein may include either of E1 or E2, alone or in combination, or operative fragments thereof. The method may further involve administering to the subject in need thereof a first amount of the single discrete plasmid to initiate a first level of expression of the at least one gene, gene product, shRNA, siRNA, miRNA, or other RNA of interest. The method may further involve administering to the subject in need thereof a second amount of the single discrete plasmid to initiate a second level of expression of the at least one gene, gene product, shRNA, siRNA, miRNA, or other RNA of interest. In situations when the second amount is lower than the first amount, the level of expression of the at least one gene, gene product, shRNA, siRNA, miRNA, or other RNA of interest may be reduced. In situations when the second amount is higher than the first amount, the level of expression of the at least one gene, gene product, shRNA, siRNA, miRNA, or other RNA of interest may be increased.

In another aspect, the non-integrating viral delivery system disclosed herein is optimized to produce a low level of basal expression of the at least one gene, gene product, shRNA, siRNA, miRNA, or other RNA of interest. The heterologous viral episomal origin of DNA replication may include at least about 80% sequence identity, or at least about 85% sequence identity, or at least about 90% sequence identity, or at least about 95% sequence identity, or at least about 98% sequence identity with SEQ ID NO: 1 or Frag 1 (SEQ ID NO: 2) of the LCR of HPV16.

In another aspect, the non-integrating viral delivery system disclosed herein is optimized to produce a low level of basal expression of the at least one gene, gene product, shRNA, siRNA, miRNA, or other RNA of interest, and the heterologous viral episomal origin of DNA replication may include SEQ ID NO: 1 or Frag 1 (SEQ ID NO: 2) of the LCR of HPV16.

In another aspect, the non-integrating viral delivery system disclosed herein is optimized to produce a moderate level of basal expression of the at least one gene, gene product, shRNA, siRNA, miRNA, or other RNA of interest, and the heterologous viral episomal origin of DNA replication may include at least about 80% sequence identity, or at least about 85% sequence identity, or at least about 90% sequence identity, or at least about 95% sequence identity, or at least about 98% sequence identity with Frag 2 (SEQ ID NO: 3), Frag 3 (SEQ ID NO: 4), or Frag 4 (SEQ ID NO: 5) of the LCR of HPV16. The system may be optimized to produce a moderate level of basal expression of the at least one gene, gene product, shRNA, siRNA, miRNA, or other RNA of interest, and the heterologous viral episomal origin of DNA replication may include Frag 2 (SEQ ID NO: 3), Frag 3 (SEQ ID NO: 4), or Frag 4 (SEQ ID NO: 5) of the LCR of HPV16.

In another aspect, a method of selecting an optimized non-integrating viral delivery system is disclosed. The method involves selecting a level of basal expression. Thereafter, when a level X is selected, a corresponding Y is selected, wherein Y corresponds to a heterologous viral episomal origin of DNA replication selected to be incorporated into the non-integrating viral delivery system, whereby when X=low; Y comprises SEQ ID NO: 1 or Frag 1 (SEQ ID NO: 2); and when X=moderate; Y comprises Frag 2 (SEQ ID NO: 3), Frag 3 (SEQ ID NO: 4), or Frag 4 (SEQ ID NO: 5) of the LCR of HPV16.

Further aspects include methods of treating, for example, an infectious disease. Further aspects include methods of preventing an infectious disease. In another aspect, methods of enhancing wound healing are disclosed. In another aspect, methods of treating a bone injury are disclosed. Further aspects include methods of treating a hereditary disease using the systems detailed herein.

The foregoing general description and following detailed description are exemplary and explanatory and are intended to provide further explanation of the invention as claimed. Other objects, advantages, and novel features will be readily apparent to those skilled in the art from the following brief description of the drawings and detailed description of the invention.

### BRIEF DESCRIPTION OF THE DRAWINGS

FIG. 1 depicts an exemplary vector-in-vector (VIV) embodiment of the invention.

FIG. 1(A) depicts a linear version of the vector and FIG. 1(B) depicts a circularized version of the vector.

FIG. 2 depicts an exemplary vector-in-vector (VIV) embodiment (also referred to herein as Vector 1) that contains an E1 initiator protein.

FIG. 3 depicts transduction results in 293 T cells from three (3) separate experiments using Vector 1.

FIG. 4 depicts an exemplary vector-in-vector (VIV) embodiment (also referred to herein as Vector 19) that contains both E1 and E2 initiator proteins.

FIG. 5 depicts exemplary vector-in-vector (VIV) embodiments that express (A) mCherry and (B) VEGF, respectively.

FIG. 6 depicts expression of mCherry-positive cells for variously described constructs when E1 and E2 are provided by plasmids (A) or with lentivirus (B), respectively.

FIG. 7 depicts exemplary vector-in-vector (VIV) embodiments used in conjunction with Examples detailed herein.

FIG. 8 depicts expression levels of VEGF for variously described constructs that contain fragment 1 of the HPV16 long control region (LCR).

FIG. 9 depicts exemplary vector-in-vector (VIV) embodiments used in conjunction with Examples detailed herein.

FIG. 10 depicts an exemplary diagram of an episomal form of a non-integrating lentiviral vector according to embodiments of the present invention.

FIG. 11 depicts the genomic relationship between Frag 1, Frag 2, Frag 3, and Frag 4 of the LCR of HPV16.

FIG. 12 depicts an analysis of episomal copy number of the HPV16 on in an integrase-deficient lentiviral vector as described herein.

FIG. 13 depicts an analysis of (A) mCherry expression from integrase-deficient lentiviral vectors containing the HPV LCR and 3′ fragments; and (B) mCherry expression from integrase-deficient vectors which either express or do not express HPV16 E1-T2A-E2 from a single vector.

FIG. 14 depicts an analysis of mCherry expression from an integrase-deficient lentiviral vector containing the HPV LCR following the addition of E1, E1-C, and E2-11.

FIG. 15 depicts expression of an anti-HER2 antibody using an integrase-deficient lentiviral vector containing the HPV LCR as determined by (A) immunoblot; and (B) IgG concentration.

FIG. 16 depicts expression of an anti-EGFR antibody using an integrase-deficient lentiviral vector containing a HPV on sequence.

FIG. 17 depicts a knock-down of CCR5 expression using a lentiviral vector that contains full-length LCR of HPV16.

FIG. 18 depicts a knock-down of CCR5 expression using a lentiviral vector that contains Frag 2 of the LCR of HPV16.

FIG. 19 depicts expression of GFP in cells transduced with a D64V integrase-deficient lentiviral vector using an Epstein-Barr Virus (EBV) oriP sequence.

FIG. 20 depicts a schematic demonstrating the basal and E1-E2-induced episomal copy number for Frag 1, Frag 2, Frag 3, Frag 4, and full-length LCR of HPV16,

FIG. 21 depicts an embodiment for selection of LCR fragment selection based on findings of relative structure-function activity as further described in Examples detailed herein.

### DETAILED DESCRIPTION

Disclosed herein is a stable viral delivery system and methods. In various aspects, the delivery system includes a transient expression system. According to one aspect, the delivery system is non-integrating. In another aspect the delivery system is both non-integrating and transient.

In further aspects, non-integrating, episomally replicating viral vectors (e.g., lentiviral vectors) and methods of using the same are provided. Episomally replicating vectors of the present invention can contain viral components from viruses like Papovaviridae (e.g., bovine papillomavirus or BPV) or Herpesviridae (e.g., Epstein Barr Virus or EBV) or Hepadnaviridae (e.g., Hepatitis B Virus or HBV). Episomal replicating vectors derived from these viruses may contain a replication origin and at least one viral trans-acting factor, e.g., an initiator protein, such as E1 for BPV and EBNA-1 for EBV or HBV polymerase or terminus binding protein of Adenovirus. The process of episomal replication typically incorporates both host cell replication machinery and viral trans-acting factors.

By using heterogeneous viral origins of replication, novel vectors can be engineered with an “off” switch for expression of viral proteins required to recognize the origin of replication. Switching off DNA replication will cause therapeutic DNA levels to dramatically drop over time. Without wishing to be bound by any particular theory, it is believed that the non-replicating DNA simply degrades, such as by nuclease activity and as the host cell undergoes natural apoptosis (cell death) events over time. Eventually such non-replicating DNA may be non-detectible and completely or nearly cleared from the patient over time.

The disclosed systems and methods include reducing or preventing toxicity and toxic effects from over-expression or prolonged expression of transduced genes. Eliminating the gene once DNA replication ceases prevents unwanted gene expression or knockdown of host gene expression in the future. Likewise, combining the benefits of episomal replication into a heterogeneous viral system provides for a platform that can safely and efficiently transduce genes of interest into a variety of cell types.

### Papillomavirus

Papillomaviruses replicate primarily as episomes in mammalian cells. Action of the viral E1 protein, which functions as a DNA helicase, on the viral origin of DNA replication (ori) drives the production of hundreds to thousands of DNA copies per cells depending on differentiation status of infected epithelial cells. Attempts have been made to develop papillomavirus-based gene delivery systems using what became known as “shuttle plasmids.” With a bacterial origin of DNA replication to allow production of DNA in E. coli and a papillomavirus on to allow episomal replication in mammalian cells, a number of studies have been performed to demonstrate safety and durability of gene expression. In most cases, the on came from bovine papillomavirus.

Papillomaviruses have evolved to infect epidermal and epithelial cells. As infected cells differentiate from basal to luminal surfaces, papillomaviruses increase DNA replication and copy number becomes very high until a tremendous dose of virus is released at the lumenal surface. This makes papillomaviruses highly contagious as is apparent from human papillomavirus. The surge in copy number is due primarily to host factors. However, this feature of papillomavirus can be exploited to target transient gene therapy to epidermal and epithelial surfaces.

Certain features of papillomavirus are used in accordance with various aspects and embodiments of the present invention for driving expression and replication of an episomal vector, as well as targeting expression of the vector to specific cell types.

### Epstein Barr Virus (EBV)

Epstein-Barr virus (EBV), also known as human herpesvirus 4, is a member of the herpes virus family. It is one of the most common human viruses, and most people become infected with EBV at some point in their lives.

EBV is a double-stranded DNA virus that contains approximately 85 genes; EBV is known to infect B cells and epithelial cells. EBV is capable of both lytic and latent replication, the latter of which results in a circularized version of the EBV genome translocating to the host cell nucleus where it may be replicated by host cell DNA polymerases.

EBV can undergo latent replication via at least three distinct pathways, but each one involves the expression of Epstein-Barr virus nuclear antigen 1 (EBNA-1), a protein that binds the episomal replication origin and mediates partitioning of the episome during division of the host cell. EBNA-1 plays an integral role in EBV gene regulation, replication, and episomal maintenance.

Certain features of EBV are used in accordance with various aspects and embodiments of the present invention.

### Hepatitis B Virus (HBV)

Hepatitis B virus (HBV) is a member of the hepadnavirus family. It is a common human virus associated with progressive liver fibrosis, hepatitis and hepatocellular carcinoma.

HBV is a double stranded DNA virus that replicates through an RNA intermediate and depends on a viral polymerase. Stable maintenance of HBV in liver cells is due to the presence of covalently-closed viral DNA circular forms that are difficult to eradicate.

Thus, certain features of HBV are used in accordance with various aspects and embodiments of the present invention.

### Retrovirus

Retrovirus is a virus family characterized by encoding a reverse transcriptase capable of generating DNA copies from RNA templates and integration of proviruses into the host cell chromosome. Lentivirus is a genus of retroviruses that can deliver a significant amount of viral nucleic acid into a host cell. Lentiviruses are characterized as having a unique ability to infect/transduce non-dividing cells, and following transduction, lentiviruses integrate their nucleic acid into the host cell's chromosomes.

Infectious lentiviruses have three main genes coding for the virulence proteins gag, pol, and env, and two regulatory genes including tat and rev. Depending on the specific serotype and virus, there may be additional accessory genes that code for proteins involved in regulation, synthesis, and/or processing viral nucleic acids and other replicative functions including counteracting innate cellular defenses against lentivirus infection.

Lentiviruses contain long terminal repeat (LTR) regions, which may be approximately 600 nt long. LTRs may be segmented into U3, R, and U5 regions. LTRs can mediate integration of retroviral DNA into the host chromosome via the action of integrase. Alternatively, without functioning integrase, the LTRs may be used to circularize the viral nucleic acid.

Viral proteins involved in early stages of lentivirus replication include reverse transcriptase and integrase. Reverse transcriptase is a virally encoded, RNA-dependent DNA polymerase. The enzyme uses a viral RNA genome as a template for the synthesis of a complementary DNA copy. Reverse transcriptase also has RNaseH activity for the destruction of the RNA-template that is necessary to DNA second strand synthesis to complete production of the double-stranded DNA ready for integration. Integrase binds both the viral cDNA generated by reverse transcriptase and the host DNA. Integrase processes the LTR before inserting the viral genome into the host DNA. Tat acts as a trans-activator during transcription to enhance the initiation and elongation of RNA copies made from viral DNA. The rev responsive element acts post-transcriptionally, regulating mRNA splicing and transport to the cytoplasm.

Certain features of retroviruses, including lentiviruses, are used in accordance with various aspects and embodiments of the present invention.

### Vector-In-Vector System

A novel vector-in-vector (VIV) system is provided that can precisely regulate the delivery and expression of genes by combining desirable features from various viral species. Many viral vectors, including lentivirus (LV) platforms, may be used. Lentiviral transduction, like most other forms of stable transduction, results in chromosomal integration of the LV payload (e.g., gene of interest). In accordance with various aspects, chromosomal integration is abolished through selective mutations that inactivate the viral integrase gene.

The papillomavirus on plus E1 protein, or the EBV on plus EBNA-1 or the Hepadnavirus termini plus viral polymerase are used herein, as part of the genetic cargo of a heterologous virus that would not ordinarily be able to be maintained episomally. Incorporating this heterogeneous viral replication machinery into a lentiviral vector leaves approximately 5 kb of additional cargo space available to accommodate therapeutic genes of interest.

Additionally, other control elements can be incorporated into the disclosed VIV system. As a non-limiting example, the expression of E1 or E2 or EBNA-1 or HBV polymerase can be driven by an inducible promoter. Further, as a non-limiting example, E1 and/or E2 can be expressed using plasmids or non-integrating viral vectors. Numerous types of inducible promoters are known in the art, and for the purposes of this invention, inducible promoters can include but are not limited to promoters that respond to antibiotics (i.e., tetracyclines, aminoglycosides, penicillins, cephalosporins, polymyxins, etc.) or other drugs, copper and other metals, alcohol, steroids, light, oxygen, heat, cold, or other physical or chemical stimulation. For example, a method of using the disclosed viral system includes employing a tetracycline-inducible gene expression that depends upon a constant supply of the drug for expression of the cargo genes. A compound used to induce the inducible promoter may be added once or repeatedly depending on the duration of episomal replication and timing of cargo delivery that is desired. DNA replication and maintenance of the episome depends variously on E1, E2 and/or EBNA-1 induction, which in turn depends upon an inducer of gene expression (i.e., tetracycline).

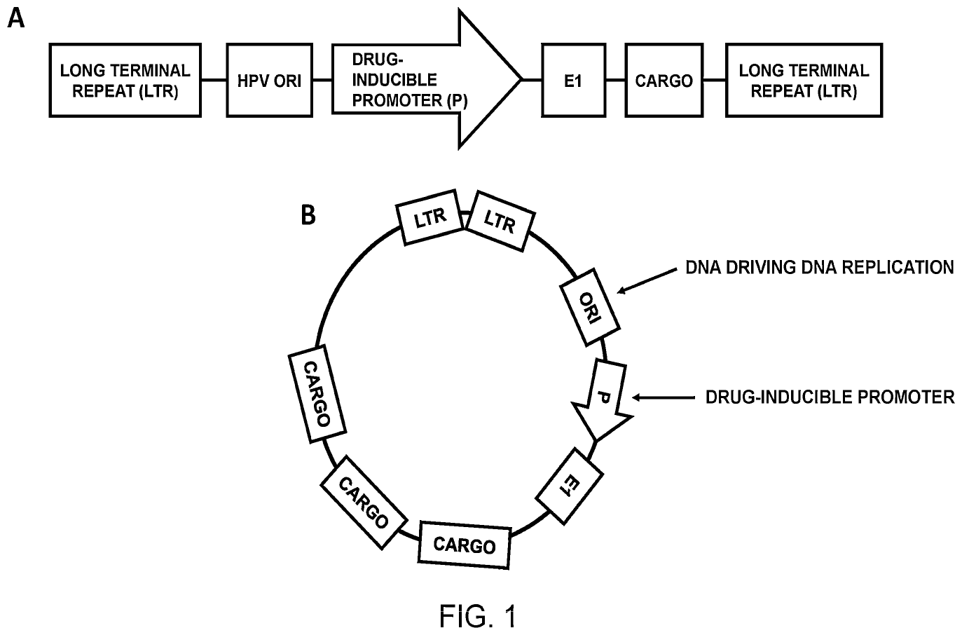

An exemplary VIV system is shown in FIG. 1. The disclosed VIV comprises at least one gene or nucleotide sequence of interest (e.g. cargo as shown in FIG. 1A). The genes or sequences incorporated into the VIV will depend upon the purpose of the VIV. Referring generally to FIG. 1, a lentivirus is packaged with an integrase-defective system or transduction is performed in the presence of clinical drugs used to block integrase activity (e.g., Dolutegravir or Raltegravir). Failing to integrate, the linear double strand vector DNA will generally circularize (e.g. FIG. 1B) using host enzymatic machinery. Optionally, a drug inducible promoter can be activated to express E1 and/or E2 protein if desired, which will in turn drive DNA replication. Therapeutic cargo will be expressed from the integrated cassette(s). In various embodiments, the compound that induces the inducible promoter (also referred to herein as an “inducer”) is withdrawn or terminated. Termination of the inducer will down regulate E1 and/or E2 synthesis. In further embodiments, E1 and/or E2 production is effectively terminated. In either event, this will lead to declining levels of episomal DNA and eventually elimination of the vector construct.

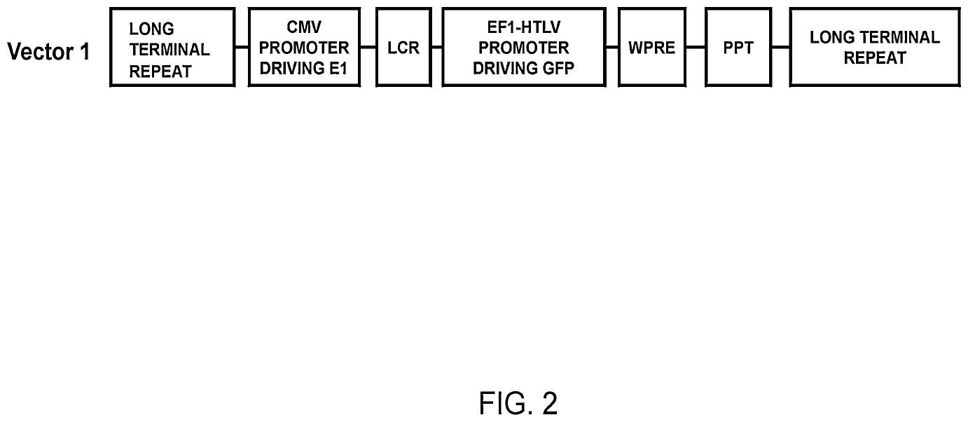

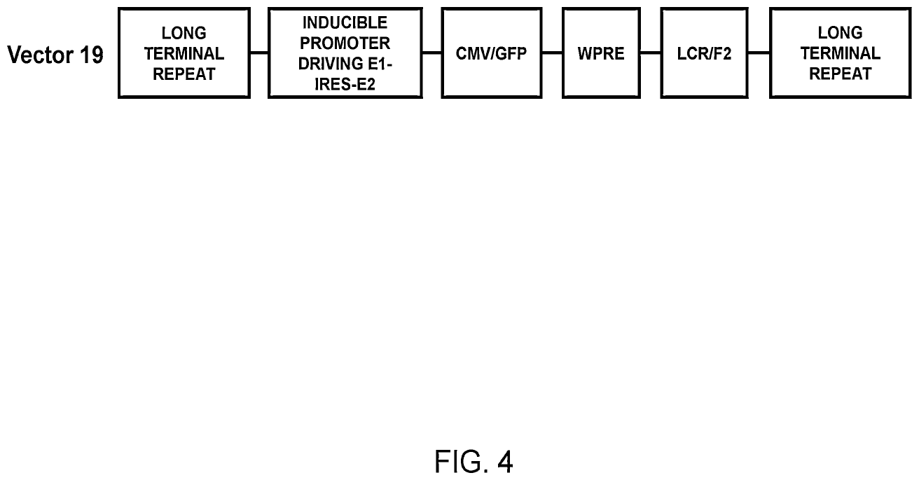

A further exemplary diagram of a VIV system is shown in FIG. 2. An E1 initiator protein is present and the cargo is GFP under an EF1-HTLV promoter. While FIGS. 1 and 2 depict a VIV system containing E1, FIG. 4, as described herein, depicts a VIV system containing both E1 and E2 on a single viral vector. In a further embodiment, in order to express both E1 and E2 from the same mRNA, an internal ribosome entry site (IRES) is added to allow for re-initiation of protein translation. Initiator proteins such as E1 and E2 can also be expressed on separate plasmids or non-integrating lentiviral vectors.

A further exemplary diagram of a VIV system is shown in FIG. 4. The genetic cargo is represented by a CMV/GFP cassette. The cargo gene sequences may be amplified by polymerase chain reaction (PCR). For example, synthetic oligonucleotide primers can be used, such as primers which are identical to the 5′ end of the cargo gene and/or complementary to the 3′ end of the cargo gene. The 5′ primer can be extended from its 5′ end with a recognition site for an endonuclease. The 3′ primer can also be extended at its 3′ end with the complement for an endonuclease recognition. The resulting amplified cargo gene sequences can be annealed into a suitable vector, such as a lentiviral vector. Non-limiting examples of the genetic cargo include CMV/VEGF, CMV/anti-epidermal growth factor receptor (EGFR), an anti-HER2 antibody, or a miRNA suppressing C—C chemokine receptor type 5 (CCR5).

Suitable expression of the cargo may be determined by an appropriate assay. For example, DNA copy numbers can be measured by quantitative PCR. Protein products translated from non-limiting examples such as Vector 1 or Vector 19 (as described herein) can be measured, for example, by analytical flow cytometry. An ELISA assay may be used to detect the presence of certain cargo, such as a secreted protein, such as VEGF. A Western blot technique may also be used to detect certain cargo such as an antibody, such as anti-EGFR. Further, monitoring a reduction in cell surface expression of a cargo protein, such as a chemokine receptor such as CCR5, can also be employed.

In respect of the cargo, and serving as a non-limiting example, the gene encoding platelet-derived growth factor (PDGF) can be incorporated as a gene along with shRNA, siRNA, miRNA, and/or other gene-silencing RNA of interest into a VIV used to promote wound healing. The disclosed VIV system is not limited to a particular type of gene or sequence that can be expressed.

The disclosed VIV can incorporate numerous therapeutic or prophylactic genes or sequences including, for example, sequences that encode antibodies directed to an antigen associated with an infectious disease or cancer (including antigens on replicating pathogens and antigens that are exogenous toxins and antigens on tumor cells), platelet derived growth factor, vascular endothelial growth factor, brain derived growth factor, nerve growth factor, human growth factor, human chorionic gonadotropin, cystic fibrosis transmembrane conductance regulator (CFTR), dystrophin or dystrophin-associated complex, phenylalanine hydroxylase, lipoprotein lipases, α- and/or β-thalassemias, factor VIII, bone morphogenetic proteins 1-4, cyclooxygenase 2, vascular endothelial growth factor, chemokine receptor CCR5, chemokine receptor CXCR4, chemokine receptor CXCR5, antisense DNA or RNA against autoimmune antigens involved in colitis, inflammatory bowel disease or Crohn's disease, small interfering RNA that are involved in addiction including miRNA regulating neural attenuation to opiates or alcohol, tumor suppressor genes, genes regulating cell survival including pro- or anti-apoptosis genes and pro- or anti-autophagy genes, genes encoding radiation resistance factors, genes encoding light emitting proteins used for tracking tumor cell metastasis or other cell trafficking phenomena, or a variety of other therapeutically useful sequences that may be used to condition the body for maximum effect of radiation, surgical or chemotherapeutics or to protect tissues against radiation, surgical or chemotherapeutics, to modify the host or graft tissues to improve organ transplantation or to suppress hyprerreactivity especially in the airway.

Without limiting any of the foregoing, cargo can include diagnostic proteins such as GRP and mCherry, as well as cDNAs, micoRNAs, shRNAs, and antibodies. Further, cargo can include specific cargo such as VEGF and BMP, as described herein.

In further aspects, it is desirable to maintain the genes in episomal form in a VIV system as a “safety switch.” For example, where a particular gene product is toxic, withdrawal of the inducer molecule will reduce or terminate DNA replication. Episome numbers will subsequently decline, and the gene and vector will eventually disappear. Unlike traditional regulated gene expression as the safety switch, the disclosed expression construct is degraded by endogenous nucleases and diluted by cell division until it has effectively disappeared, thereby preventing any short- or long-term breakthrough expression.

In accordance with a further aspect, maintaining a gene, gene product, shRNA, siRNA, miRNA, and/or other gene-silencing RNA of interest in episomal form also allows for regulating the copy number over a broad range and at much higher levels than is achieved by traditional lentivirus transduction.

The disclosed VIV system presents numerous benefits. For instance, episomal DNA is less susceptible to chromosomal modification, which can lead to gene silencing of traditional transduction vectors. Likewise, VIV episomal DNA vectors support active gene delivery at least over short- to medium-range time intervals of about 1 to about 4 months, and possibly longer. In other embodiments of the invention, episomal DNA vectors support active gene delivery over a period of about 1, about 2, about 3, about 4, about 5, about 6, about 7, about 8, about 9, about 10, about 11, or about 12 weeks or longer. In other embodiments, episomal DNA vectors support active gene delivery over a period of about 1 month, 2 months, 3 months, 4 months, 5 months, 6 months, 7 months, 8 months, 9 months, 10 months, 11 months, 12 months, or longer. Any combination of these time periods can also be used in the methods of the invention, e.g., 1 month and 1 week, or 3 months and 2 weeks.

While there are benefits specifically associated with the use of a lentiviral carrier for incorporation of the disclosed VIV system, the disclosed system is not limited to a single type of viral vector. Any DNA virus or virus that uses a DNA intermediate can be used as a carrier for incorporating the VIV system herein, including but not limited to lentivirus, adeno-associated virus (AAV), adenovirus, vaccinia, herpes virus, measles virus, hepadnavirus, parvovirus and murine viruses.

Without limiting any of the foregoing, in an aspect of the invention, a non-integrating viral delivery system is disclosed. The system includes a viral carrier, wherein the viral carrier contains a one or more of a defective integrase gene; a heterologous viral episomal origin of replication; a sequence encoding at least one initiator protein specific for the heterologous viral episomal origin of replication, wherein expression of the sequence encoding the at least one initiator protein specific for the heterologous viral episomal origin of DNA replication is inducible; and at least one gene, gene product, shRNA, siRNA, miRNA, or other RNA of interest. The viral carrier may be a lentivirus. The heterologous viral episomal origin of DNA replication may be from a papillomavirus. The heterologous viral episomal origin of DNA replication may be from a human papillomavirus or a bovine papillomavirus.

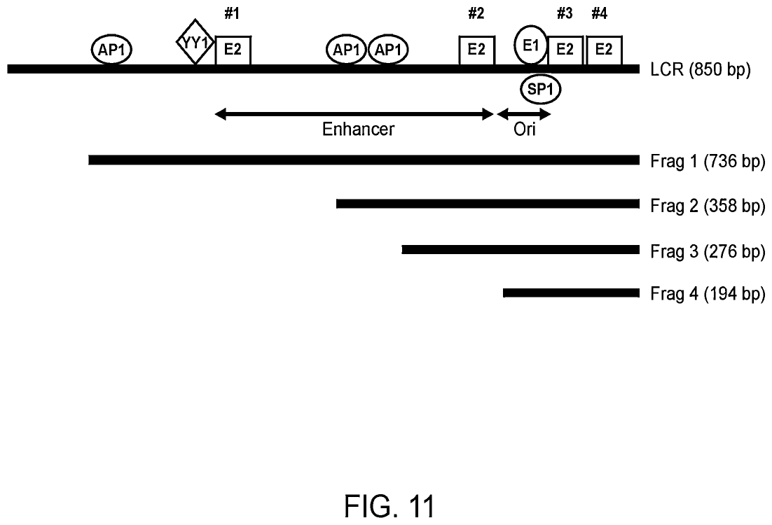

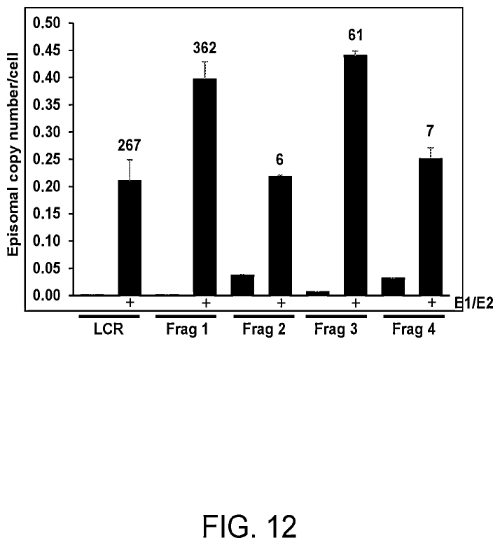

The heterologous viral episomal origin of DNA replication may be from a human papillomavirus type 16 (HPV16). The heterologous viral episomal origin of DNA replication may be from a long control region (LCR) of HPV16. The heterologous viral episomal origin of DNA replication may include SEQ ID NO: 1. Optionally, the heterologous viral episomal origin of DNA replication may include a 5′ truncation of SEQ ID NO: 1. The heterologous viral episomal origin of DNA replication may include a 5′ truncation of at least about 200 nucleotides, or at least about 300 nucleotides, or at least about 400 nucleotides, or at least about 500 nucleotides, or at least about 600 nucleotides, or at least about 700 nucleotides of SEQ ID NO: 1. The heterologous viral episomal origin of DNA replication may include at least about 80% sequence identity, or at least about 85% sequence identity, or at least about 90% sequence identity, or at least about 95% sequence identity, or at least about 98% sequence identity with Frag 1 (SEQ ID NO: 2), or Frag 2 (SEQ ID NO: 3), or Frag 3 (SEQ ID NO: 4), or Frag 4 (SEQ ID NO: 5) of the LCR of HPV16. The heterologous viral episomal origin of DNA replication may include Frag 1 (SEQ ID NO: 2), or Frag 2 (SEQ ID NO: 3), or Frag 3 (SEQ ID NO: 4), or Frag 4 (SEQ ID NO: 5) of the LCR of HPV16. Without limiting any of the foregoing or the Examples detailed herein, the genomic organization of the LCR is depicted in FIG. 11. In addition to the fragments detailed herein, additional fragments can be created by deletion 5′ and 3′ regions of the LCR. Further, mutations, substitutions, additions and/or deletions can be made to the full-length LCR or associated fragments. Further, and without limiting the foregoing or the Examples detailed herein, it is understood and within the scope of the embodiments of this invention that components of the vectors detailed herein can be used interchangeably to develop new and/or modified vectors.

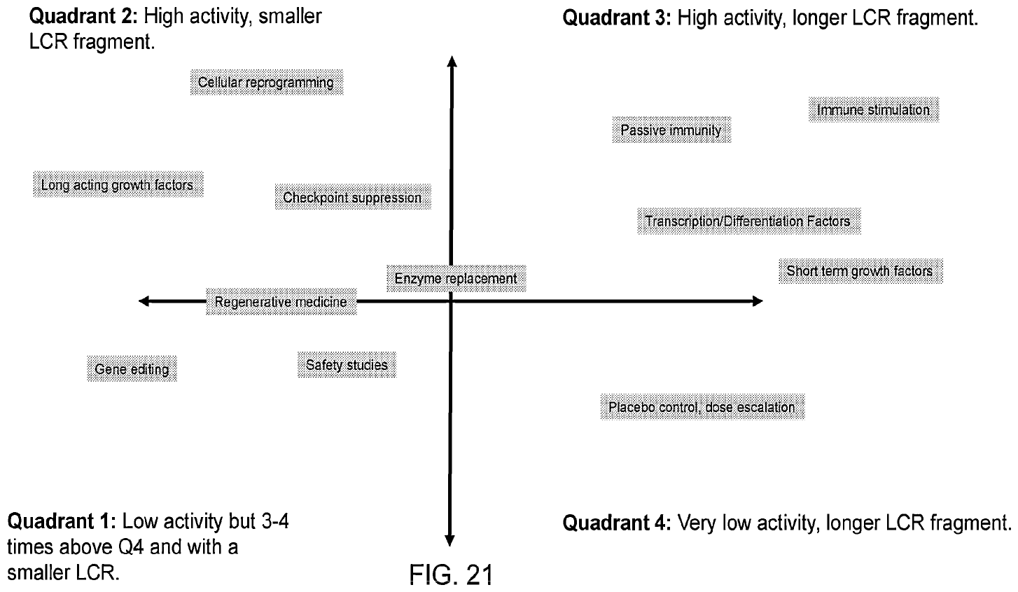

The LCR fragments detailed herein can be selected and used depending on a disease target. As shown in FIG. 21 and in Table 1, depending on the disease target of interest, a LCR fragment correlating to a particular Quadrant (see: FIG. 21) can be selected.

TABLE 1
Summary of Disease Targets and Related Description

| Disease Target or | |
|---|---|
| Biological Property | Example |
| Regenerative Medicine | Therapy for chronic degenerative neurological or systemic |
| | diseases. |
| Cellular reprogramming | Cause changes in the phenotype, function, behavior, growth |
| | rate or life-span via transgene expression of biological |
| | molecules capable of increasing, decreasing or preventing gene |
| | expression at the level of transcription, processing, translation |
| | or posttranslational modification. |
| Long acting growth factors | Protein or peptide factors that are required to sustain tissue |
| | function or integrity. |
| Checkpoint suppression | Modulating the immune checkpoint system to increase |
| | immunity or decrease tumor growth or the number of infected |
| | cells. |
| Enzyme replacement | Short-term delivery of a required growth factor or other |
| | protein that might be needed to bridge an existing regimen or |
| | test the suitability of replacing protein injection with DNA |
| | delivery. |
| Immune stimulation | Protein, peptide, regulatory RNA or other cellular |
| | modifications required to activate and/or direct an immune |
| | response. This may include expression of cytokines or |
| | expression of adhesion, receptor, co-signaling or co- |
| | stimulation proteins on the cell surface, or introduction of |
| | chimeric antigen receptors and/or natural antigen receptors to |
| | direct cellular recognition. |
| Gene editing | CRISPR, zinc finger nuclease, TALEN and other guided DNA |
| | modification systems that are used for gene editing and are not |
| | suitable for permanent or long-term expression in cells. |
| Safety studies | This broad category encompasses situations where the |
| | program objective is to introduce highly durable gene therapy |
| | such as integrating lentivirus vector, but an intermediate step is |
| | needed to assess safety of a proposed DNA construct and/or to |
| | obtain objective clinical responses justifying the introduction |
| | of a longer-lasting version of the same DNA construct. |
| Passive immunity | Transient expression of a protective antibody or antigen |
| | receptor to protect against anticipated pathogen encounters, |
| | bridge existing immunotherapies, combine with chemotherapy |
| | or radiation therapy, and direct immunity to cancer, infectious |
| | disease, or long-term pathology that might include |
| | neurodegenerative disease or activators of autoimmunity |
| | targets. |
| Transcription/Differentiation | Cellular factors including protein, lipid, DNA and/or RNA |
| Factors | may be needed to alter the fate of individual cells including |
| | fetal or adult stem cells where long-term expression would be |
| | detrimental to fully differentiated function or create a risk for |
| | malignant disease. |
| Short term growth factors | Protein, peptide, DNA or RNA molecules intended to |
| | stimulate cellular activity for the purposes of inducing cell |
| | growth, tissue formation, blood vessel formation, muscle |
| | growth, nerve growth, skin growth and other objective clinical |
| | responses where long term factor expression is detrimental to |
| | cell or tissue function. |
| Placebo control, dose | A placebo control with the same cargo but extremely low |
| escalation | expression to monitor the impact of vector delivery on clinical |
| | trial outcomes where vectors include the therapeutic gene |
| | cargo but expression is undetectable due to very low gene |
| | dose. |

Accordingly, based on the intended disease target, a vector system can be designed using Quadrant 1, Quadrant 2, Quadrant 3, or Quadrant 4 components (see: FIG. 21). Selecting Quadrant 1 components provide for transient basal expression of genetic cargo using the described vector systems. In most cases, the DNA copies numbers will be roughly 20-times below the highest levels that can be achieved with this system. Careful selection of promoters driving expression of the DNA cargo will further increase the flexibility and tissue specificity of this system. Selecting Quadrant 2 provides for high episomal DNA copy numbers with potentially very high gene expression levels again depending on promoter selection. Further, the use of shorter LCR fragments increases the size of DNA inserts that can be incorporated as cargo. Selecting Quadrant 3 also provides for high episomal copy numbers but slightly less than can be obtained in Quadrant 2. The advantage of Quadrant 3 is very low basal levels of episomal DNA making the system highly controllable by the introduction or not, of E1/E2 proteins. Selecting Quadrant 4 ensures very low expression such as might be required for a placebo control or initial dose of a dose escalation clinical trial or dosing test to establish optimal levels for a desired indication.

Accordingly, the selection process can be correlated to the LCR fragments employed herein thus rendering the disclosed systems highly adaptable depending on the disease target and the intended biological response.

The at least one initiator protein specific for the heterologous viral episomal origin of DNA replication may include E1 or an operative fragment thereof. The at least one initiator protein specific for the heterologous viral episomal origin of DNA replication may include E2 or an operative fragment thereof. The at least one initiator protein specific for the heterologous viral episomal origin of DNA replication may include EBNA-1 or an operative fragment thereof. Optionally, the system may include at least two initiator proteins specific for the heterologous viral episomal origin of replication. The at least two initiator proteins specific for the heterologous viral episomal origin of DNA replication may be E1 and E2 or operative fragments thereof. The sequence encoding the at least one initiator protein may be present on a single discrete plasmid. Optionally, the system may include at least two initiator proteins specific for the heterologous viral episomal origin of replication, wherein the sequence encoding the at least two initiator proteins may be present on a single discrete plasmid. Optionally, the system may include at least two initiator proteins specific for the heterologous viral episomal origin of replication, wherein the sequence for a first initiator protein and the sequence for a second initiator protein may be present on discrete plasmids.

In respect of the disclosed non-integrating viral delivery system, the at least one gene product may include an antibody, an antibody fragment, or a growth factor. The antibody may include an anti-HER2 antibody or a fragment thereof. The growth factor may include vascular endothelial growth factor (VEGF) or a variant thereof. The miRNA may include a CCR5 miRNA.

### Methods

Aspects of the invention include methods of administering a VIV system to a patient in need thereof, wherein the VIV system encodes at least one, at least two, at least three, at least four, or at least five genes of interest. Given the versatility and therapeutic potential and the disclosed VIV system, a VIV system according to aspects of the invention may encode genes or nucleic acid sequences that include but are not limited to an antibody directed to an antigen associated with an infectious disease or a toxin produced by the infectious pathogen, platelet derived growth factor, vascular endothelial growth factor, brain derived growth factor, nerve growth factor, human growth factor, human chorionic gonadotropin, cystic fibrosis transmembrane conductance regulator (CFTR), dystrophin or dystrophin-associated complex, lipoprotein lipases, α- and/or β-thalassemias, factor VIII, bone morphogenetic proteins 1-4, cyclooxygenase 2, vascular endothelial growth factor, chemokine receptor CCR5, chemokine receptor CXCR4, chemokine receptor CXCR5, antisense DNA or RNA against autoimmune antigens involved in colitis, inflammatory bowel disease or Crohn's disease, small interfering RNA that are involved in addiction including miRNAs regulating neural attenuation to opiates or alcohol, tumor suppressor genes, genes regulating cell survival including pro- or anti-apoptosis genes and pro- or anti-autophagy genes, genes encoding radiation resistance factors, genes encoding light emitting proteins used for tracking tumor cell metastasis or other cell trafficking phenomena, or a variety of other therapeutically useful sequences that may be used to condition the body for maximum effect of radiation, surgical or chemotherapeutics or to protect tissues against radiation, surgical or chemotherapeutics, to modify the host or graft tissues to improve organ transplantation or to suppress hyperreactivity especially in the airway.

Further, and without limiting any of the foregoing, in another aspect, a method of expressing at least one gene, gene product, shRNA, siRNA, miRNA, or other RNA of interest in a cell is provided. The method includes contacting the cell with an effective amount of a non-integrating viral delivery system, wherein the system includes a viral carrier, wherein the viral carrier contains a defective integrase gene; a heterologous viral episomal origin of replication; a sequence encoding at least one initiator protein specific for the heterologous viral episomal origin of replication, wherein expression of the sequence encoding the at least one initiator protein specific for the heterologous viral episomal origin of DNA replication is inducible; and at least one gene, gene product, shRNA, siRNA, miRNA, or other RNA of interest.

In another aspect, a method of expressing at least one gene, gene product, shRNA, siRNA, miRNA, or other RNA of interest in a subject in need thereof is provided. The method includes administering to the subject in need thereof an effective amount of a non-integrating viral delivery system, wherein the system includes a viral carrier, wherein the viral carrier contains a defective integrase gene; a heterologous viral episomal origin of replication; a sequence encoding at least one initiator protein specific for the heterologous viral episomal origin of replication, wherein expression of the sequence encoding the at least one initiator protein specific for the heterologous viral episomal origin of DNA replication is inducible; and at least one gene, gene product, shRNA, siRNA, miRNA, or other RNA of interest. The sequence encoding the at least one initiator protein may be present on a single discrete plasmid, and the at least one initiator protein may be either of E1 or E2, alone or in combination, or fragments thereof. The method optionally includes administering to the subject in need thereof a first amount of the single discrete plasmid to initiate a first level of expression of the at least one gene, gene product, shRNA, siRNA, miRNA, or other RNA of interest. The method optionally includes administering to the subject in need thereof a second amount of the single discrete plasmid to initiate a second level of expression of the at least one gene, gene product, shRNA, siRNA, miRNA, or other RNA of interest. In situations when the second amount is lower than the first amount, the level of expression of the at least one gene, gene product, shRNA, siRNA, miRNA, or other RNA of interest may be reduced. In situations when the second amount is higher than the first amount, the level of expression of the at least one gene, gene product, shRNA, siRNA, miRNA, or other RNA of interest may be increased.

### Infectious Disease

Methods of treating or preventing infectious disease are provided. Prophylactic delivery of monoclonal antibodies to high risk individuals is presently practiced, such as individuals at high risk of contracting an infectious disease, due to health status or geographic location. The prophylactic delivery includes delivery protective antibodies against a lethal viral agent, such as to protect individuals moving through an endemic region (e.g., military and aid workers entering an Ebola-infected region). Vaccines are largely untested for diseases such as Ebola or Lassa Fever virus or Dengue fever or Chikungunya virus or Plasmodium spp. causing malaria, and chronic expression of prophylactic antibody genes through the use of integrating vectors carries unknown health risks. Thus, there is a significant medical need for effective antibody expression that must be high but transient.

The disclosed VIV system and methods of delivering high copy numbers of a gene, gene product, shRNA, siRNA, miRNA, and/or other gene-silencing RNA of interest for a limited period satisfy this medical need. A non-limiting example of a gene product that can be delivered for treating an infectious disease is an antibody specific for the infectious disease in question.

In one aspect, the invention is directed to methods of treating, preventing, or minimizing conditions, symptoms, or side effects associated with infectious disease. In certain embodiments, the infectious disease can be human immunodeficiency virus (HIV), human T cell leukemia virus, Ebola virus, Lassa fever virus, dengue fever, Zika virus, malaria, tuberculosis, rabies, vaccinia virus or other infectious diseases. In some embodiments, a VIV system can be administered prophylactically or following infection with an infectious disease.

In another aspect, a VIV system can be used to prevent an infectious disease. Subjects suspected of having an increased risk of contracting a particular infection disease can receive administrations of a prophylactically effective amount of a VIV encoding an antibody that specifically targets the infectious disease in question.

In certain embodiments, the infectious disease can be human immunodeficiency virus (HIV), human T cell leukemia virus, Ebola virus, Lassa fever virus, dengue fever, Zika virus, malaria, tuberculosis, rabies, vaccinia virus or other infectious diseases. In certain embodiments, a VIV vector can be administered prophylactically or following infection with an infectious disease.

### Wound Healing

In another embodiment, the invention is directed to methods of treating, preventing, or minimizing conditions, symptoms, or side effects associated with wound healing. The disclosed composition can be administered systemically or directly to a wound after an accident, injury, or surgery. In the case of surgery, a VIV system may be administered prophylactically in order to expedite healing. In the case of a wound from an accident, injury, or surgery, a VIV system may be administered sometime after the formation of the wound. For instance, the VIV system may be administered within about 1, about 2, about 3, about 4, about 5, about 10, about 12, about 24, about 36, about 48, about 60, about 72, about 84, about 96, about 108, about 120, or about 168 hours of the formation of a wound.

Another application of the methods and compositions of the invention is transient delivery of VIV constructs capable of expressing platelet growth factor that would accelerate wound healing. A high dose of platelet-derived growth factor (PDGF), related growth factors, fragments thereof, and nucleotide mutants related thereto is required very quickly but transiently. The disclosed system and methods are ideal for this type of application.

Additional short-term applications include expression of brain-derived growth factor for intermittent treatment of alcohol abuse, nerve growth factor for spinal cord regeneration, and topical applications for skin conditions.

### Bone Disease or Injury

In one embodiment, the disclosed invention is directed to a method of enhancing bone healing, comprising identifying a subject with a bone injury and administering to the subject a therapeutically effective amount of a viral delivery system according to the invention. The viral delivery system comprises a viral carrier, a heterologous viral episomal origin of replication, a sequence encoding an initiator protein specific for the heterologous viral episomal origin of replication, and at least one gene, gene product, shRNA, siRNA, miRNA, and/or other gene-silencing RNA of interest, wherein the viral carrier has a defective integrase gene, and wherein expression of the sequence encoding the initiator protein specific for the heterologous viral episomal origin of DNA replication is under the control of an inducible promoter. The bone injury can be, for example, resulting from an accident, injury, or surgery and may be bone nonunion, or acute fracture or required spinal fusion. In some embodiments, the gene, gene product, shRNA, siRNA, miRNA, and/or other gene-silencing RNA of interest encodes bone morphogenetic proteins 1-4 or cyclooxygenase-2 or vascular endothelial growth factor, or fragments thereof. Further, in certain embodiments mutants of the foregoing are preferable and are within the scope of the invention for treating a bone injury or related disease.

In one embodiment, the disclosed invention is directed to a method of enhancing bone healing, comprising identifying a subject with a bone disease and administering to the subject a therapeutically effective amount of a viral delivery system according to the invention. The viral delivery system comprises a viral carrier, a heterologous viral episomal origin of replication, a sequence encoding an initiator protein specific for the heterologous viral episomal origin of replication, and at least one gene, gene product, shRNA, siRNA, miRNA, and/or other gene-silencing RNA of interest, wherein the viral carrier has a defective integrase gene, and wherein expression of the sequence encoding the initiator protein specific for the heterologous viral episomal origin of DNA replication is under the control of an inducible promoter. The bone disease can be, for example, resulting from an accident, injury, or surgery and may be bone nonunion, or acute fracture or required spinal fusion. Additionally, the bone disease may be from low bone density, low blood flow to the bone, aging, hereditary conditions, and the like. In some embodiments, the gene, gene product, shRNA, siRNA, miRNA, and/or other gene-silencing RNA of interest encodes bone morphogenetic proteins 1-4 or cyclooxygenase-2 or vascular endothelial growth factor.

### Hereditary Genetic Disease

TABLE 2
Summary of Hereditary Genetic Diseases and Implicated
Genetic Factors

| Disorder | Mutation | Chromosome |
|---|---|---|
| 22q11.2 deletion syndrome | D | 22q |
| Alpha-1-anti-trypsin disorder | P | 14q32 |
| 14q32 Angelman syndrome | | |
| Canavan disease | | 17p |
| Charcot-Marie-Tooth disease | | |
| Color blindness | P | X |
| Cri du chat | D | 5 |
| Cystic fibrosis | P | 7q |
| Down syndrome | C | 21 |
| Duchenne muscular dystrophy | D | Xp |
| Haemochromatosis | P | 6 |
| Haemophilia | P | X |
| Klinefelter syndrome | C | X |
| Neurofibromatosis | | 17q/22q/? |
| Phenylketonuria | P | 12q |
| Polycystic kidney disease | P | 16 (PKD1) or 4 |
| | | (PKD2) |
| Prader-Willi syndrome | DC | 15 |
| Sickle-cell disease | P | 11p |
| Tay-Sachs disease | P | 15 |
| Turner syndrome | C | X |

In another embodiment, the invention is directed to methods of treating, preventing, or minimizing conditions, symptoms, or side effects associated with a hereditary genetic disease. Several examples of such hereditary genetic diseases are disclosed in Table 2 herein, along with the causal type of mutation and chromosome involved using the nomenclature below:
P—Point mutation, or any insertion/deletion entirely inside one gene
D—Deletion of a gene or genes
C—Whole chromosome extra, missing, or both (see Chromosome abnormality)
T—Trinucleotide repeat disorders: gene is extended in length

Current gene therapy includes efforts to edit genomic DNA through gene deletion, replacement, or re-sequencing. Various gene therapy systems known in the art, including Talen, CRISPR-Cas9, zinc finger endonuclease, TALEN, and others, rely on delivery of genetic material by lentivirus transduction. But, unlike the disclosed invention, these systems may have unexpected consequences if left active in cells for extended periods because active chromosome modification systems may alter unexpected sites, leading to new genetic diseases including cancer. Truly practical systems for modification of host DNA require transient, well-regulated expression through methods such as the method disclosed herein.

Thus, in one embodiment, the disclosed invention is directed to a method of treating a hereditary genetic disease, comprising identifying a subject with a hereditary genetic disease and administering to the subject a therapeutically effective amount of a viral delivery system according to the invention. The viral delivery system comprises a one or more of a viral carrier, a heterologous viral episomal origin of replication, a sequence encoding an initiator protein specific for the heterologous viral episomal origin of replication, and at least one gene, gene product, shRNA, siRNA, miRNA, and/or other gene-silencing RNA of interest, wherein the viral carrier has a defective integrase gene, and wherein expression of the sequence encoding the initiator protein specific for the heterologous viral episomal origin of DNA replication is under the control of an inducible promoter. The hereditary genetic disease can be, for example, the diseases listed in Table 2, and in some embodiments, the gene, gene product, shRNA, siRNA, miRNA, and/or other gene-silencing RNA of interest encodes non-mutated versions of the genes listed in Table 2. Without limiting the foregoing, the specific hereditary genetic disease can be CF and treatment can be pursued by expressing a non-mutated form of CFTR as detailed herein.

In another embodiment, a guide RNA target sequence is incorporated into the disclosed VIV system. Guide RNA are sequences used to target gene editing machinery to specific sites within the host genome that are mutated or otherwise require correction. Inclusion of guide RNA within the cargo of a VIV system allows for a modification of a section of a chromosome that requires correction, and the same modification will occur within VIV to accelerate degradation and/or dilution by the host. In certain embodiments of the invention, the disclosed viral delivery system comprises one or more of a viral carrier, a heterologous viral episomal origin of replication, a sequence encoding an initiator protein specific for the heterologous viral episomal origin of replication, at least one gene, shRNA, siRNA, miRNA, and/or other gene-silencing RNA of interest, and at least one guide RNA, wherein the viral carrier has a defective integrase gene, and wherein expression of the sequence encoding the initiator protein specific for the heterologous viral episomal origin of DNA replication is under the control of an inducible promoter.

### Ex Vivo Modification of Cells or Tissues

In another aspect, the VIV system may be used to modify cells or tissues that are used for disease therapy. Cells may include, without limitation, primary cells such as lymphocytes, stem cells, epithelial cells, neural cells and others. For example, the VIV system may be used to modify lymphocytes that are redirected to specific disease including cancer, infectious disease or autoimmunity, and where long-term presence of genetically modified cells poses a health risk. For example, a VIV system may also be used to program pluripotent stem cells that require high levels of transcript factors for a defined interval and where the long-term presence of an integrated viral vector is undesirable. Suitable epithelial cells include those used for synthetic skin or other applications. These may require the expression of trophic or growth factors during the initial treatment that would be deleterious to function of the normal tissue after treatment and are best delivered by the VIV systems disclosed herein.

### Doses and Dosage Forms

The disclosed VIV systems allow for short, medium, or long-term expression of genes or sequences of interest and episomal maintenance of the disclosed vectors. Accordingly, dosing regimens may vary based upon the condition being treated and the method of administration.

In one embodiment, VIVs may be administered to a subject in need in varying doses. Specifically, a subject may be administered ≥10⁶ infectious doses (where 1 dose is needed on average to transduce 1 target cell). More specifically, a subject may be administered ≥10⁷, ≥10⁸, ≥10⁹, or ≥10¹⁰ infectious doses. Upper limits of VIV dosing will be determined for each disease indication and will depend on toxicity/safety profiles for each individual product or product lot.

Additionally, VIVs may be administered once or twice a day. Alternatively, VIVs may be administered to a subject in need once a week, once every other week, once every three weeks, once a month, every other month, every three months, every six months, every nine months, once a year, every eighteen months, every two years, every 36 months, or every three years or more.

In various aspects and embodiments, VIVs are administered as a pharmaceutical composition. In embodiments, the pharmaceutical composition comprising VIV can be formulated in a wide variety of nasal, pulmonary, oral, topical, or parenteral dosage forms for clinical application. Each of the dosage forms can contain various disintegrating agents, surfactants, fillers, thickeners, binders, diluents such as wetting agents or other pharmaceutically acceptable excipients. The pharmaceutical composition comprising a VIV can also be formulated for injection.

The VIV composition can be administered using any pharmaceutically acceptable method, such as intranasal, buccal, sublingual, oral, rectal, ocular, parenteral (intravenously, intradermally, intramuscularly, subcutaneously, intracistemally, intraperitoneally), pulmonary, intravaginal, locally administered, topically administered, topically administered after scarification, mucosally administered, via an aerosol, or via a buccal or nasal spray formulation.

Further, the VIV composition can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, and a suspension. Further, the composition may be a controlled release formulation, sustained release formulation, immediate release formulation, or any combination thereof. Further, the composition may be a transdermal delivery system.

In another embodiment, the pharmaceutical composition comprising a VIV can be formulated in a solid dosage form for oral administration, and the solid dosage form can be powders, granules, capsules, tablets or pills. In yet another embodiment, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In a further embodiment, the pharmaceutical composition comprising a VIV can be formulated as a sublingual or buccal dosage form. Such dosage forms comprise sublingual tablets or solution compositions that are administered under the tongue and buccal tablets that are placed between the cheek and gum.

In yet a further embodiment, the pharmaceutical composition comprising a VIV can be formulated as a nasal dosage form. Such dosage forms of the present invention comprise solution, suspension, and gel compositions for nasal delivery.

In one embodiment, the pharmaceutical composition can be formulated in a liquid dosage form for oral administration, such as suspensions, emulsions or syrups. In other embodiments, the liquid dosage form can include, in addition to commonly used simple diluents such as water and liquid paraffin, various excipients such as humectants, sweeteners, aromatics or preservatives. In particular embodiments, the composition comprising VIV or a pharmaceutically acceptable salt thereof can be formulated to be suitable for administration to a pediatric patient.

In one embodiment, the pharmaceutical composition can be formulated in a dosage form for parenteral administration, such as sterile aqueous solutions, suspensions, emulsions, non-aqueous solutions or suppositories. In other embodiments, the non-aqueous solutions or suspensions can include propyleneglycol, polyethyleneglycol, vegetable oils such as olive oil or injectable esters such as ethyl oleate. As a base for suppositories, witepsol, macrogol, tween 61, cacao oil, laurin oil or glycerinated gelatin can be used.

The dosage of the pharmaceutical composition can vary depending on the patient's weight, age, gender, administration time and mode, excretion rate, and the severity of disease.

### Definitions

Words that are not specifically defined herein will be understood to have a meaning consistent with that as understood by persons of ordinary skill in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “administration of” or “administering” an active agent means providing an active agent of the invention to the subject in need of treatment in a form that can be introduced into that individual's body in a therapeutically useful form and therapeutically effective amount.

The terms, “expression,” “expressed,” or “encodes” refer to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

The terms “individual,” “host,” “subject,” and “patient” are used interchangeably herein.

The term “therapeutically effective amount” refers to a sufficient quantity of the active agents of the present invention, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

The term “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis.

As used herein, the term “VIV” refers to a vector-in-vector system for expressing at least one gene, gene product, shRNA, siRNA, miRNA, or other RNA of interest. The term “VIV” is used synonymously with viral delivery system, when used herein.

The following examples are given to illustrate aspects of the present invention. It should be understood, however, that the invention is not to be limited to the specific conditions or details described in these examples. All printed publications referenced herein are specifically incorporated by reference.

### EXAMPLES

### Example 1—VIV for Treating an Infectious Disease

This example demonstrates an exemplary VIV construct for treating an infectious disease.

In this example, FIG. 1A represents an exemplary linear VIV construct for treating Ebola virus, an infectious disease. Herein, at least one of the “cargo” portions depicted in FIG. 1A encodes an antibody that specifically targets Ebola virus. The long terminal repeat (LTR) portions of the exemplary VIV construct can be used to circularize the viral nucleic acid, as shown in FIG. 1B.

Subjects suspected of having or diagnosed as having Ebola virus can receive administrations of a therapeutically effective amount of a VIV encoding an antibody that specifically targets Ebola virus, either alone or in combination with one or more additional agents for the treatment or prevention of Ebola. VIV encoding an antibody that specifically targets Ebola virus and/or additional agents are administered orally, intranasally, intrathecally, intraocularly, intradermally, transmucosally, iontophoretically, topically, systemically, intravenously, subcutaneously, intraperitoneally, or intramuscularly according to methods known in the art or as described herein. Subjects are then evaluated daily for the presence and/or severity of signs and symptoms associated with Ebola virus, including, but not limited to, e.g., fever, fatigue, malaise, weakness, reddened eyes, joint and muscle pain, headache, nausea, vomiting, hemorrhage, and death. Treatments are maintained until such a time as one or more signs or symptoms of Ebola virus infection are ameliorated or eliminated.

It is rationally predicted that subjects suspected of having or diagnosed as having been infected with Ebola virus and receiving therapeutically effective amounts of a VIV encoding an antibody that specifically targets Ebola virus, will display reduced severity or elimination of one or more symptoms associated with Ebola virus infection. It is further rationally predicted that administration of a VIV encoding an antibody that specifically targets Ebola virus in combination with one or more additional agents will have synergistic effects.

These results will show that VIV encoding an antibody that specifically targets Ebola virus is useful in the treatment of Ebola virus.

### Example 2—VIV for Preventing an Infectious Disease

This example demonstrates an exemplary VIV construct for preventing an infectious disease.

In this example, FIG. 1A represents an exemplary linear VIV construct for preventing infection with Ebola virus, an infectious disease. Herein, at least one of the “cargo” portions depicted in FIG. 1A encodes an antibody that specifically targets Ebola virus. The long terminal repeat (LTR) portions of the exemplary VIV construct can be used to circularize the viral nucleic acid, as shown in FIG. 1B.

Subjects suspected of having an increased risk of contracting Ebola virus can receive administrations of a prophylactically effective amount of a VIV encoding an antibody that specifically targets Ebola virus, either alone or in combination with one or more additional agents for the treatment or prevention of Ebola prior to entering an area in which risk of contracting Ebola is increased. VIV encoding an antibody that specifically targets Ebola virus and/or additional agents are administered orally, intranasally, intrathecally, intraocularly, intradermally, transmucosally, iontophoretically, topically, systemically, intravenously, subcutaneously, intraperitoneally, or intramuscularly according to methods known in the art or as described herein. Subjects are then evaluated daily for the presence and/or severity of signs and symptoms associated with Ebola virus, including, but not limited to, e.g., fever, fatigue, malaise, weakness, reddened eyes, joint and muscle pain, headache, nausea, vomiting, hemorrhage, and death. Treatments are maintained until such a time as one or more signs or symptoms of Ebola virus infection are prevented.

It is rationally predicted that subjects suspected of having or diagnosed as having been exposed to Ebola virus and receiving prophylactically effective amounts of a VIV encoding an antibody that specifically targets Ebola virus, will have a reduced risk of contracting Ebola. It is further rationally predicted that administration of VIV encoding an antibody that specifically targets Ebola virus in combination with one or more additional agents will have synergistic effects.

These results will show that VIV encoding an antibody that specifically targets Ebola virus is useful in the prevention of Ebola virus.

### Example 3—VIV for Enhancing Wound Healing

This example demonstrates an exemplary VIV construct for enhancing wound healing.

In this example, FIG. 1A represents an exemplary linear VIV construct for enhancing wound healing. Herein, at least one of the “cargo” portions depicted in FIG. 1A encodes platelet-derived growth factor (PDGF) (SEQ ID NO: 17). The long terminal repeat (LTR) portions of the exemplary VIV construct can be used to circularize the viral nucleic acid, as shown in FIG. 1B.

Subjects with a wound (e.g., from accident, injury, or surgery) can receive administrations of a therapeutically effective amount of a VIV encoding platelet-derived growth factor (PDGF), alone or in combination with one or more additional agents for treating or sterilizing a wound. VIV PDGF and/or additional agents are administered orally, intranasally, intrathecally, intraocularly, intradermally, transmucosally, iontophoretically, topically, systemically, intravenously, subcutaneously, intraperitoneally, or intramuscularly according to methods known in the art or as described herein. Subjects are then evaluated daily to determine the status of the wound. Treatments are maintained until such a time as the wound is healed and scarring is minimized.

It is rationally predicted that subjects with a wound and receiving therapeutically effective amounts of a VIV PDGF will display enhanced wound healing. It is further rationally predicted that administration of VIV encoding PDGF in combination with one or more additional agents will have synergistic effects.

These results will show that VIV encoding PDGF is useful for enhancing wound healing.

### Example 4—VIV for Treating Bone Injury

This example demonstrates an exemplary VIV construct for treating a bone injury.

In this example, FIG. 1A represents an exemplary linear VIV construct for treating a bone injury. Herein, at least one of the “cargo” portions shown in FIG. 1A encodes bone morphogenetic protein (BMP) (SEQ ID NO: 18). The long terminal repeat (LTR) portions of the exemplary VIV construct can be used to circularize the viral nucleic acid, as shown in FIG. 1B.

Subjects suspected of having or diagnosed as having a bone injury can receive administrations of a therapeutically effective amount of a VIV encoding bone morphogenetic protein (BMP), alone or in combination with one or more additional agents for the treatment of the bone injury. VIV encoding BMP and/or additional agents are administered orally, intranasally, intrathecally, intraocularly, intradermally, transmucosally, iontophoretically, topically, systemically, intravenously, subcutaneously, intraperitoneally, or intramuscularly according to methods known in the art or as described herein. Subjects are then evaluated weekly for the presence and/or severity of signs and symptoms associated with the bone injury to determine the rate and strength of healing. Treatments are maintained until such a time as the bone has healed.

It is rationally predicted that subjects suspected of having or diagnosed as having a bone injury and receiving therapeutically effective amounts of a VIV encoding BMP will display reduced severity of injury and enhanced healing. It is further rationally predicted that administration of VIV encoding BMP in combination with one or more additional agents will have synergistic effects.

These results will show that VIV encoding BMP is useful in the treatment of bone injuries or diseases.

### Example 5—VIV for Treating a Hereditary Disease

This example demonstrates an exemplary VIV construct for treating cystic fibrosis (CF).

In this example, FIG. 1A represents an exemplary linear VIV construct for treating CF, a hereditary disease. Herein, at least one of the “cargo” portions depicted in FIG. 1A encodes cystic fibrosis transmembrane conductance regulator (CFTR) (NM_000492). The long terminal repeat (LTR) portions of the exemplary VIV construct can be used to circularize the viral nucleic acid, as shown in FIG. 1B.

Subjects suspected of having or diagnosed as having (CF) can receive administrations of a therapeutically effective amount of a VIV encoding cystic fibrosis transmembrane conductance regulator (CFTR), alone or in combination with one or more additional agents for the treatment of CF. VIV encoding CFTR and/or additional agents are administered orally, intranasally, intrathecally, intraocularly, intradermally, transmucosally, iontophoretically, topically, systemically, intravenously, subcutaneously, intraperitoneally, or intramuscularly according to methods known in the art or as described herein. Subjects are then evaluated weekly for the presence and/or severity of signs and symptoms associated with CF, including, but not limited to, e.g., poor growth, persistent cough, thick sputum and mucus, wheezing, breathlessness, decreased ability to exercise, repeated lung infections, inflamed nasal passage, greasy stools, intestinal blockage, and poor weight gain. Treatments are maintained until such a time as one or more signs or symptoms of CF are ameliorated or eliminated.

It is rationally predicted that subjects suspected of having or diagnosed as having CF and receiving therapeutically effective amounts of a VIV encoding CFTR will display reduced severity or elimination of one or more symptoms associated with CF. It is further rationally predicted that administration of VIV encoding CFTR in combination with one or more additional agents will have synergistic effects.

These results will show that VIV encoding CFTR is useful in the treatment of CF.

### Example 6—VIV Containing E1 to Express Cargo

A vector according to FIG. 2 was produced containing green fluorescent protein gene (GFP) as the cargo. DNA containing the complete Locus Control Region and E1 protein from human papillomavirus type 16 (NCBI accession number U89348; SEQ ID NO: 19) was chemically synthesized. Individual segments and/or coding sequences were initially synthesized. These were amplified by polymerase chain reaction (PCR) using synthetic oligonucleotide primers identical to the 5′ end of green fluorescent protein gene and complementary to the 3′ end of green fluorescent protein. The 5′ primer (SEQ ID NO: 20) was extended from its 5′ end with the recognition site for BamHI or EcoRI endonucleases. The 3′ primer (SEQ ID NO: 21) was extended at its 3′ end with the complement of BamHI, or EcoRI endonuclease recognition sites. The resulting amplified green fluorescent protein gene sequences were then digested with BamHI and EcoRI restriction endonucleases.

A lentiviral vector was obtained from System Biosciences, Inc. The plasmid was cleaved with BamHI and EcoRI enzymes, and mixed with excess amplified green fluorescent protein gene sequences in a 1:3 ratio of insert to vector.

Enzymatic activity was then stopped by heat inactivation at 70 degrees Celsius for 20 minutes. The above mixture was cooled to room temperature to allow annealing.

The annealing reactions were performed with bacteriophage T4 DNA ligase for 30 minutes at room temperature. 2.5 microliters of the resulting ligation mix were added to 25 microliters of STBL3 competent bacterial cells.

Transfection was then carried out by a brief (1 minute) heat-shock at 42 degrees Celsius.

Bacterial cells were streaked onto agar plates containing ampicillin to obtain bacterial cultures. These cultures were expanded in Luria broth.

To check for insertion of amplified green fluorescent protein gene sequences into the lentivirus vector packaging plasmid, DNA was extracted from the above bacterial cultures and purified by standard methods. Purified DNA was digested with the same endonucleases used to make the construct. Fragment lengths were analyzed by agarose gel electrophoresis, and the amplified green fluorescent protein gene sequences were verified by DNA sequencing using specific primers obtained from Eurofins MWG Operon LLC.

Lentivirus vector stocks were produced as follows. At least two lentiviral packaging plasmids plus the cargo plasmid were co-transfected into HEK cells where viral genes and genomic RNA are expressed, assembled into integrase-deficient lentivirus particles, and released into the culture medium. Cell-free supernatants were produced and collected during the interval of 3-10 days after transfection. Lentivirus particles were purified by standard procedures including a combination of methods that could include centrifugation, transient flow filtration, size exclusion chromatography, size exclusion filtration or ion exchange chromatography. The concentration and biological activity (transducing units per ml) for each stock were determined.

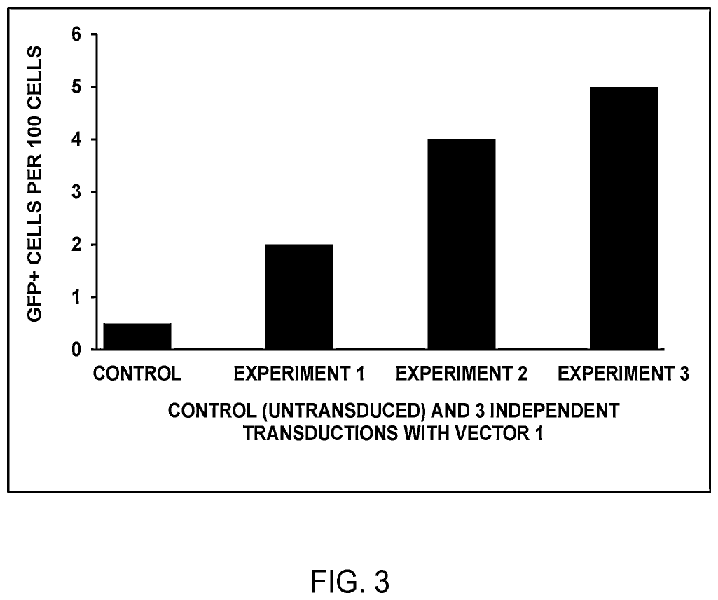

Mammalian cells, including 293T cells, were used to test for lentivirus-derived episome formation, copy number and expression. The 293T cells were transduced with integrase deficient lentivirus particles at a multiplicity of infection ranging from 1 to 10 in the presence of polybrene. Unabsorbed virus was removed by washing cells 3 hours after application, and cells were cultured for 3 days. Cells were observed in a fluorescence microscope and cells expressing GFP were counted. Untransduced 293 T cells were used as a negative control. Data was reported as GFP-positive cells per 100 viable cells in culture. A minimum of 300 cells were counted per microscope field and 5-10 fields were counted for each replicate experiment. Four independent transduction experiments comprising one negative control and three replicate experiments were performed to determine the frequency of transduced cells. The data is depicted in FIG. 3, and shows expression of GFP across three replicate experiments.

### Example 7—VIV Containing E1 and E2 to Express Cargo

Referring to FIG. 4, Vector 19 can be constructed to contain both E1 (SEQ ID NO: 6) and E2 (SEQ ID NO: 7) initiator proteins. Here, the genetic cargo is a CMV/GFP expression cassette under the control of an inducible promoter.

293T cells can be transduced with Vector 19 at a multiplicity of infection ranging from between 1 and 20 transducing units per cell. 3 hours later, cells are washed with medium to remove unadsorbed virions and returned to culture. 12-24 hours after transduction, cells are treated with at least one dosage of a compound that can induce the inducible promoter. Upon addition of a compound that can induce the inducible promoter, E1 and E2 mRNA are transcribed from the episome and combine and assemble on the Locus Control Region Fragment 2 (LCR/F2) (SEQ ID NO: 3) to trigger DNA replication. Lentivirus-derived episomes decay starting approximately 24-36 hours after the cessation of promoter induction. Protein products from the cargo in Vector 19 are measured by analytical flow cytometry.

### Example 8—Introducing E1 and E2 for Expressing Cargo

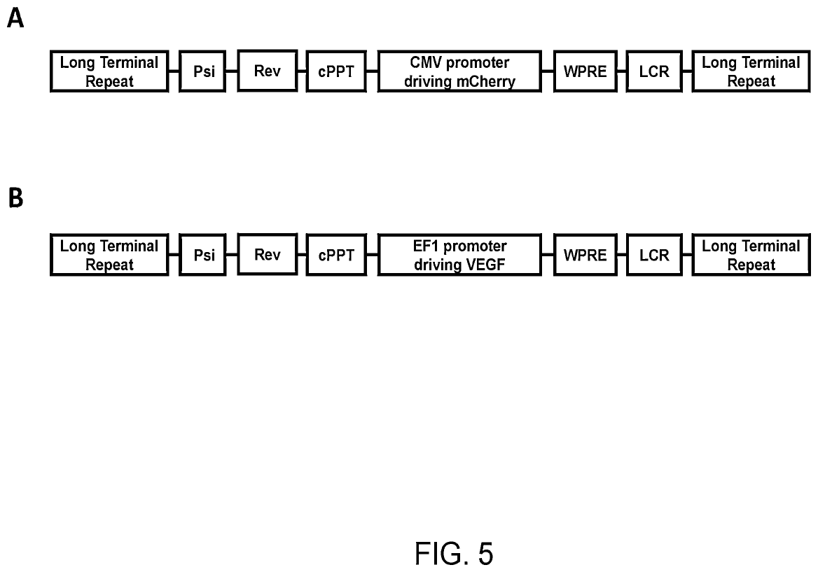

To determine the effect of E1 and E2 in expressing cargo, 293T cells were transduced with a D64V integrase-deficient lentiviral vector (i.e., vector in FIG. 5A) expressing mCherry and the full length HPV16 (SEQ ID NO. 1) long control region (LCR) or 3′ fragments, as described for Fragment 1 (SEQ ID NO: 2), Fragment 2 (SEQ ID NO: 3), Fragment 3 (SEQ ID NO: 4), and Fragment 4 (SEQ ID NO: 5) herein.

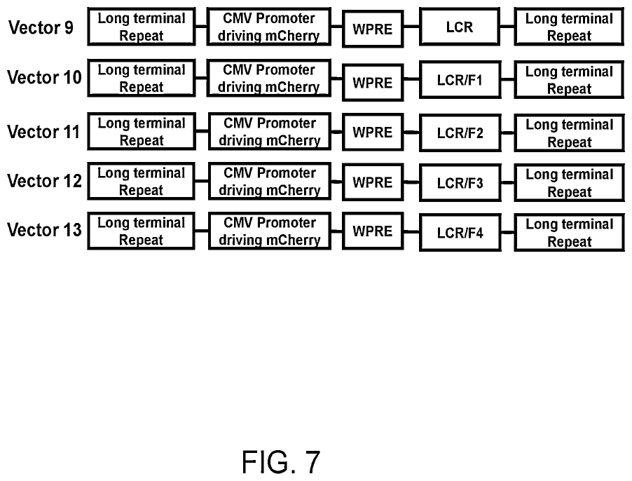

Notably, in reference to FIG. 5A, the full length LCR or the 3′ fragments were utilized in the LCR region depicted in FIG. 5A. More specifically, depictions of the designed constructs are demonstrated as Vectors 9-13 in FIG. 7 herein. Additional elements shown in FIG. 7 refer to: the psi packaging element (SEQ ID NO: 22); the rev element (SEQ ID NO: 23); the cPPT (central polypurine tract) element (SEQ ID NO: 24); and the posttranscriptional regulatory element of woodchuck hepatitis virus (WPRE) (SEQ ID NO: 25).

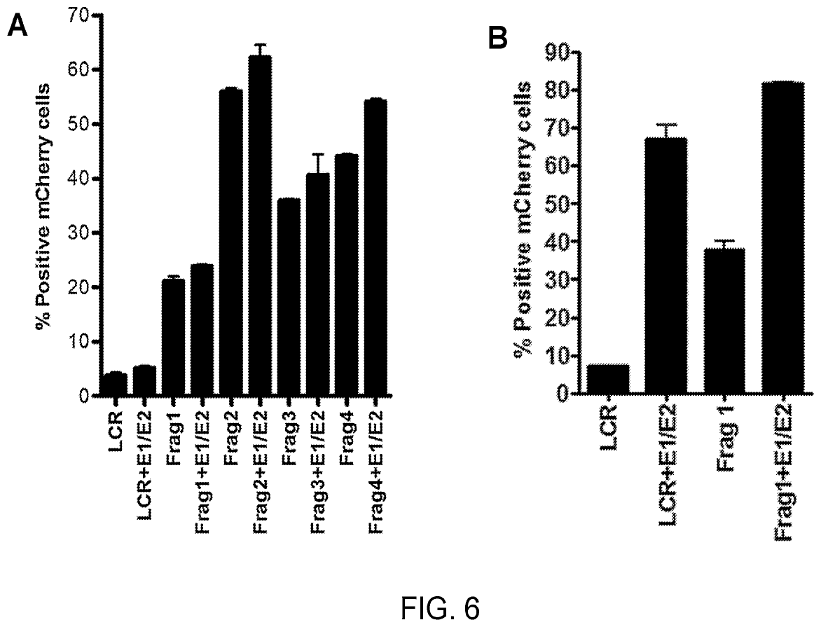

After 24 hours, cells were transfected with plasmids containing HPV16 E1 (SEQ ID NO: 6) and E2 (SEQ ID NO: 7) with Lipofectamine 2000. After 2 days, mCherry expression was analyzed by FACS. The results for these experiments are depicted in FIG. 6A herein.

To contrast with the above experiments wherein E1 and E2 were introduced through plasmids, a second set of experiments were performed as described below. Briefly, 293T cells were transduced with a D64V integrase-deficient lentiviral vector expressing mCherry and the full length HPV16 long control region (LCR) (SEQ ID NO: 1) or a shorter Fragment 1 (SEQ ID NO: 2) based on the generalized vector shown in FIG. 5A herein. At the same time, cells were transduced with lentivirus expressing HPV16 E1 (SEQ ID NO: 6) and E2 (SEQ ID NO: 7). After 2 days, mCherry expression was analyzed by FACS, as shown in FIG. 6B herein. As shown in FIG. 6B herein, a greater percentage of mCherry cells was achieved when E1 and E2 were introduced with the D64V integrase-deficient lentiviral vector expressing mCherry and the full-length LCR (SEQ ID NO: 1) or the shorter fragment 1 (also referred to herein as Fragment 1, and also referred to herein as SEQ ID NO: 2).

The data detailed in this Example demonstrate that when E1 and E2 are expressed via lentiviral-mediated expression, there was stronger expression and thus more activation of HPV on (LCR) full-length and fragments.

Secondly, the data from this Example demonstrates that there is a difference in HPV on activation depending on the size of the LCR region. For example, with reference to FIG. 6, there was a more significant change in the expression of mCherry when using full-length LCR (SEQ ID NO: 1) and Fragment 1 (SEQ ID NO: 2), as compared with Fragments 2 (SEQ ID NO: 3), 3 (SEQ ID NO: 4), and 4 (SEQ ID NO: 5).

### Example 9—Expression of VEGF

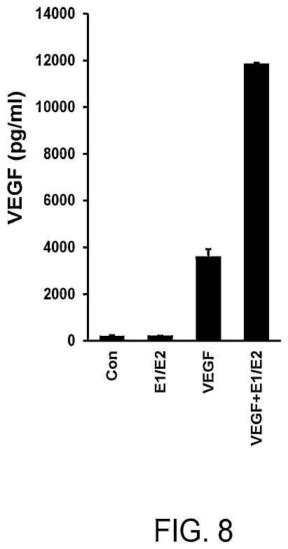

As mentioned herein, VEGF can be selected to be a “cargo” region for treating, among other things, a bone injury. To further analyze the level of VEGF expression, 293T cells were transduced with a D64V integrase-deficient lentiviral vector containing a human cDNA for VEGF (SEQ ID NO: 26) and Fragment 1 (SEQ ID NO: 2) of the HPV16 long control region (LCR) (see: FIG. 5B for generalized description of VEGF-containing vector). At the same time, cells were transduced with lentiviral vectors containing HPV16 E1 (SEQ ID NO: 6) and E2 (SEQ ID NO: 7). After 2 days, cell culture media was collected and analyzed with an ELISA kit for VEGF (Thermo Scientific). As shown in FIG. 8, there was an increase in VEGF levels (3,594 pg/ml) with the VEGF expressing vector, which was further increased with E1 and E2 (11,856 pg/ml).

In a manner similar to the mCherry results from Example 8 above, the results demonstrate that there was a difference in HPV on activation depending on the size of the LCR region. As shown in FIG. 8, there was an approximate 3-fold change in VEGF levels after adding E1/E2. Therefore, the full-length LCR (SEQ ID NO: 1) or Fragment 1 (SEQ ID NO: 2) expresses a gene of interest (i.e., VEGF) at a low level, but when E1/E2 was introduced, there was a strong induction of expression. In contrast, the other fragments tested expressed at a higher initial level and so there was a reduced difference upon introducing E1/E2.

### Example 10—Development of E1 and E2-Containing Vectors

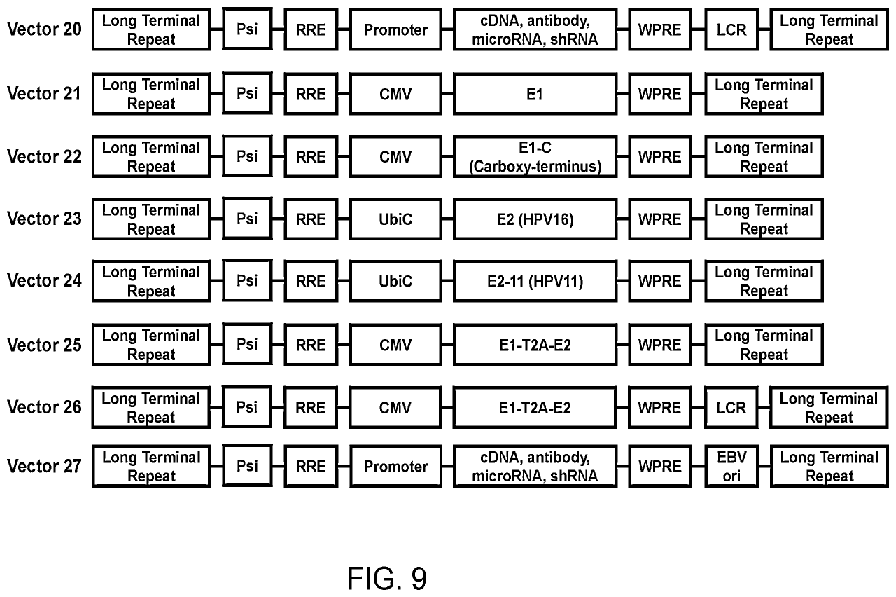

Using standard molecular biology techniques (e.g., Sambrook; Molecular Cloning: A Laboratory Manual, 4^{th} Ed.) as well as the techniques described herein, a series of lentiviral vectors containing the HPV LCRs and E1 and E2 were developed as described in greater detail below. These vectors are also depicted in FIG. 9 herein.

Referring to FIG. 9, Vector 20 was developed and is a general lentiviral vector for expressing a cDNA, a microRNA, or a shRNA. Referring to Vector 20, and from left to right, key components of the vector as developed are: a long terminal repeat portion (SEQ ID NO: 27); a psi packaging element (SEQ ID NO: 22); a rev response element (RRE) (SEQ ID NO: 23); a promoter; a cDNA, microRNA, shRNA or other cargo element; a posttranscriptional regulatory element of woodchuck hepatitis virus (WPRE) (SEQ ID NO: 25), a LCR portion, which can contain fragments of the LCR as detailed herein; and a long terminal repeat portion (SEQ ID NO: 28).

Referring to FIG. 9, Vector 21 was developed and is a lentiviral vector for expressing E1. Referring to Vector 21, and from left to right, key components of the vector as developed are: a long terminal repeat portion (SEQ ID NO: 27); a psi packaging element (SEQ ID NO: 22); a rev response element (RRE) (SEQ ID NO: 23); a CMV promoter (SEQ ID NO: 29); E1 (SEQ ID NO: 6); a posttranscriptional regulatory element of woodchuck hepatitis virus (WPRE) (SEQ ID NO: 25); and a long terminal repeat portion (SEQ ID NO: 28).

Referring to FIG. 9, Vector 22 was developed and is a lentiviral vector for expressing E1-C(carboxy terminus) (SEQ ID NO: 8). Referring to Vector 22, and from left to right, key components of the vector as developed are: a long terminal repeat portion (SEQ ID NO: 27); a psi packaging element (SEQ ID NO: 22); a rev response element (RRE) (SEQ ID NO: 23); a CMV promoter (SEQ ID NO: 29); E1-C(SEQ ID NO: 8); a posttranscriptional regulatory element of woodchuck hepatitis virus (WPRE) (SEQ ID NO: 25); and a long terminal repeat portion (SEQ ID NO: 28).

Referring to FIG. 9, Vector 23 was developed and is a lentiviral vector for expressing E2 (HPV 16) (SEQ ID NO: 7). Referring to Vector 23, and from left to right, key components of the vector as developed are: a long terminal repeat portion (SEQ ID NO: 27); a psi packaging element (SEQ ID NO: 22); a rev response element (RRE) (SEQ ID NO: 23); a UbiC promoter (SEQ ID NO: 30); E2 (HPV16) (SEQ ID NO: 7); a posttranscriptional regulatory element of woodchuck hepatitis virus (WPRE) (SEQ ID NO: 25); and a long terminal repeat portion (SEQ ID NO: 28).

Referring to FIG. 9, Vector 24 was developed and is a lentiviral vector for expressing E2-11 (HPV 11) (SEQ ID NO: 9). Referring to Vector 24, and from left to right, key components of the vector as developed are: a long terminal repeat portion (SEQ ID NO: 27); a psi packaging element (SEQ ID NO: 22); a rev response element (RRE) (SEQ ID NO: 23); a UbiC promoter (SEQ ID NO: 30); E2-11 (HPV11) (SEQ ID NO: 9); a posttranscriptional regulatory element of woodchuck hepatitis virus (WPRE) (SEQ ID NO: 25); and a long terminal repeat element (SEQ ID NO: 28).

Referring to FIG. 9, Vector 25 was developed and is a lentiviral vector for expressing E1-T2A-E2 (SEQ ID NO: 10). Referring to Vector 25, and from left to right, key components of the vector as developed are: a long terminal repeat portion (SEQ ID NO: 27); a psi packaging element (SEQ ID NO: 22); a rev response element (RRE) (SEQ ID NO: 23); a CMV promoter (SEQ ID NO: 29); E1-T2A-E2 (SEQ ID NO: 10); a posttranscriptional regulatory element of woodchuck hepatitis virus (WPRE) (SEQ ID NO: 25); and a long terminal repeat portion (SEQ ID NO: 28).

Referring to FIG. 9, Vector 26 was developed and is a lentiviral vector for expressing E1-T2A-E2 (SEQ ID NO: 10) and full-length LCR (SEQ ID NO: 1) or a fragment thereof (e.g., SEQ ID NOs: 2-5). Referring to Vector 26, and from left to right, key components of the vector as developed are: a long terminal repeat portion (SEQ ID NO: 27); a psi packaging element (SEQ ID NO: 22); a rev response element (RRE) (SEQ ID NO: 23); a CMV promoter (SEQ ID NO: 29); E1-T2A-E2 (SEQ ID NO: 10); a posttranscriptional regulatory element of woodchuck hepatitis virus (WPRE) (SEQ ID NO: 25); a LCR portion, and a long terminal repeat portion (SEQ ID NO: 28).

Still referring to FIG. 9, Vector 27 depicts a general lentiviral vector for expressing, for example, a cDNA, an antibody, a microRNA, or a shRNA. Referring to Vector 27, and from left to right, key components of the vector as developed are: a long terminal repeat portion (SEQ ID NO: 27); a psi packaging element (SEQ ID NO: 22); a rev response element (RRE) (SEQ ID NO: 23); a promoter; a cDNA. microRNA, shRNA or other cargo element; a posttranscriptional regulatory element of woodchuck hepatitis virus (WPRE) (SEQ ID NO: 25); a EBV ori (SEQ ID NO: 31); and a long terminal repeat element (SEQ ID NO: 28).

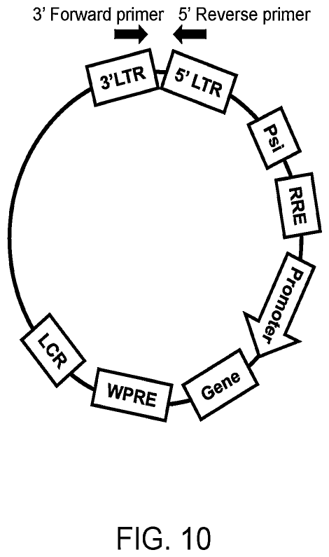

The linear vectors detailed herein circularize intracellularly as shown, for example in FIG. 10, which depicts a circularization of Vector 20 (as shown in FIG. 9). For purposes of experiments detailed herein, FIG. 10 details a primer set as arrows which are located on the 3′ and 5′ Long Terminal Repeats (LTRs). This primer set has been designed to amplify the episomal form of the lentiviral vector, and does not amplify the integrated form of the vector. Appropriate primers for detection of lentiviral episomes contain the following sequences:
3′LTR Fwd CTAATTCACTCCCAACGAAG (SEQ ID NO: 11); and
5′LTR Rev GCCGAGTCCTGCGTCGAGAG (SEQ ID NO: 12).

In the experiments detailed herein, integrase-deficient lentiviral vector copy number was regulated by a combination of utilizing Vector 20 in combination with Vector 21 or Vector 22 or Vector 23 or Vector 24. Alternately, integrase-deficient lentiviral vector copy number was regulated by a combination of utilizing Vector 20 in combination with Vector 25 or Vector 26.

### Example 11—Development of LCR Fragments and Related Vectors

As discussed herein, the LCR portion of the vectors detailed herein can be and were modified through the use of fragments such as Fragment 1 (SEQ ID NO: 2), Fragment 2 (SEQ ID NO: 3), Fragment 3 (SEQ ID NO: 4); and Fragment 4 (SEQ ID NO: 5).

The genomic organization of the LCR and the fragments described herein is depicted in FIG. 11. Therein, the full-length LCR (top portion) contains a series of AP1, YY1, E1, and E2 binding sites. As shown, for example, in FIG. 11, Fragment 1 (SEQ ID NO: 2), Fragment 2 (SEQ ID NO: 3), Fragment 3 (SEQ ID NO: 4), and Fragment 4 (SEQ ID NO: 5) represent increasing LCRs with increasing 5′ truncations which reduces a series of AP1, YY1, and E2 binding sites. Lentiviral vectors which make use of the LCR fragments are detailed herein (e.g., FIG. 7 and related Examples herein).

### Example 12—Testing of Vectors Containing LCR Fragments and E1IE2 Variants

To test vectors containing the various LCR fragments detailed herein, 293T cells were transduced with D64V integrase-deficient lentiviral vectors containing either full length HPV16 long control region (LCR) or Fragment 1 (SEQ ID NO: 2), Fragment 2 (SEQ ID NO: 3), Fragment 3 (SEQ ID NO: 4), and Fragment 4 (SEQ ID NO: 5) as described herein (see, for e.g., FIG. 7 and related Examples herein).

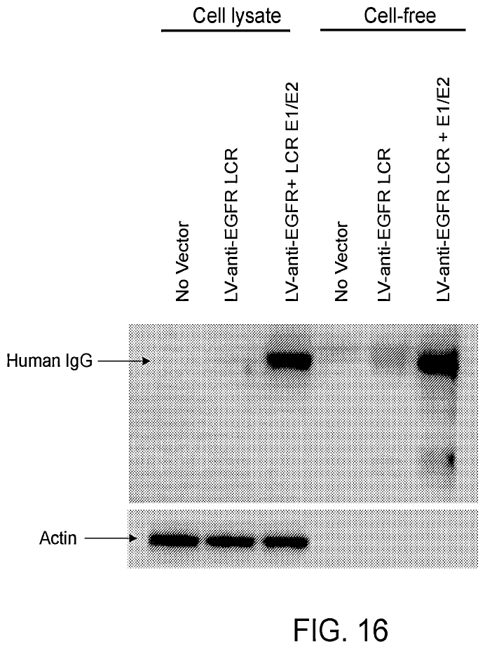

After 24 hours, cells were transfected with plasmids containing HPV16 E1 (SEQ ID NO: 6) and E2 (SEQ ID NO: 7) with Lipofectamine 2000. After 2 days, DNA was extracted for analysis by qPCR. Primers represented by SEQ ID NO: 11 and SEQ ID NO: 12, which are specific for the episomal form of the lentiviral vector were used to determine the episomal copy number. Notably, this primer set amplified only 1- and 2-LTR episomes. The data for this Example is depicted in FIG. 16. Therein, the numbers associated with the LCR and fragments thereof reflect a fold-change increase for each of the conditions following the addition of E1 and E2.

In a separate set of related experiments, analysis was carried out for mCherry expression from integrase-deficient lentiviral vectors containing the HPV LCR and 3′ fragments thereof. Briefly, 293T cells were transduced with D64V integrase-deficient lentiviral vectors expressing mCherry and either full length HPV16 long control region (LCR) or Fragment 1 (SEQ ID NO: 2), Fragment 2 (SEQ ID NO: 3), Fragment 3 (SEQ ID NO: 4), and Fragment 4 (SEQ ID NO: 5). At the same time, cells were transduced with lentivirus expressing HPV16 E1 (SEQ ID NO: 6) and E2 (SEQ ID NO: 7). After 2 days, mCherry expression was analyzed by FACS.

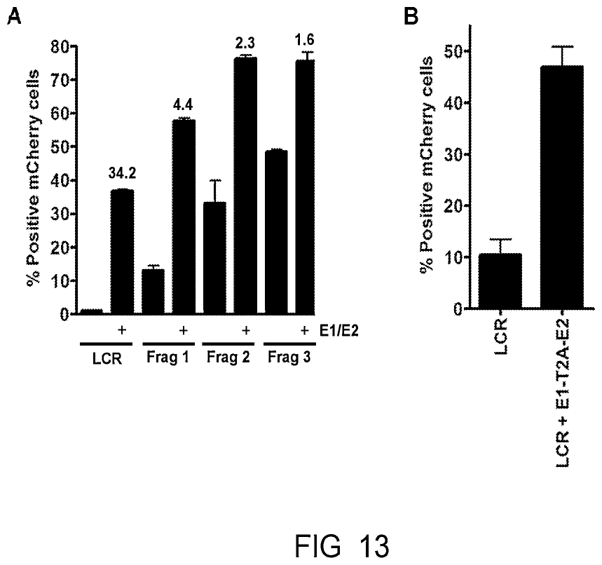

As shown in FIG. 13A, the percent of mCherry cells are identified for each of the tested conditions. The numbers associated with the LCR and fragments thereof reflect a fold-change increase for each of the conditions following the addition of E1 and E2.

In a separate set of related experiments, 293T cells were transduced with a D64V integrase-deficient lentiviral vector expressing mCherry and the full-length HPV16 long control region identified previously as SEQ ID NO: 1. At the same time, cells were transduced with lentivirus expressing HPV16 E1-T2A-E2 (SEQ ID NO: 10) from a single vector (see: Vector 25 from FIG. 9). After 2 days, mCherry expression was analyzed by FACS and the data is depicted in FIG. 13B. As shown therein, transduction with HPV16 E1-T2A-E2 (SEQ ID NO: 10) resulted in a significant increase in positive mCherry cells.

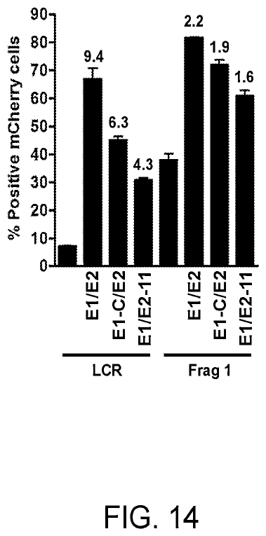

In a separate set of related experiments, an analysis was conducted of mCherry expression using integrase-deficient lentiviral vectors containing HPV LCR following the addition of E1, E1-C, and E2-11. Briefly, 293T cells were transduced with a D64V integrase-deficient lentiviral vector expressing mCherry and HPV16 LCR (SEQ ID NO: 1) or Fragment 1 (SEQ ID NO: 2). At the same time, cells were transduced with HPV16 E1 (i.e., Vector 21 in FIG. 9; and SEQ ID NO: 6) or a E1 carboxy (C)-terminal fragment (i.e., Vector 22 in FIG. 9; and SEQ ID NO: 8) and HPV16 E2 (i.e., Vector 23 in FIG. 9; and SEQ ID NO: 7) or HPV11 E2 (i.e., Vector 24 in FIG. 9; and SEQ ID NO: 9). After 2 days, mCherry expression was analyzed by FACS. As shown in FIG. 14, the percent of mCherry cells are identified for each of the tested conditions. The numbers associated with the tested conditions reflect a fold-change increase for each of the conditions following the addition of E1 and E2.

### Example 13—Antibody Expression

As mentioned herein, one of the features of the disclosed system is the usefulness of the disclosed system to express an antibody. In a series of representative experiments detailed herein, an anti-HER2 antibody was expressed using the lentiviral vector system. Briefly, 293T cells were infected with a D64 integrase-deficient lentiviral vector (i.e., Vector 20) containing an antibody sequence against HER2 (SEQ ID NO: 13) and the HPV LCR (SEQ ID NO: 1) sequence.

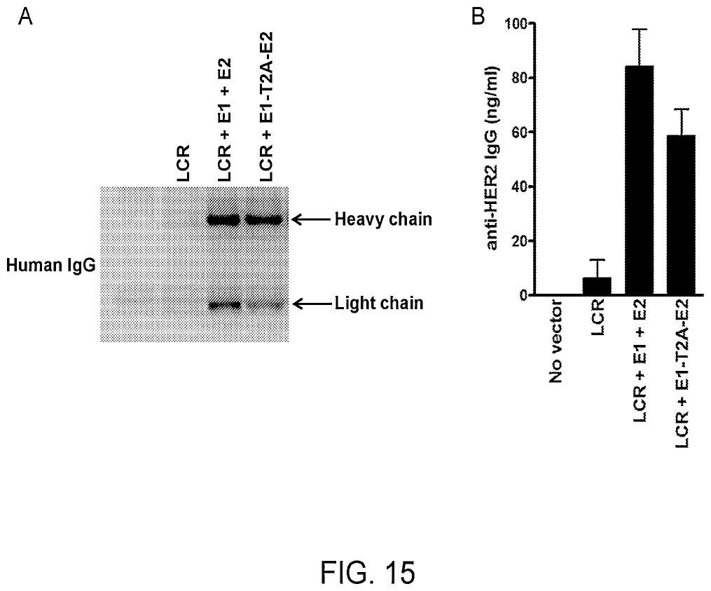

At the same time, cells were infected with lentiviral vectors containing E1 (SEQ ID NO: 6) and E2 (SEQ ID NO: 7). After 3 days, cell culture media was collected. Antibody was purified from the media using Protein A/G agarose beads. An immunoblot was performed using a sheep anti-human antibody (Thermo Scientific). Antibody production was increased with the addition of E1 and E2 as shown in FIG. 15A. Further, as shown in FIG. 15B, anti-HER2 IgG concentration was determined using the EasyTiter IgG kit (Thermo Scientific).

Further, as shown in FIG. 16 herein, additional antibodies can also be expressed using the systems disclosed herein. In FIG. 16, an immunoblot demonstrating expression of an anti-EGFR antibody (SEQ ID NO: 14) is shown. Briefly, 293T cells were infected with a D64 integrase-deficient lentiviral vector containing an antibody sequence against EGFR (see: SEQ ID NO: 14 below) and the HPV fragment 2 (SEQ ID NO: 3).

After 24 hours, cells were infected with lentiviral vectors containing E1 (SEQ ID NO: 6) and E2 (SEQ ID NO: 7). After 3 days, cell lysate and cell culture media was collected. Antibody was purified from media using Protein A/G agarose beads and extracted from cells by cell lysis. An immunoblot was performed using a sheep anti-human antibody (Thermo Scientific) and an anti-actin (Sigma) antibody for a protein loading control for cell lysate. Antibody production was increased in both cell lysate and media with the addition of E1 and E2, as shown in FIG. 16.

### Example 14—microRNA Expression and Knock-Down

As mentioned herein, one of the features of the disclosed system is the usefulness of the disclosed system to express a microRNA. As a non-limiting example, constructs were designed to express microRNA for CCR5 based on the SEQ ID NO: 15.

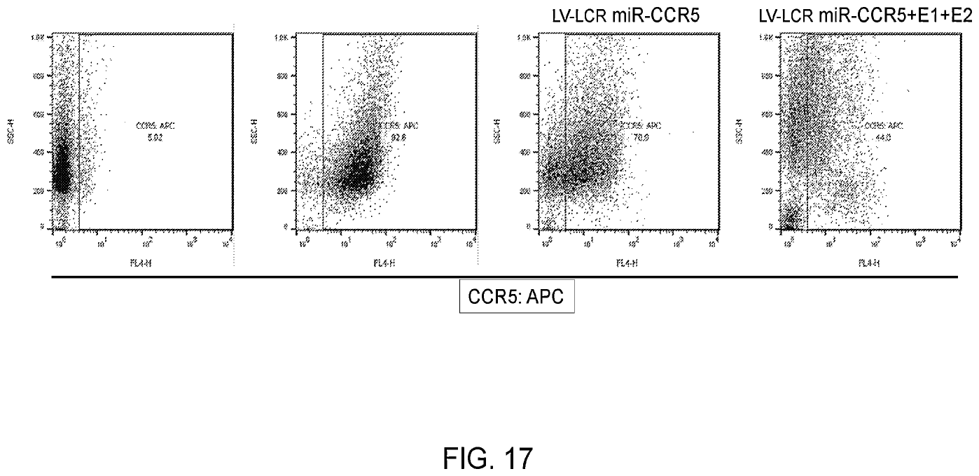

Briefly, HeLa cells expressing CCR5 were infected with a D64 integrase-deficient lentiviral vector (i.e., Vector 20) containing a microRNA sequence against CCR5 (SEQ ID NO: 15) and the full-length HPV LCR (SEQ ID NO: 1) sequence. At the same time, cells were infected with lentiviral vectors containing E1 and E2. After 3 days, cells were collected and analyzed for CCR5 expression by FACS analysis with an anti-CCR5 APC-conjugated antibody. As shown in FIG. 17, the percentage of CCR5 positive cells decreased from 92.6% to 70.9% with LV-LCR miR-CCR5 and to 44% with LV-LCR miR-CCR5 plus E1 and E2.

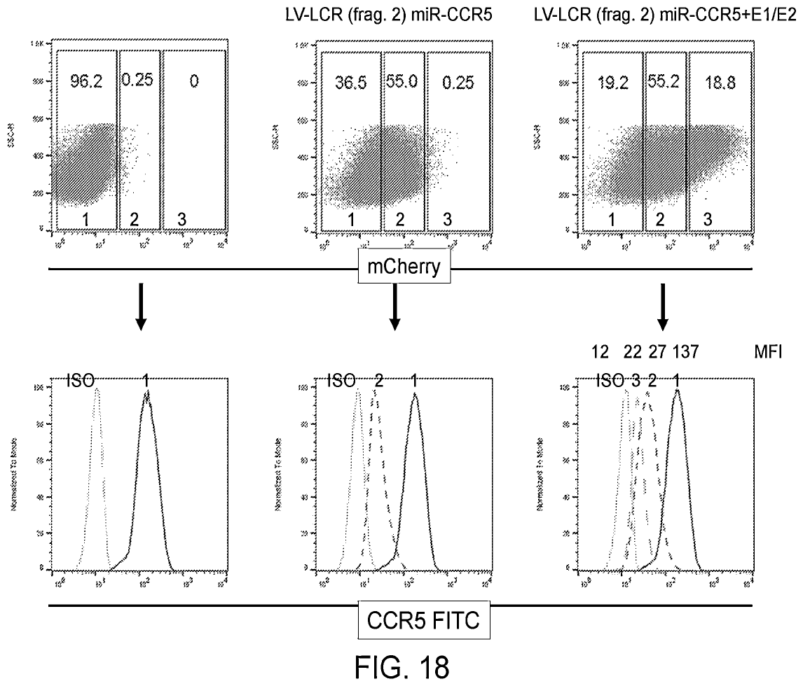

In related experiments, a D64 integrase-deficient lentiviral vector containing a microRNA sequence against CCR5 and the Fragment 2 (SEQ ID NO: 3) LCR sequence were utilized. As shown in FIG. 18, there was a similar decrease in CCR5 expression following the addition of miR-CCR5 and even more so when E1 and E2 were added.

Referring in more detail to FIG. 18, the upper panels show the distribution of cells, each represented by a single dot, based on the level of expression of mCherry. The lower panels show the corresponding change in CCR5 expression that is related to the level of DNA replication and production of a miRNA against CCR5. CCR5 is detected by a fluorescent monoclonal antibody used for staining the cell surface. Without any LV vector (left panels) there is no expression of mCherry (all cells are in Sector 1) and CCR5 expression is uniformly high at around 200 fluorescence intensity units. By adding LV-LCR (Fragment 2; SEQ ID NO: 3) containing miRCCR5 we find cells with basal expression of mCherry (55% of cells now found in the Sector 2) and some reduction in CCR5 expression leading to a new population with fluorescence intensity centered around 30 intensity units (dashed line on the lower, center panel). By adding both LV-LCR miRCCR5 and a non-integrating lentivirus vector expressing E1 and E2 replication proteins, we find 18.8% of cells with highest expression of mCherry (Sector 3) and find a new population (curve 3, gray and dashed line) with even lower CCR5 expression that is less than 20 fluorescence intensity units. These data demonstrate the capacity for a VIV containing LCR Fragment 2 (SEQ ID NO: 3) to express a basal level of miRCCR5 that is biologically active in reducing cell surface expression of the CCR5 protein. Further, the results show the impact of adding E1/E2 DNA replication proteins on vector copy number (related to the expression of mCherry) and increased miRCCR5 expression leading to further reduction in cell surface CCR5 expression.

### Example 15—EBV-Based Initiator Protein

As mentioned herein, initiator proteins such as E1 (SEQ ID NO: 6) and E2 (SEQ ID NO: 7) are used to augment the effectiveness of the systems described herein. An alternate initiator protein that can be used in the current system is EBNA-1 (SEQ ID NO: 32). Accordingly in a series of experiments, 293T cells were transduced with a D64V integrase-deficient lentiviral vector (i.e., Vector 27) expressing GFP and the Epstein-Barr Virus (EBV) OriP sequence (SEQ ID NO: 31).

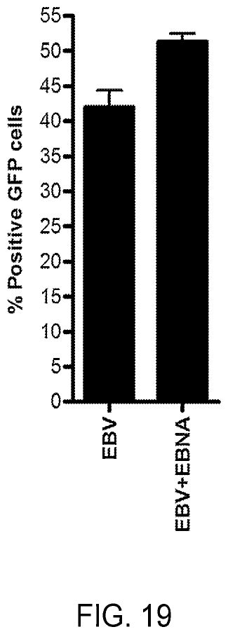

After 24 hours, cells were transfected with a plasmid containing EBV EBNA-1 (SEQ ID NO: 32) with Lipofectamine 2000. After 2 days, GFP expression was analyzed by FACS. As shown in representative data in FIG. 19, EBV plus EBNA resulted in enhanced GFP expression. Accordingly, this data demonstrates that the initiator protein/ori interaction is not limited to E1/E2 interactions but can also include Epstein-Barr viral components.

### Example 16—LCR Fragment Combinations and Analysis

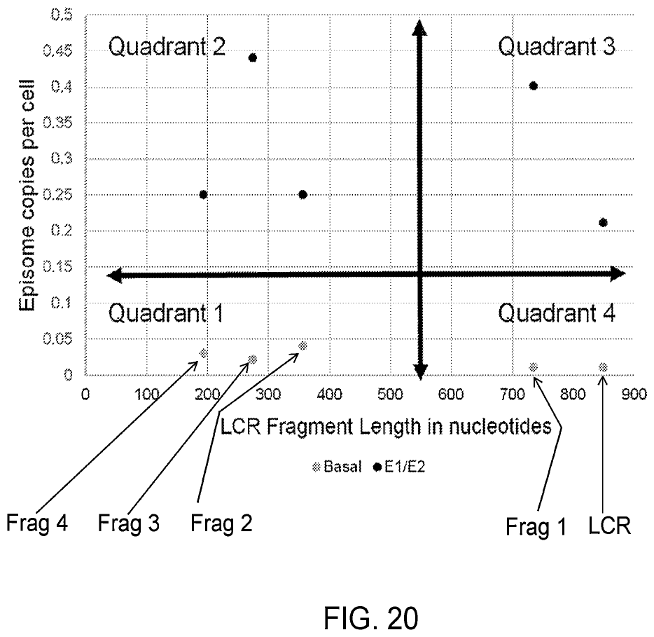

Based on the data detailed herein, varied levels of expression, as determined by episome copies per cell, can be attributed to the various LCR fragments tested herein. As shown in FIG. 20, and moving from right to left, data for full-length LCR (SEQ ID NO: 1), Fragment 1 (SEQ ID NO: 2), Fragment 2 (SEQ ID NO: 3), Fragment 3 (SEQ ID NO: 4) and Fragment 4 (SEQ ID NO: 5) are shown with and without E1/E2.

Referring to both FIGS. 11 and 20, increasing deletions from the 5′ end of LCR remove key functional elements. As detailed herein, basal activity can be defined by the number of episomal DNA copies measured by quantitative PCR assay, when LCR or fragments of LCR are present within a lentivirus-derived episome vector, without added E1/E2 proteins. Inducible activity can be measured by transfecting expression plasmids containing E1 and E2, then introducing a lentivirus-derived episome vector before measuring episomal DNA copy number per cell in a quantitative PCR assay. Similar results were obtained when the E1/E2 protein expression construct was delivered as a non-integrating lentivirus vector. As detailed herein, basal activity has been determined to be highest for Fragments 2, 3 and 4 of the LCR. This indicates that basal activity is suppressed by the presence of a YY1 transcription factor binding site that is present in both LCR and Fragment 1 but not Fragments 2-4, as shown diagrammatically in FIG. 11. Within Fragments 2-4, Fragment 2 had the highest basal expression and was the only fragment to include both AP1 transcription factor binding sites. Thus, basal transcription is increased when the YY1 site is removed and both AP1 sites are preserved. As detailed herein, inducible activity was determined to be highest for Fragments 1 and 3, lower for Fragments 2 and 4, and lowest for intact LCR. There is an unidentified element within LCR that is not present in Fragment 1 and it acts to suppress inducible DNA replication. When the YY1 and AP1 sites are present (Fragment 1), episomal DNA levels are lower compared to removing YY1 and all AP1 sites (Fragment 3). When the AP1 sites are present without YY1 (Fragment 2) or when YY1, AP1 and two of four E2 binding sites are removed (Fragment 4) inducible episomal DNA formation is intermediate and similar to LCR.

As summarized in FIG. 20, the data detailed herein demonstrates definable differences in basal level expression and the ability for expression to be induced. Based on this summary data, at least four quadrants of activity can be defined as shown initially in FIG. 20.

Moving to FIG. 21, the four quadrants represent varying degrees of activity attributable to the LCR and their fragments. For example, as shown in FIG. 21, Quadrant 1 reflects low activity but 3-4 times higher activity than Quadrant 4, and with a smaller LCR fragment. Quadrant 2 reflects high activity, again with a smaller LCR fragment. Quadrant 3 reflects higher activity but this time with a relatively longer LCR fragment. Finally, Quadrant 4 reflects very low activity with a relatively longer LCR fragment.

As detailed in FIG. 21, each Quadrant can reasonably be associated with a particular preferred outcome. As a first example, when a preferred outcome is for use in gene editing, a LCR chosen from Quadrant 1 can be selected. As a second example, when a preferred outcome is cellular reprogramming, a LCR from Quadrant 2 can be selected. As a third example, when a preferred outcome is immune stimulation, a LCR from Quadrant 3 can be selected. As a fourth example, when a preferred outcome is a placebo effect, a LCR from Quadrant 4 can be selected. Importantly, and based on the preferred outcome, varied LCR fragments can be employed using the current system.

### Sequences

The following sequences are referred to herein:

| SEQ ID | | |
|---|---|---|
| NO: | Description | Sequence |
| 1 | HPV16 LCR | AAGGCCAAACCAAAATTTACATTAGGAAAACGAAA |
| | nucleotide | AGCTACACCCACCACCTCATCTACCTCTACAACTGC |
| | sequence (945 | TAAACGCAAAAAACGTAAGCTGTAAGTATTGTATGT |
| | nucleotides; also | ATGTTGAATTAGTGTTGTTTGTTGTGTATATGTTTGT |
| | referred to herein | ATGTGCTTGTATGTGCTTGTAAATATTAAGTTGTAT |
| | as LCR | GTGTGTTTGTATGTATGGTATAATAAACACGTGTGT |
| | | ATGTGTTTTTAAATGCTTGTGTAACTATTGTGTCATG |
| | | CAACATAAATAAACTTATTGTTTCAACACCTACTAA |
| | | TTGTGTTGTGGTTATTCATTGTATATAAACTATATTT |
| | | GCTACATCCTGTTTTTGTTTTATATATACTATATTTT |
| | | GTAGCGCCAGGCCCATTTTGTAGCTTCAACCGAATT |
| | | CGGTTGCATGCTTTTTGGCACAAAATGTGTTTTTTTA |
| | | AATAGTTCTATGTCAGCAACTATGGTTTAAACTTGT |
| | | ACGTTTCCTGCTTGCCATGCGTGCCAAATCCCTGTTT |
| | | TCCTGACCTGCACTGCTTGCCAACCATTCCATTGTTT |
| | | TTTACACTGCACTATGTGCAACTACTGAATCACTAT |
| | | GTACATTGTGTCATATAAAATAAATCACTATGCGCC |
| | | AACGCCTTACATACCGCTGTTAGGCACATATTTTTG |
| | | GCTTGTTTTAACTAACCTAATTGCATATTTGGCATA |
| | | AGGTTTAAACTTCTAAGGCCAACTAAATGTCACCCT |
| | | AGTTCATACATGAACTGTGTAAAGGTTAGTCATACA |
| | | TTGTTCATTTGTAAAACTGCACATGGGTGTGTGCAA |
| | | ACCGATTTTGGGTTACACATTTACAAGCAACTTATA |
| | | TAATAATACTAAACTACAATAATTCATGTATAAAAC |
| | | TAAGGGCGTAACCGAAATCGGTTGAACCGAAACCG |
| | | GTTAGTATAAAAGCAGACATTTTATGCACCAAAAG |
| | | AGAACT |
| 2 | HPV16 LCR | GTGTGTATGTGTTTTTAAATGCTTGTGTAACTATTGT |
| | fragment 1 | GTCATGCAACATAAATAAACTTATTGTTTCAACACC |
| | nucleotide | TACTAATTGTGTTGTGGTTATTCATTGTATATAAACT |
| | sequence (736 | ATATTTGCTACATCCTGTTTTTGTTTTATATATACTA |
| | nucleotides, 209 | TATTTTGTAGCGCCAGGCCCATTTTGTAGCTTCAAC |
| | bases deleted | CGAATTCGGTTGCATGCTTTTTGGCACAAAATGTGT |
| | from 5′ | TTTTTTAAATAGTTCTATGTCAGCAACTATGGTTTAA |
| | terminus; also | ACTTGTACGTTTCCTGCTTGCCATGCGTGCCAAATC |
| | referred to herein | CCTGTTTTCCTGACCTGCACTGCTTGCCAACCATTCC |
| | as Fragment 1) | ATTGTTTTTTACACTGCACTATGTGCAACTACTGAAT |
| | | CACTATGTACATTGTGTCATATAAAATAAATCACTA |
| | | TGCGCCAACGCCTTACATACCGCTGTTAGGCACATA |
| | | TTTTTGGCTTGTTTTAACTAACCTAATTGCATATTTG |
| | | GCATAAGGTTTAAACTTCTAAGGCCAACTAAATGTC |
| | | ACCCTAGTTCATACATGAACTGTGTAAAGGTTAGTC |
| | | ATACATTGTTCATTTGTAAAACTGCACATGGGTGTG |
| | | TGCAAACCGATTTTGGGTTACACATTTACAAGCAAC |
| | | TTATATAATAATACTAAACTACAATAATTCATGTAT |
| | | AAAACTAAGGGC0GTAACCGAAATCGGTTGAACCGA |
| | | AACCGGTTAGTATAAAAGCAGACATTTTATGCACCA |
| | | AAAGAGAACT |
| 3 | HPV16 LCR | TGTGTCATATAAAATAAATCACTATGCGCCAACGCC |
| | fragment 2 | TTACATACCGCTGTTAGGCACATATTTTTGGCTTGTT |
| | nucleotide | TTAACTAACCTAATTGCATATTTGGCATAAGGTTTA |
| | sequence (358 | AACTTCTAAGGCCAACTAAATGTCACCCTAGTTCAT |
| | nucleotides, 587 | ACATGAACTGTGTAAAGGTTAGTCATACATTGTTCA |
| | nucleotides | TTTGTAAAACTGCACATGGGTGTGTGCAAACCGATT |
| | deleted from 5′ | TTGGGTTACACATTTACAAGCAACTTATATAATAAT |
| | terminus; also | ACTAAACTACAATAATTCATGTATAAAACTAAGGGC |
| | referred to herein | GTAACCGAAATCGGTTGAACCGAAACCGGTTAGTA |
| | as Fragment 2) | TAAAAGCAGACATTTTATGCACCAAAAGAGAACT |
| 4 | HPV16 LCR | CTAATTGCATATTTGGCATAAGGTTTAAACTTCTAA |
| | fragment 3 | GGCCAACTAAATGTCACCCTAGTTCATACATGAACT |
| | nucleotide | GTGTAAAGGTTAGTCATACATTGTTCATTTGTAAAA |
| | sequence (276 | CTGCACATGGGTGTGTGCAAACCGATTTTGGGTTAC |
| | nucleotides, 669 | ACATTTACAAGCAACTTATATAATAATACTAAACTA |
| | bases deleted | CAATAATTCATGTATAAAACTAAGGGCGTAACCGA |
| | from 5′ | AATCGGTTGAACCGAAACCGGTTAGTATAAAAGCA |
| | terminus; also | GACATTTTATGCACCAAAAGAGAACT |
| | referred to herein | |
| | as Fragment 3) | |
| 5 | HPV16 LCR | TAGTCATACATTGTTCATTTGTAAAACTGCACATGG |
| | fragment 4 | GTGTGTGCAAACCGATTTTGGGTTACACATTTACAA |
| | nucleotide | GCAACTTATATAATAATACTAAACTACAATAATTCA |
| | sequence (194 | TGTATAAAACTAAGGGCGTAACCGAAATCGGTTGA |
| | nucleotides, 751 | ACCGAAACCGGTTAGTATAAAAGCAGACATTTTATG |
| | nucleotides | CACCAAAAGAGAACT |
| | deleted from 5′ | |
| | terminus; also | |
| | referred to herein | |
| | as Fragment 4) | |
| 6 | E1 HPV16 | ATGGCAGACCCCGCTGGAACAAATGGAGAGGAGGG |
| | codon-optimized | CACTGGGTGTAACGGCTGGTTTTACGTGGAAGCAGT |
| | nucleotide | CGTAGAGAAGAAGACAGGCGACGCCATTTCAGACG |
| | sequence (1,950 | ACGAGAATGAGAACGATAGCGACACTGGTGAGGAT |
| | nucleotides; also | CTTGTGGACTTTATTGTGAACGACAATGACTATCTC |
| | referred to herein | ACCCAGGCAGAAACCGAGACCGCCCACGCCCTCTT |
| | as E1) | CACAGCCCAGGAAGCTAAGCAACATCGGGATGCAG |
| | | TGCAGGTGCTCAAAAGAAAGTACCTGGTTAGTCCTC |
| | | TGTCCGACATCTCTGGATGCGTCGACAATAATATCA |
| | | GTCCAAGGCTGAAGGCTATATGCATAGAGAAGCAG |
| | | TCAAGAGCGGCGAAGAGGAGACTGTTTGAAAGCGA |
| | | GGATAGTGGATACGGGAACACAGAAGTCGAGACCC |
| | | AACAGATGCTCCAGGTGGAGGGTCGCCATGAGACT |
| | | GAGACCCCCTGCTCCCAGTACAGCGGCGGATCAGG |
| | | CGGTGGATGCTCTCAGTACTCCAGTGGGTCCGGCGG |
| | | GGAGGGTGTTTCCGAAAGACACACCATCTGTCAGA |
| | | CCCCCCTGACTAATATTCTGAACGTACTGAAAACAT |
| | | CCAACGCCAAGGCTGCCATGCTGGCGAAGTTTAAG |
| | | GAGCTGTATGGCGTGAGCTTCAGCGAACTGGTGAG |
| | | ACCATTCAAGAGCAACAAGAGCACCTGTTGTGATTG |
| | | GTGTATTGCCGCCTTTGGGCTGACTCCATCCATCGC |
| | | TGACTCTATTAAAACCCTGTTGCAACAGTACTGCCT |
| | | CTACCTGCATATTCAGTCCCTCGCTTGCTCCTGGGG |
| | | AATGGTGGTGCTGCTTCTGGTTCGGTATAAGTGTGG |
| | | CAAAAACAGGGAGACCATCGAGAAGCTCCTTAGTA |
| | | AGCTCCTGTGTGTGTCTCCCATGTGCATGATGATTG |
| | | AACCGCCAAAATTGCGGAGCACGGCCGCCGCCCTG |
| | | TACTGGTACAAAACAGGCATAAGCAACATCAGCGA |
| | | AGTGTATGGTGACACGCCAGAATGGATACAGAGAC |
| | | AGACCGTGCTCCAGCACAGTTTTAACGATTGCACAT |
| | | TTGAGCTGTCTCAGATGGTGCAGTGGGCTTATGATA |
| | | ATGACATTGTAGACGATTCCGAAATAGCGTATAAGT |
| | | ACGCCCAGCTCGCAGATACCAATTCCAATGCCAGCG |
| | | CATTTCTGAAGTCCAATTCACAGGCAAAGATAGTAA |
| | | AGGATTGCGCTACAATGTGCCGCCATTATAAAAGA |
| | | GCGGAGAAAAAGCAGATGTCAATGTCCCAATGGAT |
| | | CAAGTATAGGTGTGATCGCGTTGATGATGGCGGTGA |
| | | TTGGAAGCAGATCGTGATGTTCCTCCGCTATCAAGG |
| | | CGTAGAATTCATGTCATTCCTGACCGCCCTGAAACG |
| | | CTTCCTGCAGGGCATTCCTAAAAAAAATTGCATCCT |
| | | GCTGTATGGCGCGGCTAACACTGGAAAGAGTCTGTT |
| | | CGGCATGAGCCTTATGAAGTTCCTCCAGGGATCCGT |
| | | GATATGCTTTGTGAACAGCAAATCACACTTTTGGCT |
| | | TCAGCCATTGGCAGATGCAAAGATCGGCATGCTGG |
| | | ACGACGCCACAGTCCCATGCTGGAACTACATAGAC |
| | | GATAATCTCCGAAACGCATTGGACGGCAATCTGGTG |
| | | AGCATGGACGTCAAGCACAGGCCTCTGGTGCAACT |
| | | GAAGTGTCCCCCTCTCCTCATTACGTCAAACATCAA |
| | | CGCCGGAACAGATAGTCGGTGGCCGTACCTGCACA |
| | | ATAGACTTGTGGTGTTTACATTTCCTAATGAATTCCC |
| | | ATTTGACGAAAACGGCAATCCAGTATACGAGCTGA |
| | | ATGACAAGAACTGGAAGAGTTTTTTCTCTAGGACAT |
| | | GGTCCAGGTTGAGTCTCCACGAAGACGAGGATAAA |
| | | GAGAATGACGGAGACTCTTTGCCCACTTTTAAGTGC |
| | | GTGTCTGGACAAAATACCAATACCCTGTGA |
| 7 | E2 HPV16 | ATGGAGACTCTTTGCCAACGTTTAAATGTGTGTCAG |
| | nucleotide | GACAAAATACTAACACATTATGAAAATGATAGTAC |
| | sequence | AGACCTACGTGACCATATAGACTATTGGAAACACAT |
| | (natural | GCGCCTAGAATGTGCTATTTATTACAAGGCCAGAGA |
| | sequence, not | AATGGGATTTAAACATATTAACCACCAGGTGGTGCC |
| | codon- | AACACTGGCTGTATCAAAGAATAAAGCATTACAAG |
| | optimized; also | CAATTGAACTGCAACTAACGTTAGAAACAATATATA |
| | referred to herein | ACTCACAATATAGTAATGAAAAGTGGACATTACAA |
| | as E2) | GACGTTAGCCTTGAAGTGTATTTAACTGCACCAACA |
| | | GGATGTATAAAAAAACATGGATATACAGTGGAAGT |
| | | GCAGTTTGATGGAGACATATGCAATACAATGCATTA |
| | | TACAAACTGGACACATATATATATTTGTGAAGAAGC |
| | | ATCAGTAACTGTGGTAGAGGGTCAAGTTGACTATTA |
| | | TGGTTTATATTATGTTCATGAAGGAATACGAACATA |
| | | TTTTGTGCAGTTTAAAGATGATGCAGAAAAATATAG |
| | | TAAAAATAAAGTATGGGAAGTTCATGCGGGTGGTC |
| | | AGGTAATATTATGTCCTACATCTGTGTTTAGCAGCA |
| | | ACGAAGTATCCTCTCCTGAAATTATTAGGCAGCACT |
| | | TGGCCAACCACCCCGCCGCGACCCATACCAAAGCC |
| | | GTCGCCTTGGGCACCGAAGAAACACAGACGACTAT |
| | | CCAGCGACCAAGATCAGAGCCAGACACCGGAAACC |
| | | CCTGCCACACCACTAAGTTGTTGCACAGAGACTCAG |
| | | TGGACAGTGCTCCAATCCTCACTGCATTTAACAGCT |
| | | CACACAAAGGACGGATTAACTGTAATAGTAACACT |
| | | ACACCCATAGTACATTTAAAAGGTGATGCTAATACT |
| | | TTAAAATGTTTAAGATATAGATTTAAAAAGCATTGT |
| | | ACATTGTATACTGCAGTGTCGTCTACATGGCATTGG |
| | | ACAGGACATAATGTAAAACATAAAAGTGCAATTGT |
| | | TACACTTACATATGATAGTGAATGGCAACGTGACCA |
| | | ATTTTTGTCTCAAGTTAAAATACCAAAAACTATTAC |
| | | AGTGTCTACTGGATTTATGTCTATATGA |
| 8 | The E1-C | ATGTACTCCAGTGGGTCCGGCGGGGAGGGTGTTTCC |
| | (carboxy | GAAAGACACACCATCTGTCAGACCCCCCTGACTAAT |
| | terminus) | ATTCTGAACGTACTGAAAACATCCAACGCCAAGGCT |
| | sequence used in | GCCATGCTGGCGAAGTTTAAGGAGCTGTATGGCGTG |
| | Vector 22 is as | AGCTTCAGCGAACTGGTGAGACCATTCAAGAGCAA |
| | follows: | CAAGAGCACCTGTTGTGATTGGTGTATTGCCGCCTT |
| | | TGGGCTGACTCCATCCATCGCTGACTCTATTAAAAC |
| | | CCTGTTGCAACAGTACTGCCTCTACCTGCATATTCA |
| | | GTCCCTCGCTTGCTCCTGGGGAATGGTGGTGCTGCT |
| | | TCTGGTTCGGTATAAGTGTGGCAAAAACAGGGAGA |
| | | CCATCGAGAAGCTCCTTAGTAAGCTCCTGTGTGTGT |
| | | CTCCCATGTGCATGATGATTGAACCGCCAAAATTGC |
| | | GGAGCACGGCCGCCGCCCTGTACTGGTACAAAACA |
| | | GGCATAAGCAACATCAGCGAAGTGTATGGTGACAC |
| | | GCCAGAATGGATACAGAGACAGACCGTGCTCCAGC |
| | | ACAGTTTTAACGATTGCACATTTGAGCTGTCTCAGA |
| | | TGGTGCAGTGGGCTTATGATAATGACATTGTAGACG |
| | | ATTCCGAAATAGCGTATAAGTACGCCCAGCTCGCAG |
| | | ATACCAATTCCAATGCCAGCGCATTTCTGAAGTCCA |
| | | ATTCACAGGCAAAGATAGTAAAGGATTGCGCTACA |
| | | ATGTGCCGCCATTATAAAAGAGCGGAGAAAAAGCA |
| | | GATGTCAATGTCCCAATGGATCAAGTATAGGTGTGA |
| | | TCGCGTTGATGATGGCGGTGATTGGAAGCAGATCGT |
| | | GATGTTCCTCCGCTATCAAGGCGTAGAATTCATGTC |
| | | ATTCCTGACCGCCCTGAAACGCTTCCTGCAGGGCAT |
| | | TCCTAAAAAAAATTGCATCCTGCTGTATGGCGCGGC |
| | | TAACACTGGAAAGAGTCTGTTCGGCATGAGCCTTAT |
| | | GAAGTTCCTCCAGGGATCCGTGATATGCTTTGTGAA |
| | | CAGCAAATCACACTTTTGGCTTCAGCCATTGGCAGA |
| | | TGCAAAGATCGGCATGCTGGACGACGCCACAGTCC |
| | | CATGCTGGAACTACATAGACGATAATCTCCGAAAC |
| | | GCATTGGACGGCAATCTGGTGAGCATGGACGTCAA |
| | | GCACAGGCCTCTGGTGCAACTGAAGTGTCCCCCTCT |
| | | CCTCATTACGTCAAACATCAACGCCGGAACAGATA |
| | | GTCGGTGGCCGTACCTGCACAATAGACTTGTGGTGT |
| | | TTACATTTCCTAATGAATTCCCATTTGACGAAAACG |
| | | GCAATCCAGTATACGAGCTGAATGACAAGAACTGG |
| | | AAGAGTTTTTTCTCTAGGACATGGTCCAGGTTGAGT |
| | | CTCCACGAAGACGAGGATAAAGAGAATGACGGAGA |
| | | CTCTTTGCCCACTTTTAAGTGCGTGTCTGGACAAAA |
| | | TACCAATACCCTGTGA |
| 9 | E2-11 (HPV11) | ATGGAAGCCATTGCCAAAAGGCTTGATGCTTGCCAG |
| | | GATCAGCTTCTCGAGCTGTATGAGGAGAACTCTATT |
| | | GACATTCATAAACACATCATGCACTGGAAATGCATT |
| | | AGACTGGAGAGCGTGTTGCTGCACAAAGCGAAGCA |
| | | GATGGGACTGAGCCACATTGGGCTTCAGGTGGTCCC |
| | | ACCCCTTACTGTGTCAGAGACAAAGGGGCATAATG |
| | | CCATCGAGATGCAGATGCATTTGGAGTCCCTGGCGA |
| | | AAACCCAGTATGGTGTCGAGCCATGGACGCTGCAG |
| | | GACACCAGTTACGAAATGTGGCTCACCCCACCCAA |
| | | ACGCTGCTTTAAGAAGCAGGGAAATACTGTGGAGG |
| | | TAAAGTTCGATGGCTGTGAGGACAATGTTATGGAGT |
| | | ACGTGGTCTGGACACACATCTACTTGCAGGATAATG |
| | | ACTCTTGGGTAAAAGTCACTTCCTCCGTTGATGCCA |
| | | AGGGCATCTATTACACGTGTGGACAATTCAAGACGT |
| | | ACTACGTCAATTTCAATAAGGAAGCTCAGAAGTAC |
| | | GGCAGCACAAACCATTGGGAAGTTTGCTATGGCTCT |
| | | ACTGTTATTTGTTCCCCTGCTTCAGTGAGTAGCACA |
| | | GTCCGGGAAGTCAGTATAGCCGAACCCACCACTTAC |
| | | ACCCCAGCCCAGACAACCGCCCCTACAGTTTCCGCT |
| | | TGCACCACTGAGGACGGCGTGTCTGCACCTCCCCGC |
| | | AAGCGTGCAAGAGGACCCAGCACTAACAACACCCT |
| | | GTGTGTGGCCAACATACGGTCAGTGGACAGTACAA |
| | | TCAACAACATCGTAACCGACAATTACAACAAGCAC |
| | | CAGAGGCGGAATAATTGTCACTCCGCAGCAACACC |
| | | GATAGTGCAACTGCAAGGTGATAGCAACTGCCTGA |
| | | AATGCTTCCGCTATAGGCTGAATGATAAGTATAAAC |
| | | ACCTGTTTGAACTGGCATCTAGCACCTGGCATTGGG |
| | | CCTCTCCTGAAGCTCCACACAAGAACGCTATTGTGA |
| | | CACTGACTTATAGCTCCGAAGAGCAACGACAGCAA |
| | | TTTCTGAACAGCGTGAAAATCCCTCCGACCATCAGA |
| | | CATAAGGTGGGGTTTATGTCACTCCATCTCCTCTAA |
| 10 | E1-T2A-E2 | ATGGCAGACCCCGCTGGAACAAATGGAGAGGAGGG |
| | | CACTGGGTGTAACGGCTGGTTTTACGTGGAAGCAGT |
| | | CGTAGAGAAGAAGACAGGCGACGCCATTTCAGACG |
| | | ACGAGAATGAGAACGATAGCGACACTGGTGAGGAT |
| | | CTTGTGGACTTTATTGTGAACGACAATGACTATCTC |
| | | ACCCAGGCAGAAACCGAGACCGCCCACGCCCTCTT |
| | | CACAGCCCAGGAAGCTAAGCAACATCGGGATGCAG |
| | | TGCAGGTGCTCAAAAGAAAGTACCTGGTTAGTCCTC |
| | | TGTCCGACATCTCTGGATGCGTCGACAATAATATCA |
| | | GTCCAAGGCTGAAGGCTATATGCATAGAGAAGCAG |
| | | TCAAGAGCGGCGAAGAGGAGACTGTTTGAAAGCGA |
| | | GGATAGTGGATACGGGAACACAGAAGTCGAGACCC |
| | | AACAGATGCTCCAGGTGGAGGGTCGCCATGAGACT |
| | | GAGACCCCCTGCTCCCAGTACAGCGGCGGATCAGG |
| | | CGGTGGATGCTCTCAGTACTCCAGTGGGTCCGGCGG |
| | | GGAGGGTGTTTCCGAAAGACACACCATCTGTCAGA |
| | | CCCCCCTGACTAATATTCTGAACGTACTGAAAACAT |
| | | CCAACGCCAAGGCTGCCATGCTGGCGAAGTTTAAG |
| | | GAGCTGTATGGCGTGAGCTTCAGCGAACTGGTGAG |
| | | ACCATTCAAGAGCAACAAGAGCACCTGTTGTGATTG |
| | | GTGTATTGCCGCCTTTGGGCTGACTCCATCCATCGC |
| | | TGACTCTATTAAAACCCTGTTGCAACAGTACTGCCT |
| | | CTACCTGCATATTCAGTCCCTCGCTTGCTCCTGGGG |
| | | AATGGTGGTGCTGCTTCTGGTTCGGTATAAGTGTGG |
| | | CAAAAACAGGGAGACCATCGAGAAGCTCCTTAGTA |
| | | AGCTCCTGTGTGTGTCTCCCATGTGCATGATGATTG |
| | | AACCGCCAAAATTGCGGAGCACGGCCGCCGCCCTG |
| | | TACTGGTACAAAACAGGCATAAGCAACATCAGCGA |
| | | AGTGTATGGTGACACGCCAGAATGGATACAGAGAC |
| | | AGACCGTGCTCCAGCACAGTTTTAACGATTGCACAT |
| | | TTGAGCTGTCTCAGATGGTGCAGTGGGCTTATGATA |
| | | ATGACATTGTAGACGATTCCGAAATAGCGTATAAGT |
| | | ACGCCCAGCTCGCAGATACCAATTCCAATGCCAGCG |
| | | CATTTCTGAAGTCCAATTCACAGGCAAAGATAGTAA |
| | | AGGATTGCGCTACAATGTGCCGCCATTATAAAAGA |
| | | GCGGAGAAAAAGCAGATGTCAATGTCCCAATGGAT |
| | | CAAGTATAGGTGTGATCGCGTTGATGATGGCGGTGA |
| | | TTGGAAGCAGATCGTGATGTTCCTCCGCTATCAAGG |
| | | CGTAGAATTCATGTCATTCCTGACCGCCCTGAAACG |
| | | CTTCCTGCAGGGCATTCCTAAAAAAAATTGCATCCT |
| | | GCTGTATGGCGCGGCTAACACTGGAAAGAGTCTGTT |
| | | CGGCATGAGCCTTATGAAGTTCCTCCAGGGATCCGT |
| | | GATATGCTTTGTGAACAGCAAATCACACTTTTGGCT |
| | | TCAGCCATTGGCAGATGCAAAGATCGGCATGCTGG |
| | | ACGACGCCACAGTCCCATGCTGGAACTACATAGAC |
| | | GATAATCTCCGAAACGCATTGGACGGCAATCTGGTG |
| | | AGCATGGACGTCAAGCACAGGCCTCTGGTGCAACT |
| | | GAAGTGTCCCCCTCTCCTCATTACGTCAAACATCAA |
| | | CGCCGGAACAGATAGTCGGTGGCCGTACCTGCACA |
| | | ATAGACTTGTGGTGTTTACATTTCCTAATGAATTCCC |
| | | ATTTGACGAAAACGGCAATCCAGTATACGAGCTGA |
| | | ATGACAAGAACTGGAAGAGTTTTTTCTCTAGGACAT |
| | | GGTCCAGGTTGAGTCTCCACGAAGACGAGGATAAA |
| | | GAGAATGACGGAGACTCTTTGCCCACTTTTAAGTGC |
| | | GTGTCTGGACAAAATACCAATACCCTGGGAAGCGG |
| | | AGAGGGCAGAGGAAGTCTGCTAACATGCGGTGACG |
| | | TCGAGGAGAATCCTGGACCTATGGAGACTCTTTGCC |
| | | AACGTTTAAATGTGTGTCAGGACAAAATACTAACAC |
| | | ATTATGAAAATGATAGTACAGACCTACGTGACCATA |
| | | TAGACTATTGGAAACACATGCGCCTAGAATGTGCTA |
| | | TTTATTACAAGGCCAGAGAAATGGGATTTAAACATA |
| | | TTAACCACCAGGTGGTGCCAACACTGGCTGTATCAA |
| | | AGAATAAAGCATTACAAGCAATTGAACTGCAACTA |
| | | ACGTTAGAAACAATATATAACTCACAATATAGTAAT |
| | | GAAAAGTGGACATTACAAGACGTTAGCCTTGAAGT |
| | | GTATTTAACTGCACCAACAGGATGTATAAAAAAAC |
| | | ATGGATATACAGTGGAAGTGCAGTTTGATGGAGAC |
| | | ATATGCAATACAATGCATTATACAAACTGGACACAT |
| | | ATATATATTTGTGAAGAAGCATCAGTAACTGTGGTA |
| | | GAGGGTCAAGTTGACTATTATGGTTTATATTATGTT |
| | | CATGAAGGAATACGAACATATTTTGTGCAGTTTAAA |
| | | GATGATGCAGAAAAATATAGTAAAAATAAAGTATG |
| | | GGAAGTTCATGCGGGTGGTCAGGTAATATTATGTCC |
| | | TACATCTGTGTTTAGCAGCAACGAAGTATCCTCTCC |
| | | TGAAATTATTAGGCAGCACTTGGCCAACCACTCCGC |
| | | CGCGACCCATACCAAAGCCGTCGCCTTGGGCACCG |
| | | AAGAAACACAGACGACTATGCAGCGACCAAGATCA |
| | | GAGCCAGACACCGGAAACCCCTGCCACACCACTAA |
| | | GTTGTTGCACAGAGACTCAGTGGACAGTGCTCCAAT |
| | | CCTCACTGCATTTAACAGCTCACACAAAGGACGGAT |
| | | TAACTGTAATAGTAACACTACACCCATAGTACATTT |
| | | AAAAGGTGATGCTAATACTTTAAAATGTTTAAGATA |
| | | TAGATTTAAAAAGCATTGTACATTGTATACTGCAGT |
| | | GTCGTCTACATGGCATTGGACAGGACATAATGTAAA |
| | | ACATAAAAGTGCAATTGTTACACTTACATATGATAG |
| | | TGAATGGCAACGTGACCAATTTTTGTCTCAAGTTAA |
| | | AATACCAAAAACTATTACAGTGTCTACTGGATTTAT |
| | | GTCTATATGA |
| 11 | 3′LTR Fwd | CTAATTCACTCCCAACGAAG |
| | primer | |
| 12 | 5′LTR Rev | GCCGAGTCCTGCGTCGAGAG |
| 13 | Anti-HER2 | ATGTACAGGATGCAACTCCTGTCTTGCATTGCACTA |
| | antibody | AGTCTTGCACTTGTCACGGAGGTTCAGCTGGTGGAG |
| | nucleotide | TCTGGCGGTGGCCTGGTGCAGCCAGGGGGCTCACTC |
| | sequence | CGTTTGTCCTGTGCAGCTTCTGGCTTCAACATTAAA |
| | | GACACCTATATACACTGGGTGCGTCAGGCCCCGGGT |
| | | AAGGGCCTGGAATGGGTTGCAAGGATTTATCCTACG |
| | | AATGGTTATACTAGATATGCCGATAGCGTCAAGGGC |
| | | CGTTTCACTATAAGCGCAGACACATCCAAAAACAC |
| | | AGCCTACCTGCAGATGAACAGCCTGCGTGCTGAGG |
| | | ACACTGCCGTCTATTATTGTTCTAGATGGGGAGGGG |
| | | ACGGCTTCTATGCTATGGACGTGTGGGGTCAAGGAA |
| | | CCCTGGTCACCGTCTCCTCGGCTAGCACCAAGGGCC |
| | | CATCGGTCTTCCCCCTGGCACCCTCCTCCAAGAGCA |
| | | CCTCTGGGGGCACAGCGGCCCTGGGCTGCCTGGTCA |
| | | AGGACTACTTCCCCGAACCGGTGACGGTGTCGTGGA |
| | | ACTCAGGCGCCCTGACCAGCGGCGTGCACACCTTCC |
| | | CGGCTGTCCTACAGTCCTCAGGACTCTACTCCCTCA |
| | | GCAGCGTGGTGACCGTGCCCTCCAGCAGCTTGGGCA |
| | | CCCAGACCTACATCTGCAACGTGAATCACAAGCCCA |
| | | GCAACACCAAGGTGGACAAGAAAGTTGAGCCCAAA |
| | | TCTTGTGACAAAACTCACACATGCCCACCGTGCCCA |
| | | GCACCTGAACTCCTGGGGGGACCGTCAGTCTTCCTC |
| | | TTCCCCCCAAAACCCAAGGACACCCTCATGATCTCC |
| | | CGGACCCCTGAGGTCACATGCGTGGTGGTGGACGT |
| | | GAGCCACGAAGACCCTGAGGTCAAGTTCAACTGGT |
| | | ACGTGGACGGCGTGGAGGTGCATAATGCCAAGACA |
| | | AAGCCGCGGGAGGAGCAGTACAACAGCACGTACCG |
| | | TGTGGTCAGCGTCCTCACCGTCCTGCACCAGGACTG |
| | | GCTGAATGGCAAGGAGTACAAGTGCAAGGTCTCCA |
| | | ACAAAGCCCTCCCAGCCCCCATCGAGAAAACCATCT |
| | | CCAAAGCCAAAGGGCAGCCCCGAGAACCACAGGTG |
| | | TACACCCTGCCCCCATCCCGGGATGAGCTGACCAAG |
| | | AACCAGGTCAGCCTGACCTGCCTGGTCAAAGGCTTC |
| | | TATCCCAGCGACATCGCCGTGGAGTGGGAGAGCAA |
| | | TGGGCAGCCGGAGAACAACTACAAGACCACGCCTC |
| | | CCGTGCTGGACTCCGACGGCTCCTTCTTCCTCTACA |
| | | GCAAGCTCACCGTGGACAAGAGCAGGTGGCAGCAG |
| | | GGGAACGTCTTCTCATGCTCCGTGATGCATGAGGCT |
| | | CTGCACAACCACTACACGCAGAAGAGCCTCTCCCTG |
| | | TCTCCGGGTAAACGTAGACGAAAGCGCGGAAGCGG |
| | | AGAGGGCAGAGGAAGTCTGCTAACATGCGGTGACG |
| | | TCGAGGAGAATCCTGGACCTGGATCCATGTACAGG |
| | | ATGCAACTCCTGTCTTGCATTGCACTAAGTCTTGCA |
| | | CTTGTCACGGATATCCAGATGACCCAGTCCCCGAGC |
| | | TCCCTGTCCGCCTCTGTGGGCGATAGGGTCACCATC |
| | | ACCTGCCGTGCCAGTCAGGATGTGAATACTGCTGTA |
| | | GCCTGGTATCAACAGAAACCAGGAAAAGCTCCGAA |
| | | ACTACTGATTTACTCGGCATCCTTCCTCGAGTCTGG |
| | | AGTCCCTTCTCGCTTCTCTGGTTCCAGATCTGGGAC |
| | | GGATTTCACTCTGACCATCAGCAGTCTGCAGCCGGA |
| | | AGACTTCGCAACTTATTACTGTCAGCAACATTATAC |
| | | TACTCCTCCCACGTTCGGACAGGGTACCAAGGTGGA |
| | | GATCAAAGAATTCGTGGCTGCACCATCTGTCTTCAT |
| | | CTTCCCGCCATCTGATGAGCAGTTGAAATCTGGAAC |
| | | TGCCTCTGTTGTGTGCCTGCTGAATAACTTCTATCCC |
| | | AGAGAGGCCAAAGTACAGTGGAAGGTGGATAACGC |
| | | CCTCCAATCGGGTAACTCCCAGGAGAGTGTCACAG |
| | | AGCAGGACAGCAAGGACAGCACCTACAGCCTCAGC |
| | | AGCACCCTGACGCTGAGCAAAGCAGACTACGAGAA |
| | | ACACAAAGTCTACGCCTGCGAAGTCACCCATCAGG |
| | | GCCTGAGCTCGCCCGTCACAAAGAGCTTCAACAGG |
| | | GGAGAGTGTTAG |
| 14 | anti-EGFR | ATGCAGGTGCAGCTGAAGCAGAGCGGCCCGGGGCT |
| | antibody | CGTCCAGCCCTCGCAGAGCCTGAGCATCACCTGCAC |
| | nucleotide | GGTGAGCGGCTTCAGCCTGACCAACTACGGGGTGC |
| | sequence | ACTGGGTCCGGCAGTCGCCCGGCAAGGGGCTGGAG |
| | | TGGCTGGGCGTGATCTGGAGCGGCGGGAACACCGA |
| | | CTACAACACCCCCTTCACGAGCCGCCTGAGCATCAA |
| | | CAAGGACAACAGCAAGTCGCAGGTGTTCTTCAAGA |
| | | TGAACAGCCTCCAGAGCAACGACACCGCCATCTACT |
| | | ACTGCGCGCGGGCCCTGACCTACTACGACTACGAGT |
| | | TCGCCTACTGGGGCCAGGGGACCCTGGTCACGGTG |
| | | AGCGCCGCGAGCACCAAGGGCCCGAGCGTGTTCCC |
| | | CCTCGCCCCCTCCAGCAAGAGCACCAGCGGCGGGA |
| | | CCGCCGCCCTGGGCTGCCTGGTCAAGGACTACTTCC |
| | | CCGAGCCGGTGACGGTGAGCTGGAACTCGGGGGCC |
| | | CTCACCAGCGGCGTCCACACCTTCCCCGCGGTGCTG |
| | | CAGAGCAGCGGGCTGTACAGCCTCAGCTCGGTGGT |
| | | CACCGTGCCCAGCAGCAGCCTGGGCACGCAGACCT |
| | | ACATCTGCAACGTGAACCACAAGCCCAGCAACACC |
| | | AAGGTCGACAAGCGCGTGGAGCCGAAGTCGCCCAA |
| | | GAGCTGCGACAAGACCCACACGTGCCCGCCCTGCC |
| | | CCGCCCCCGAGCTGCTCGGCGGGCCCAGCGTGTTCC |
| | | TGTTCCCGCCCAAGCCCAAGGACACCCTGATGATCA |
| | | GCCGGACCCCCGAGGTCACCTGCGTGGTGGTCGAC |
| | | GTGAGCCACGAGGACCCGGAGGTGAAGTTCAACTG |
| | | GTACGTCGACGGCGTGGAGGTGCACAACGCCAAGA |
| | | CGAAGCCCCGCGAGGAGCAGTACAACAGCACCTAC |
| | | CGGGTCGTGTCGGTGCTCACCGTCCTGCACCAGGAC |
| | | TGGCTGAACGGGAAGGAGTACAAGTGCAAGGTGAG |
| | | CAACAAGGCCCTCCCCGCGCCCATCGAGAAGACCA |
| | | TCAGCAAGGCCAAGGGCCAGCCGCGCGAGCCCCAG |
| | | GTGTACACGCTGCCCCCCAGCCGGGACGAGCTGAC |
| | | CAAGAACCAGGTCAGCCTCACCTGCCTGGTGAAGG |
| | | GGTTCTACCCGTCGGACATCGCCGTGGAGTGGGAG |
| | | AGCAACGGCCAGCCCGAGAACAACTACAAGACCAC |
| | | GCCCCCGGTCCTGGACAGCGACGGCAGCTTCTTCCT |
| | | CTACAGCAAGCTGACCGTGGACAAGAGCCGCTGGC |
| | | AGCAGGGGAACGTGTTCTCGTGCAGCGTCATGCAC |
| | | GAGGCCCTGCACAACCACTACACCCAGAAGAGCCT |
| | | CAGCCTGAGCCCCGGCAAGTGAGGAAGCGGAGAGG |
| | | GCAGAGGAAGTCTGCTAACATGCGGTGACGTCGAG |
| | | GAGAATCCTGGACCTGGATCCATGGACATCCTGCTC |
| | | ACCCAGAGCCCGGTGATCCTGTCGGTCAGCCCCGGC |
| | | GAGCGGGTGAGCTTCAGCTGCCGCGCCAGCCAGTC |
| | | GATCGGGACGAACATCCACTGGTACCAGCAGCGGA |
| | | CCAACGGCAGCCCCCGCCTGCTCATCAAGTACGCGA |
| | | GCGAGAGCATCAGCGGGATTCCCTCGCGGTTCAGC |
| | | GGCAGCGGGAGCGGCACCGACTTCACCCTGAGCAT |
| | | CAACAGCGTGGAGTCGGAGGACATCGCCGACTACT |
| | | ACTGCCAGCAGAACAACAACTGGCCGACGACCTTC |
| | | GGCGCCGGGACCAAGCTGGAGCTCAAGCGCGAATT |
| | | CGTGGCTGCACCATCTGTCTTCATCTTCCCGCCATCT |
| | | GATGAGCAGTTGAAATCTGGAACTGCCTCTGTTGTG |
| | | TGCCTGCTGAATAACTTCTATCCCAGAGAGGCCAAA |
| | | GTACAGTGGAAGGTGGATAACGCCCTCCAATCGGG |
| | | TAACTCCCAGGAGAGTGTCACAGAGCAGGACAGCA |
| | | AGGACAGCACCTACAGCCTCAGCAGCACCCTGACG |
| | | CTGAGCAAAGCAGACTACGAGAAACACAAAGTCTA |
| | | CGCCTGCGAAGTCACCCATCAGGGCCTGAGCTCGCC |
| | | CGTCACAAAGAGCTTCAACAGGGGAGAGTGTTAGG |
| | | CG |
| 15 | CCR5 | GAGCAAGCTCAGTTTACA |
| | microRNA target | |
| | sequence | |
| 16 | Epstein-Barr | ATGAGGATAGCATATGCTACCCGGATACAGATTAG |
| | OriP sequence | GATAGCATATACTACCCAGATATAGATTAGGATAGC |
| | | ATATGCTACCCAGATATAGATTAGGATAGCCTATGC |
| | | TACCCAGATATAAATTAGGATAGCATATACTACCCA |
| | | GATATAGATTAGGATAGCATATGCTACCCAGATATA |
| | | GATTAGGATAGCCTATGCTACCCAGATATAGATTAG |
| | | GATAGCATATGCTACCCAGATATAGATTAGGATAGC |
| | | ATATGCTATCCAGATATTTGGGTAGTATATGCTACC |
| | | CAGATATAAATTAGGATAGCATATACTACCCTAATC |
| | | TCTATTAGGATAGCATATGCTACCCGGATACAGATT |
| | | AGGATAGCATATACTACCCAGATATAGATTAGGAT |
| | | AGCATATGCTACCCAGATATAGATTAGGATAGCCTA |
| | | TGCTACCCAGATATAAATTAGGATAGCATATACTAC |
| | | CCAGATATAGATTAGGATAGCATATGCTACCCAGAT |
| | | ATAGATTAGGATAGCCTATGCTACCCAGATATAGAT |
| | | TAGGATAGCATATGCTATCCAGATATTTGGGTAGTA |
| | | TATGCTACCCATGGCAACATTAGCCCACCGTGCTCT |
| | | CAGCGACCTCGTGAATATGAGGACCAACAACCCTG |
| | | TGCTTGGCGCTCAGGCGCAAGTGTGTGTAATTTGTC |
| | | CTCCAGATCGCAGCAATCGCGCCCCTATCTTGGCCC |
| | | GCCCACCTACTTATGCAGGTATTCCCCGGGGTGCCA |
| | | TTAGTGGTTTTGTGGGCAAGTGGTTTGACCGCAGTG |
| | | GTTAGCGGGGTTACAATCAGCCAAGTTATTACACCC |
| | | TTATTTTACAGTCCAAAACCGCAGGGCGGCGTGTGG |
| | | GGGCTGACGCGTGCCCCCACTCCACAATTTCAAAAA |
| | | AAAGAGTGGCCACTTGTCTTTGTTTATGGGCCCCAT |
| | | TGGCGTGGAGCCCCGTTTAATTTTCGGGGGTGTTAG |
| | | AGACAACCAGTGGAGTCCGCTGCTGTCGGCGTCCAC |
| | | TCTCTTTCCCCTTGTTACAAATAGAGTGTAACAACA |
| | | TGGTTCACCTGTCTTGGTCCCTGCCTGGGACACATC |
| | | TTAATAACCCCAGTATCATATTGCACTAGGATTATG |
| | | TGTTGCCCATAGCCATAAATTCGTGTGAGATGGACA |
| | | TCCAGTCTTTACGGCTTGTCCCCACCCCATGGATTTC |
| | | TATTGTTAAAGATATTCAGAATGTTTCATTCCTACA |
| | | CTAGTATTTATTGCCCAAGGGGTTTGTGAGGGTTAT |
| | | ATTGGTGTCATAGCACAATGCCACCACTGAACCCCC |
| | | CGTCCAAATTTTATTCTGGGGGCGTCACCTGAAACC |
| | | TTGTTTTCGAGCACCTCACATACACCTTACTGTTCAC |
| | | AACTCAGCAGTTATTCTATTAGCTAAACGAAGGAGA |
| | | ATGAAGAAGCAGGCGAAGATTCAGGAGAGTTCACT |
| | | GCCCGCTCCTTGATCTTCAGCCACTGCCCTTGTGACT |
| | | AAAATGGTTCACTACCCTCGTGGAATCCTGACCCCA |
| | | TGTAAATAAAACCGTGACAGCTCATGGGGTGGGAG |
| | | ATATCGCTGTTCCTTAGGACCCTTTTACTAACCCTAA |
| | | TTCGATAGCATATGCTTCCCGTTGGGTAACATATGC |
| | | TATTGAATTAGGGTTAGTCTGGATAGTATATACTAC |
| | | TACCCGGGAAGCATATGCTACCCGTTTAGGGT |
| 17 | Platelet-derived | ATGAATCGCTGCTGGGCGCTCTTCCTGTCTCTCTGCT |
| | growth factor | GCTACCTGCGTCTGGTCAGCGCCGAGGGGGACCCC |
| | (PDGF) | ATTCCCGAGGAGCTTTATGAGATGCTGAGTGACCAC |
| | | TCGATCCGCTCCTTTGATGATCTCCAACGCCTGCTG |
| | | CACGGAGACCCCGGAGAGGAAGATGGGGCCGAGTT |
| | | GGACCTGAACATGACCCGCTCCCACTCTGGAGGCG |
| | | AGCTGGAGAGCTTGGCTCGTGGAAGAAGGAGCCTG |
| | | GGTTCCCTGACCATTGCTGAGCCGGCCATGATCGCC |
| | | GAGTGCAAGACGCGCACCGAGGTGTTCGAGATCTC |
| | | CCGGCGCCTCATAGACCGCACCAACGCCAACTTCCT |
| | | GGTGTGGCCGCCCTGTGTGGAGGTGCAGCGCTGCTC |
| | | CGGCTGCTGCAACAACCGCAACGTGCAGTGCCGCC |
| | | CCACCCAGGTGCAGCTGCGACCTGTCCAGGTGAGA |
| | | AAGATCGAGATTGTGCGGAAGAAGCCAATCTTTAA |
| | | GAAGGCCACGGTGACGCTGGAAGACCACCTGGCAT |
| | | GCAAGTGTGAGACAGTGGCAGCTGCACGGCCTGTG |
| | | ACCCGAAGCCCGGGGGGTTCCCAGGAGCAGCGAGC |
| | | CAAAACGCCCCAAACTCGGGTGACCATTCGGACGG |
| | | TGCGAGTCCGCCGGCCCCCCAAGGGCAAGCACCGG |
| | | AAATTCAAGCACACGCATGACAAGACGGCACTGAA |
| | | GGAGACCCTTGGAGCCTAG |
| 18 | Bone | ATGCCCGGCGTGGCCCGCCTGCCGCTGCTGCTCGGG |
| | morphogenetic | CTGCTGCTGCTCCCGCGTCCCGGCCGGCCGCTGGAC |
| | protein 1 | TTGGCCGACTACACCTATGACCTGGCGGAGGAGGA |
| | (BMP1) | CGACTCGGAGCCCCTCAACTACAAAGACCCCTGCA |
| | nucleotide | AGGCGGCTGCCTTTCTTGGGGACATTGCCCTGGACG |
| | sequence | AAGAGGACCTGAGGGCCTTCCAGGTACAGCAGGCT |
| | (NM_001199.3) | GTGGATCTCAGACGGCACACAGCTCGTAAGTCCTCC |
| | | ATCAAAGCTGCAGTTCCAGGAAACACTTCTACCCCC |
| | | AGCTGCCAGAGCACCAACGGGCAGCCTCAGAGGGG |
| | | AGCCTGTGGGAGATGGAGAGGTAGATCCCGTAGCC |
| | | GGCGGGCGGCGACGTCCCGACCAGAGCGTGTGTGG |
| | | CCCGATGGGGTCATCCCCTTTGTCATTGGGGGAAAC |
| | | TTCACTGGTAGCCAGAGGGCAGTCTTCCGGCAGGCC |
| | | ATGAGGCACTGGGAGAAGCACACCTGTGTCACCTTC |
| | | CTGGAGCGCACTGACGAGGACAGCTATATTGTGTTC |
| | | ACCTATCGACCTTGCGGGTGCTGCTCCTACGTGGGT |
| | | CGCCGCGGCGGGGGCCCCCAGGCCATCTCCATCGG |
| | | CAAGAACTGTGACAAGTTCGGCATTGTGGTCCACGA |
| | | GCTGGGCCACGTCGTCGGCTTCTGGCACGAACACAC |
| | | TCGGCCAGACCGGGACCGCCACGTTTCCATCGTTCG |
| | | TGAGAACATCCAGCCAGGGCAGGAGTATAACTTCC |
| | | TGAAGATGGAGCCTCAGGAGGTGGAGTCCCTGGGG |
| | | GAGACCTATGACTTCGACAGCATCATGCATTACGCT |
| | | CGGAACACATTCTCCAGGGGCATCTTCCTGGATACC |
| | | ATTGTCCCCAAGTATGAGGTGAACGGGGTGAAACC |
| | | TCCCATTGGCCAAAGGACACGGCTCAGCAAGGGGG |
| | | ACATTGCCCAAGCCCGCAAGCTTTACAAGTGCCCAG |
| | | CCTGTGGAGAGACCCTGCAAGACAGCACAGGCAAC |
| | | TTCTCCTCCCCTGAATACCCCAATGGCTACTCTGCTC |
| | | ACATGCACTGCGTGTGGCGCATCTCTGTCACACCCG |
| | | GGGAGAAGATCATCCTGAACTTCACGTCCCTGGACC |
| | | TGTACCGCAGCCGCCTGTGCTGGTACGACTATGTGG |
| | | AGGTCCGAGATGGCTTCTGGAGGAAGGCGCCCCTC |
| | | CGAGGCCGCTTCTGCGGGTCCAAACTCCCTGAGCCT |
| | | ATCGTCTCCACTGACAGCCGCCTCTGGGTTGAATTC |
| | | CGCAGCAGCAGCAATTGGGTTGGAAAGGGCTTCTTT |
| | | GCAGTCTACGAAGCCATCTGCGGGGGTGATGTGAA |
| | | AAAGGACTATGGCCACATTCAATCGCCCAACTACCC |
| | | AGACGATTACCGGCCCAGCAAAGTCTGCATCTGGC |
| | | GGATCCAGGTGTCTGAGGGCTTCCACGTGGGCCTCA |
| | | CATTCCAGTCCTTTGAGATTGAGCGCCACGACAGCT |
| | | GTGCCTACGACTATCTGGAGGTGCGCGACGGGCAC |
| | | AGTGAGAGCAGCACCCTCATCGGGCGCTACTGTGG |
| | | CTATGAGAAGCCTGATGACATCAAGAGCACGTCCA |
| | | GCCGCCTCTGGCTCAAGTTCGTCTCTGACGGGTCCA |
| | | TTAACAAAGCGGGCTTTGCCGTCAACTTTTTCAAAG |
| | | AGGTGGACGAGTGCTCTCGGCCCAACCGCGGGGGC |
| | | TGTGAGCAGCGGTGCCTCAACACCCTGGGCAGCTAC |
| | | AAGTGCAGCTGTGACCCCGGGTACGAGCTGGCCCC |
| | | AGACAAGCGCCGCTGTGAGGCTGCTTGTGGCGGATT |
| | | CCTCACCAAGCTCAACGGCTCCATCACCAGCCCGGG |
| | | CTGGCCCAAGGAGTACCCCCCCAACAAGAACTGCA |
| | | TCTGGCAGCTGGTGGCCCCCACCCAGTACCGCATCT |
| | | CCCTGCAGTTTGACTTCTTTGAGACAGAGGGCAATG |
| | | ATGTGTGCAAGTACGACTTCGTGGAGGTGCGCAGTG |
| | | GACTCACAGCTGACTCCAAGCTGCATGGCAAGTTCT |
| | | GTGGTTCTGAGAAGCCCGAGGTCATCACCTCCCAGT |
| | | ACAACAACATGCGCGTGGAGTTCAAGTCCGACAAC |
| | | ACCGTGTCCAAAAAGGGCTTCAAGGCCCACTTCTTC |
| | | TCAGAAAAGAGGCCAGCTCTGCAGCCCCCTCGGGG |
| | | ACGCCCCCACCAGCTCAAATTCCGAGTGCAGAAAA |
| | | GAAACCGGACCCCCCAGTGA |
| 19 | U89348.1 | ACTACAATAATCCATGTATAAAACTAAGGGCGTAA |
| | Human | CCGAAATCGGTTGAACCGAAACCGGTTAGTATAAA |
| | papillomavirus | AGCAGACATTTTATGCACCAAAAGAGAACTGCAAT |
| | type 16 variant | GTTTCAGGACCCACAGGAGCGACCCGGAAAGTTAC |
| | nucleotide | CACAGTTATGCACAGAGCTGCAAACAACTATACAT |
| | sequence | GATATAATATTAGAATGTGTGTACTGCAAGCAACAG |
| | | TTACTGCGACGTGAGGTATATGACTTTGCTTTTCGG |
| | | GATTTATGCATAGTATATAGAGATGGGAATCCATAT |
| | | GCTGTATGTGATAAATGTTTAAAGTTTTATTCTAAA |
| | | ATTAGTGAGTATAGACATTATTGTTATAGTGTGTAT |
| | | GGAACAACATTAGAACAGCAATACAACAAACCGTT |
| | | GTGTGATTTGTTAATTAGGTGTATTAACTGTCAAAA |
| | | GCCACTGTGTCCTGAAGAAAAGCAAAGACATCTGG |
| | | ACAAAAAGCAAAGATTCCATAATATAAGGGGTCGG |
| | | TGGACCGGTCGATGTATGTCTTGTTGCAGATCATCA |
| | | AGAACACGTAGAGAAACCCAGCTGTAATCATGCAT |
| | | GGAGATACACCTACATTGCATGAATATATGTTAGAT |
| | | TTGCAACCAGAGACAACTGATCTCTACTGTTATGAG |
| | | CAATTAAATGACAGCTCAGAGGAGGAGGATGAAAT |
| | | AGATGGTCCAGCTGGACAAGCAGAACCGGACAGAG |
| | | CCCATTACAATATTGTAACCTTTTGTTGCAAGTGTG |
| | | ACTCTACGCTTCGGTTGTGCGTACAAAGCACACACG |
| | | TAGACATTCGTACTTTGGAAGACCTGTTAATGGGCA |
| | | CACTAGGAATTGTGTGCCCCATCTGTTCTCAGAAAC |
| | | CATAATCTACCATGGCTGATCCTGCAGGTACCAATG |
| | | GGGAAGAGGGTACGGGATGTAATGGATGGTTTTAT |
| | | GTAGAGGCTGTAGTGGAAAAAAAAACAGGGGATGC |
| | | TATATCAGATGACGAGAACGAAAATGACAGTGATA |
| | | CAGGTGAAGATTTGGTAGATTTTATAGTAAATGATA |
| | | ATGATTATTTAACACAGGCAGAAACAGAGACAGCA |
| | | CATGCGTTGTTTACTGCACAGGAAGCAAAACAACAT |
| | | AGAGATGCAGTACAGGTTCTAAAACGAAAGTATTT |
| | | GGGTAGTCCACTTAGTGATATTAGTGGATGTGTAGA |
| | | CAATAATATTAGTCCTAGATTAAAAGCTATATGTAT |
| | | AGAAAAACAAAGTAGAGCTGCAAAAAGGAGATTAT |
| | | TTGAAAGCAAAGACAGCGGGTATGGCAATACTGAA |
| | | GTGGAAACTCAGCAGATGTTACAGGTAGAAGGGCG |
| | | CCATGAGACTGAAACACCATGTAGTCAGTATAGTG |
| | | GTGGAAGTGGGGGTGGTTGCAGTCAGTACAGTAGT |
| | | GGAAGTGGGGGAGAGGGTGTTAGTGAAAGACACAA |
| | | TATATGCCAAACACCACTTACAAATATTTTAAATGT |
| | | ACTAAAAACTAGTAATGCAAAGGCAGCAATGTTAG |
| | | CAAAATTTAAAGAGTTATACGGGGTGAGTTTTACAG |
| | | AATTAGTAAGACCAT |
| | | TTAAAAGTAATAAATCAACGTGTTGCGATTGGTGTA |
| | | TTGCTGCATTTGGACTTACACCCAGTATAGCTGACA |
| | | GTATAAAAACACTATTACAACAATATTGTTTATATT |
| | | TACACATTCAAAGTTTAGCATGTTCATGGGGAATGG |
| | | TTGTGTTACTATTAGTAAGATATAAATGTGGAAAAA |
| | | ATAGAGAAACAATTGAAAAATTGCTGTCTAAACTAT |
| | | TATGTGTGTCTCCAATGTGTATGATGATAGAGCCTC |
| | | CAAAATTGCGTAGTACAGCAGCAGCATTATATTGGT |
| | | ATAAAACAGGTATATCAAATATTAGTGAAGTGTATG |
| | | GAGACACGCCAGAATGGATACAAAGACAAACAGTA |
| | | TTACAACATAGTTTTAATGATTGTACATTTGAATTAT |
| | | CACAGATGGTACAATGGGCCTACGATAATGACATA |
| | | GTAGACGATAGTGAAATTGCATATAAATATGCACA |
| | | ATTGGCAGACACTAATAGTAATGCAAGTGCCTTTCT |
| | | AAAAAGTAATTCACAGGCAAAAATTGTAAAGGATT |
| | | GTGCAACAATGTGTAGACATTAT |
| | | AAACGAGCAGAAAAAAAACAAATGAGTATGAGTCA |
| | | ATGGATAAAATATAGATGTGATAGGGTAGATGATG |
| | | GAGGTGATTGGAAGCAAATTGTTATGTTTTTAAGGT |
| | | ATCAAGGTGTAGAGTTTATGTCATTTTTAACTGCAT |
| | | TAAAAAGATTTTTGCAAGGCATACCTAAAAAAAATT |
| | | GCATATTACTATATGGTGCAGCTAACACAGGTAAAT |
| | | CATTATTTGGTATGAGTTTAATGAAATTTCTGCAAG |
| | | GGTCTGTAATATGTTTTGTAAATTCTAAAAGCCATT |
| | | TTTGGTTACAACCATTAGCAGATGCCAAAATAGGTA |
| | | TGTTAGATGATGCTACAGTGCCCTGTTGGAACTATA |
| | | TAGATGACAATTTAAGAAATGCATTGGATGGAAATT |
| | | TAGTTTCTATGGATGTAAAGCATAGACCATTGGTAC |
| | | AACTAAAATGCCCTCCATTATTAATTACATCTAACA |
| | | TTAATGCTGGTACAGATTCTAGGTGGCCTTATTTAC |
| | | ATAATAGATTGGTGGTGTTTACATTTCCTAATGAGT |
| | | TTCCATTTGACGAAAACGGAAA |
| | | TCCAGTGTATGAGCTTAATGATAAGAACTGGAAATC |
| | | CTTTTTCTCAAGGACGTGGTCCAGATTAAGTTTGCA |
| | | CGAGGACGAGGACAAGGAAAACGATGGAGACTCTT |
| | | TGCCAACGTTTAAATGTGTGTCAGGACAAAATACTA |
| | | ACACATTATGAAAATGATAGTACAGACCTACGTGA |
| | | CCATATAGACTATTGGAAACACATGCGCCTAGAATG |
| | | TGCTATTTATTACAAGGCCAGAGAAATGGGATTTAA |
| | | ACATATTAACCACCAGGTGGTGCCAACGCTGGCTGT |
| | | ATCAAAGAATAAAGCATTACAAGCAATTGAACTGC |
| | | AACTAACGTTAGAAACAATATATAACTCACAATATA |
| | | GTAATGAAAAGTGGACATTACAAGACGTTAGCCTT |
| | | GAAGTGTATTTAACTGCACCAACAGGATGTATAAA |
| | | AAAACATGGATATACAGTGGAAGTGCAGTTTGATG |
| | | GAGACATATGCAATACAATGCATTATACAAACTGG |
| | | ACACATATATATATTTGTGAAGAAGCATCAGTAACT |
| | | GTGGTAGAGGGTCAAGTTGACTATTATGGTTTATAT |
| | | TATGTTCATGAAGGAATACGAACATATTTTGTGCAG |
| | | TTTAAAGATGATGCAGAAAAATATAGTAAAAATAA |
| | | AGTATGGGAAGTTCATGCGGGTGGTCAGGTAATATT |
| | | ATGTCCTACATCTGTGTTTAGCAGCAACGAAGTATC |
| | | CTCTCCTGAAACTATTAGGCAGCACTTGGCCAACCA |
| | | CTCCGCCGCGACCCATACCAAA |
| | | GCCGTCGCCTTGGGCACCGAAGAAACACAGACGAC |
| | | TATCCAGCGACCAAGATCAGAGCCAGACACCGGAA |
| | | ACCCCTGCCACACCACTAAGTTGTTGCACAGAGACT |
| | | CAGTGGACAGTGCTCCAATCCTCACTGCATTTAACA |
| | | GCTCACACAAAGGACGGATTAACTGTAATAGTAAC |
| | | ACTACACCCATAGTACATTTAAAAGGTGATGCT |
| | | AATACTTTAAAATGTTTAAGATATAGATTTAAAAAG |
| | | CATTGTAAATTGTATACTGCAGTGTCGTCTACATGG |
| | | CATTGGACAGGACATAATGTAAAACATAAAAGTGC |
| | | AATTGTTACACTTACATATGATAGTGAATGGCAACG |
| | | TGACCAATTTTTGTCTCAAGTTAAAATACCAAAAAC |
| | | TATTACAGTGTCTACTGGATTTATGTCTATATGACA |
| | | AATCTTGATACTGCATACACAACATTACTGGCGTGC |
| | | TTTTTGCTTTGCTTTTGTGTGCTTTTGTGTGTCTGCCT |
| | | ATTAATACGTCCGCTGCTTTTGTCTGTGTCTACATAC |
| | | ACATCATTAATACTATTGGTATTACTATTGTGGATA |
| | | ACAGCAGCCTCTGCGTTTAGGTGTTTTATTGTATAT |
| | | ATTGTATTTGTTTATATACCATTATTTTTAATACATA |
| | | CACATGCACGCTTTTTAATTACATAATGTATATGTA |
| | | CATAATGTAATTGTTACATATAATTGTTGTATACCA |
| | | TAACTTACTATTTTTTCTTTTTTATTTTTATATATAAT |
| | | TTTTTTTTGGTTTGTTTGTTTGTTTTTTAATAAACTGT |
| | | TCTCACTTAACAATGCGACACAAACGTTCTGCAAAA |
| | | CGCACAAAACGTGCATCGGCTACCCAACTTTATAAA |
| | | ACATGCAAACAGGCAGGTACATGTCCACCTGACATT |
| | | ATACCTAAG |
| | | GTTGAAGGCAAAACTATTGCTGATCAAATATTACAA |
| | | TATGGAAGTATGGGTGTATTTTTTGGTGGGTTAGGA |
| | | ATTGGAACAGGGTCGGGTACAGGCGGACGCACTGG |
| | | GTATATTCCATTGGGAACAAGGCCTCCCACAGCTAC |
| | | AGATACACTTGCTCCTGTAAGACCCCCTTTAACAGT |
| | | AGATCCTGTGGGCCCTTCTGATCCTTCTATAGTTTCT |
| | | TTAGTGGAAGAAACTAGTTTTATTGATGCTGGTGCA |
| | | CCAACATCTGTACCTTCCATCCCCCCAGATGTATCA |
| | | GGATTTAGTATTACTACTTCAACTGATACCACACCT |
| | | GCTATATTAGATATTAATAATACTGTTACTACTGTT |
| | | ACTACACATAATAATCCCACTTTCACTGACCCATCT |
| | | GTATTGCAGCCTCCAACACCTGCAGAAACTGGAGG |
| | | GCATTTTACACTTTCATCATCCACTATTAGTACACAT |
| | | AATTATGAAGAAATTCCTATGGATACATTTATTGTT |
| | | AGCACAAACCCTAACACAGTAACTAGTAGCACACC |
| | | CATACCAGGGTCTCGCCCAGTGGCACGCCTAGGATT |
| | | ATATAGTCGCACAACACAACAAGTTAAAGTTGTAG |
| | | ACCCTGCTTTTGTAACCACTCCCACTAAACTTATTAC |
| | | ATATGATAATCCTGCATATGAAGGTATAGATGTGGA |
| | | TAATACATTATATTTTCCTAGTAATGATAATAGTATT |
| | | AATATAGCTCCAGATCCTGACTTTTTGGATATAGTT |
| | | GCTTTACATAGGCCAGCATTAACCTCTAGGCGTACT |
| | | GGCATTAGGTACAGTAGAATTGGTAATAAACAAAC |
| | | ACTACGTACTCGTAGTGGAAAATCTATAGGTGCTAA |
| | | GGTACATTATTATTATGATTTGAGTACTATTGATCCT |
| | | GCAGAAGAAA |
| | | TAGAATTACAAACTATAACACCTTCTACATATACTA |
| | | CCACTTCACATGCAGCCTCACCTACTTCTATTAATA |
| | | ATGGCTTATATGATATTTATGCAGATGACTTTATTA |
| | | CAGATACTTCTACAACCCCGGTACCATCTGTACCCT |
| | | CTACATCTTTATCAGGTTATATTCCTGCAAATACAA |
| | | CAATTCCTTTTGGTGGTGCATACAATATTCCTTTAGT |
| | | ATCAGGTCCTGATATACCCATTAATATAACTGACCA |
| | | AGCTCCTTCATTAATTCCTATAGTTCCAGGGTCTCCA |
| | | CAATATACAATTATTGCTGATGCAGGTGACTTTTAT |
| | | TTACATCCTAGTTATTACATGTTA |
| | | CGAAAACGACGTAAACGTTTACCATATTTTTTTTCA |
| | | GATGTCTCTTTGGCTGCCTAGTGAGGCCACTGTCTA |
| | | CTTGCCTCCTGTCCCAGTATCTAAGGTTGTAAGCAC |
| | | GGATGAATATGTTGCACGCACAAACATATATTATCA |
| | | TGCAGGAACATCCAGACTACTTGCAGTTGGACATCC |
| | | CTATTTTCCTATTAAAAAACCTAACAATAACAAAAT |
| | | ATTAGTTCCTAAAGTATCAGGATTACAATACAGGGT |
| | | ATTTAGAATACATTTACCTGACCCCAATAAGTTTGG |
| | | TTTTCCTGACACCTCATTTTATAATCCAGATACACA |
| | | GCGGCTGGTTTGGGCCTGTGTAGGTGTTGAGGTAGG |
| | | TCGTGGTCAGCCATTAGGTGTGGGCATTAGTGGCCA |
| | | TCCTTTATTAAATAAATTGGATGACACAGAAAATGC |
| | | TAGTGCTTATGCAGCAAATGCAGGTGTGGATAATAG |
| | | AGAATGTATATCTATGGATTACAAACAAACACAATT |
| | | GTGTTTAATTGGTTGCAAACCACCTATAGGGGAACA |
| | | CTGGGGCAAAGGATCCCCAT |
| | | GTACCAATGTTGCAGTAAATCCAGGTGATTGTCCAC |
| | | CATTAGAGTTAATAAACACAGTTATTCAGGATGGTG |
| | | ATATGGTTGATACTGGCTTTGGTGCTATGGACTTTA |
| | | CTACATTACAGGCTAACAAAAGTGAAGTTCCACTGG |
| | | ATATTTGTACATCTATTTGCAAATATCCAGATTATAT |
| | | TAAAATGGTGTCAGAACCATATGGCGACAGCTTATT |
| | | TTTTTATTTACGAAGGGAACAAATGTTTGTTAGACA |
| | | TTTATTTAATAGGGCTGGTGCTGTTGGTGAAAATGT |
| | | ACCAGACGATTTATACATTAAAGGCTCTGGGTCTAC |
| | | TGCAAATTTAGCCAGTTCAAATTATTTTCCTACACCT |
| | | AGTGGTTCTATGGTTACCTCTGATGCCCAAATATTC |
| | | AATAAACCTTATTGGTTACAACGAGCACAGGGCCA |
| | | CAATAATGGCATTTGTTGGGGTAACCAACTATTTGT |
| | | TACTGTTGTTGATACTACACGCAGTACAAATATGTC |
| | | ATTATGTGCTGCCATATCTACTTCAGAAACTACATA |
| | | TAAAAATACTAACTTTAAGGAGTACCTACGACATGG |
| | | GGAGGAATATGATTTACAGTTTATTTTTCAACTGTG |
| | | CAAAATAACCTTAACTGCAGACGTTATGACATACAT |
| | | ACATTCTATGAATTCCACTATTTTGGAGGACTGGAA |
| | | TTTTGGTCTACAACCCCCCCCAGGAGGCACACTAGA |
| | | AGATACTTATAGGTTTGTAACATCCCAGGCAATTGC |
| | | TTGTCAAAAACATACACCTCCAGCACCTAAAGAAG |
| | | ATCCCCTTAAAAAATACACTTTTTGGGAAGTAAATT |
| | | TAAAGGAAAAGTTTTCTGCAGACCTAGATCAGTTTC |
| | | CTTTAGGACGCAAATTTTTACTACAAGCAGGATTGA |
| | | AGGCCAAACCAAAATTTACATTAGGAAAACGAAAA |
| | | GCTACACCCACCACCTCATCTACCTCTACAACTGCT |
| | | AAACGCAAAAAACGTAAGCTGTAAGTATTGTATGT |
| | | ATGTTGAATTAGTGTTGTTTGTTGTTTATATGTTTGT |
| | | ATGTGCTTGTATGTGCTTGTAAATATTAAGTTGTAT |
| | | GTGTGTTTGTATGTATGGTATAATAAACACGTGTGT |
| | | ATGTGTTTTTAAATGCTTGTGTAACTATTGTGTGATG |
| | | CAACATAAATAAACTTATTGTTTCAACACCTACTAA |
| | | TTGTGTTGTGGTTATTCATTGTATATAAACTATATTT |
| | | GCTACAATCTGTTTTTGTTTTATATATACTATATTTT |
| | | GTAGCGCCAGCGGCCATTTTGTAGCTTCAACCGAAT |
| | | TCGGTTGCATGCTTTTTGGCACAAAATGTGTTTTTTT |
| | | AAATAGTTCTATGTCAGCAACTATAGTTTAAACTTG |
| | | TACGTTTCCTGCTTGCCATGCGTGCCAAATCCCTGTT |
| | | TTCCTGACCTGCACTGCTTGCCAACCATTCCATTGTT |
| | | TTTTACACTGCACTATGTGCAACTACTGAATCACTA |
| | | TGTACATTGTGTCATATAAAATAAATCACTATGCGC |
| | | CAACGCCTTACATACCGCTGTTAGGCACATATTTTT |
| | | GGCTTGTTTTAACTAACCTAATTGCATATTTGGCAT |
| | | AAGGTTTAAACTTCTAAGGCCAACTAAATGTCACCC |
| | | TAGTTCATACATGAACTGTGTAAAGGTTAGTCATAC |
| | | ATTGTTCATTTGTAAAACTGCACATGGGTGTGTGCA |
| | | AACCGTTTTGGGTTACACATTTACAAGCAACTTATA |
| | | TAATAATACTAA |
| 20 | GFP Fwd | GGATCCGCCACCATGGAGAGCGACGAGAGCGGC |
| | primer: | |
| 21 | GFP Rev primer: | GAATTCTTAGCGAGATCCGGTGGAGCC |
| 22 | Psi packaging | TACGCCAAAAATTTTGACTAGCGGAGGCTAGAAGG |
| | element | AGAGAG |
| 23 | Rev response | AGGAGCTTTGTTCCTTGGGTTCTTGGGAGCAGCAGG |
| | element | AAGCACTATGGGCGCAGCCTCAATGACGCTGACGG |
| | | TACAGGCCAGACAATTATTGTCTGGTATAGTGCAGC |
| | | AGCAGAACAATTTGCTGAGGGCTATTGAGGCGCAA |
| | | CAGCATCTGTTGCAACTCACAGTCTGGGGCATCAAG |
| | | CAGCTCCAGGCAAGAATCCTGGCTGTGGAAAGATA |
| | | CCTAAAGGATCAACAGCTCC |
| 24 | cPPT nucleotide | TTTTAAAAGAAAAGGGGGGATTGGGGGGTACAGTG |
| | sequence | CAGGGGAAAGAATAGTAGACATAATAGCAACAGAC |
| | | ATACAAACTAAAGAATTACAAAAACAAATTACAAA |
| | | ATTCAAAATTTTA |
| 25 | WPRE | AATCAACCTCTGATTACAAAATTTGTGAAAGATTGA |
| | nucleotide | CTGGTATTCTTAACTATGTTGCTCCTTTTACGCTATG |
| | sequence | TGGATACGCTGCTTTAATGCCTTTGTATCATGCTATT |
| | | GCTTCCCGTATGGCTTTCATTTTCTCCTCCTTGTATA |
| | | AATCCTGGTTGCTGTCTCTTTATGAGGAGTTGTGGC |
| | | CCGTTGTCAGGCAACGTGGCGTGGTGTGCACTGTGT |
| | | TTGCTGACGCAACCCCCACTGGTTGGGGCATTGCCA |
| | | CCACCTGTCAGCTCCTTTCCGGGACTTTCGCTTTCCC |
| | | CCTCCCTATTGCCACGGCGGAACTCATCGCCGCCTG |
| | | CCTTGCCCGCTGCTGGACAGGGGCTCGGCTGTTGGG |
| | | CACTGACAATTCCGTGGTGTTGTCGGGGAAATCATC |
| | | GTCCTTTCCTTGGCTGCTCGCCTGTGTTGCCACCTGG |
| | | ATTCTGCGCGGGACGTCCTTCTGCTACGTCCCTTCG |
| | | GCCCTCAATCCAGCGGACCTTCCTTCCCGCGGCCTG |
| | | CTGCCGGCTCTGCGGCCTCTTCCGCGTCTTCGCCTTC |
| | | GCCCTCAGACGAGTCGGATCTCCCTTTGGGCCGCCT |
| | | CCCCGCCT |
| 26 | VEGF | ATGACGGACAGACAGACAGACACCGCCCCCAGCCC |
| | nucleotide | CAGCTACCACCTCCTCCCCGGCCGGCGGCGGACAGT |
| | sequence | GGACGCGGCGGCGAGCCGCGGGCAGGGGCCGGAGC |
| | | CCGCGCCCGGAGGCGGGGTGGAGGGGGTCGGGGCT |
| | | CGCGGCGTCGCACTGAAACTTTTCGTCCAACTTCTG |
| | | GGCTGTTCTCGCTTCGGAGGAGCCGTGGTCCGCGCG |
| | | GGGGAAGCCGAGCCGAGCGGAGCCGCGAGAAGTGC |
| | | TAGCTCGGGCCGGGAGGAGCCGCAGCCGGAGGAGG |
| | | GGGAGGAGGAAGAAGAGAAGGAAGAGGAGAGGGG |
| | | GCCGCAGTGGCGACTCGGCGCTCGGAAGCCGGGCT |
| | | CATGGACGGGTGAGGCGGCGGTGTGCGCAGACAGT |
| | | GCTCCAGCCGCGCGCGCTCCCCAGGCCCTGGCCCGG |
| | | GCCTCGGGCCGGGGAGGAAGAGTAGCTCGCCGAGG |
| | | CGCCGAGGAGAGCGGGCCGCCCCACAGCCCGAGCC |
| | | GGAGAGGGAGCGCGAGCCGCGCCGGCCCCGGTCGG |
| | | GCCTCCGAAACCATGAACTTTCTGCTGTCTTGGGTG |
| | | CATTGGAGCCTTGCCTTGCTGCTCTACCTCCACCAT |
| | | GCCAAGTGGTCCCAGGCTGCACCCATGGCAGAAGG |
| | | AGGAGGGCAGAATCATCACGAAGTGGTGAAGTTCA |
| | | TGGATGTCTATCAGCGCAGCTACTGCCATCCAATCG |
| | | AGACCCTGGTGGACATCTTCCAGGAGTACCCTGATG |
| | | AGATCGAGTACATCTTCAAGCCATCCTGTGTGCCCC |
| | | TGATGCGATGCGGGGGCTGCTGCAATGACGAGGGC |
| | | CTGGAGTGTGTGCCCACTGAGGAGTCCAACATCACC |
| | | ATGCAGATTATGCGGATCAAACCTCACCAAGGCCA |
| | | GCACATAGGAGAGATGAGCTTCCTACAGCACAACA |
| | | AATGTGAATGCAGACCAAAGAAAGATAGAGCAAGA |
| | | CAAGAAAAAAAATCAGTTCGAGGAAAGGGAAAGG |
| | | GGCAAAAACGAAAGCGCAAGAAATCCCGGTATAAG |
| | | TCCTGGAGCGTGTACGTTGGTGCCCGCTGCTGTCTA |
| | | ATGCCCTGGAGCCTCCCTGGCCCCCATCCCTGTGGG |
| | | CCTTGCTCAGAGCGGAGAAAGCATTTGTTTGTACAA |
| | | GATCCGCAGACGTGTAAATGTTCCTGCAAAAACAC |
| | | AGACTCGCGTTGCAAGGCGAGGCAGCTTGAGTTAA |
| | | ACGAACGTACTTGCAGATGTGACAAGCCGAGGCGG |
| | | TGA |
| 27 | 5′ LTR | GGTCTCTCTGGTTAGACCAGATCTGAGCCTGGGAGC |
| | nucleotide | TCTCTGGCTAACTAGGGAACCCACTGCTTAAGCCTC |
| | sequence | AATAAAGCTTGCCTTGAGTGCTTCAAGTAGTGTGTG |
| | | CCCGTCTGTTGTGTGACTCTGGTAACTAGAGATCCC |
| | | TCAGACCCTTTTAGTCAGTGTGGAAAATCTCTAGCA |
| 28 | 3′ LTR | TGGAAGGGCTAATTCACTCCCAACGAAGATAAGAT |
| | nucleotide | CTGCTTTTTGCTTGTACTGGGTCTCTCTGGTTAGACC |
| | sequence | AGATCTGAGCCTGGGAGCTCTCTGGCTAACTAGGGA |
| | | ACCCACTGCTTAAGCCTCAATAAAGCTTGCCTTGAG |
| | | TGCTTCAAGTAGTGTGTGCCCGTCTGTTGTGTGACT |
| | | CTGGTAACTAGAGATCCCTCAGACCCTTTTAGTCAG |
| | | TGTGGAAAATCTCTAGCAGTAGTAGTTCATGTCA |
| 29 | CMV promoter | ACTAGTATTATGCCCAGTACATGACCTTATGGGACT |
| | nucleotide | TTCCTACTTGGCAGTACATCTACGTATTAGTCATCG |
| | sequence | CTATTACCATGGTGATGCGGTTTTGGCAGTACATCA |
| | | ATGGGCGTGGATAGCGGTTTGACTCACGGGGATTTC |
| | | CAAGTCTCCACCCCATTGACGTCAATGGGAGTTTGT |
| | | TTTGGCACCAAAATCAACGGGACTTTCCAAAATGTC |
| | | GTAACAACTCCGCCCCATTGACGCAAATGGGCGGT |
| | | AGGCGTGTACGGTGGGAGGTTTATATAAGCAGAGC |
| | | TCGTTTAGTGAACCGTCAGATCGCCTGGAGACGCCA |
| | | TCCACGCTGTTTTGACCTCCATAGAAGA |
| 30 | UbiC promoter | GCGCCGGGTTTTGGCGCCTCCCGCGGGCGCCCCCCT |
| | nucleotide | CCTCACGGCGAGCGCTGCCACGTCAGACGAAGGGC |
| | sequence | GCAGGAGCGTTCCTGATCCTTCCGCCCGGACGCTCA |
| | | GGACAGCGGCCCGCTGCTCATAAGACTCGGCCTTAG |
| | | AACCCCAGTATCAGCAGAAGGACATTTTAGGACGG |
| | | GACTTGGGTGACTCTAGGGCACTGGTTTTCTTTCCA |
| | | GAGAGCGGAACAGGCGAGGAAAAGTAGTCCCTTCT |
| | | CGGCGATTCTGCGGAGGGATCTCCGTGGGGCGGTG |
| | | AACGCCGATGATTATATAAGGACGCGCCGGGTGTG |
| | | GCACAGCTAGTTCCGTCGCAGCCGGGATTTGGGTCG |
| | | CGGTTCTTGTTTGTGGATCGCTGTGATCGTCACTTGG |
| | | TGAGTTGCGGGCTGCTGGGCTGGCCGGGGCTTTCGT |
| | | GGCCGCCGGGCCGCTCGGTGGGACGGAAGCGTGTG |
| | | GAGAGACCGCCAAGGGCTGTAGTCTGGGTCCGCGA |
| | | GCAAGGTTGCCCTGAACTGGGGGTTGGGGGGAGCG |
| | | CACAAAATGGCGGCTGTTCCCGAGTCTTGAATGGAA |
| | | GACGCTTGTAAGGCGGGCTGTGAGGTCGTTGAAAC |
| | | AAGGTGGGGGGCATGGTGGGCGGCAAGAACCCAAG |
| | | GTCTTGAGGCCTTCGCTAATGCGGGAAAGCTCTTAT |
| | | TCGGGTGAGATGGGCTGGGGCACCATCTGGGGACC |
| | | CTGACGTGAAGTTTGTCACTGACTGGAGAACTCGGG |
| | | TTTGTCGTCTGGTTGCGGGGGCGGCAGTTATGCGGT |
| | | GCCGTTGGGCAGTGCACCCGTACCTTTGGGAGCGCG |
| | | CGCCTCGTCGTGTCGTGACGTCACCCGTTCTGTTGG |
| | | CTTATAATGCAGGGTGGGGCCACCTGCCGGTAGGTG |
| | | TGCGGTAGGCTTTTCTCCGTCGCAGGACGCAGGGTT |
| | | CGGGCCTAGGGTAGGCTCTCCTGAATCGACAGGCG |
| | | CCGGACCTCTGGTGAGGGGAGGGATAAGTGAGGCG |
| | | TCAGTTTCTTTGGTCGGTTTTATGTACCTATCTTCTT |
| | | AAGTAGCTGAAGCTCCGGTTTTGAACTATGCGCTCG |
| | | GGGTTGGCGAGTGTGTTTTGTGAAGTTTTTTAGGCA |
| | | CCTTTTGAAATGTAATCATTTGGGTCAATATGTAAT |
| | | TTTCAGTGTTAGACTAGTAAA |
| 31 | EBV on | ATGAGGATAGCATATGCTACCCGGATACAGATTAG |
| | | GATAGCATATACTACCCAGATATAGATTAGGATAGC |
| | | ATATGCTACCCAGATATAGATTAGGATAGCCTATGC |
| | | TACCCAGATATAAATTAGGATAGCATATACTACCCA |
| | | GATATAGATTAGGATAGCATATGCTACCCAGATATA |
| | | GATTAGGATAGCCTATGCTACCCAGATATAGATTAG |
| | | GATAGCATATGCTACCCAGATATAGATTAGGATAGC |
| | | ATATGCTATCCAGATATTTGGGTAGTATATGCTACC |
| | | CAGATATAAATTAGGATAGCATATACTACCCTAATC |
| | | TCTATTAGGATAGCATATGCTACCCGGATACAGATT |
| | | AGGATAGCATATACTACCCAGATATAGATTAGGAT |
| | | AGCATATGCTACCCAGATATAGATTAGGATAGCCTA |
| | | TGCTACCCAGATATAAATTAGGATAGCATATACTAC |
| | | CCAGATATAGATTAGGATAGCATATGCTACCCAGAT |
| | | ATAGATTAGGATAGCCTATGCTACCCAGATATAGAT |
| | | TAGGATAGCATATGCTATCCAGATATTTGGGTAGTA |
| | | TATGCTACCCATGGCAACATTAGCCCACCGTGCTCT |
| | | CAGCGACCTCGTGAATATGAGGACCAACAACCCTG |
| | | TGCTTGGCGCTCAGGCGCAAGTGTGTGTAATTTGTC |
| | | CTCCAGATCGCAGCAATCGCGCCCCTATCTTGGCCC |
| | | GCCCACCTACTTATGCAGGTATTCCCCGGGGTGCCA |
| | | TTAGTGGTTTTGTGGGCAAGTGGTTTGACCGCAGTG |
| | | GTTAGCGGGGTTACAATCAGCCAAGTTATTACACCC |
| | | TTATTTTACAGTCCAAAACCGCAGGGCGGCGTGTGG |
| | | GGGCTGACGCGTGCCCCCACTCCACAATTTCAAAAA |
| | | AAAGAGTGGCCACTTGTCTTTGTTTATGGGCCCCAT |
| | | TGGCGTGGAGCCCCGTTTAATTTTCGGGGGTGTTAG |
| | | AGACAACCAGTGGAGTCCGCTGCTGTCGGCGTCCAC |
| | | TCTCTTTCCCCTTGTTACAAATAGAGTGTAACAACA |
| | | TGGTTCACCTGTCTTGGTCCCTGCCTGGGACACATC |
| | | TTAATAACCCCAGTATCATATTGCACTAGGATTATG |
| | | TGTTGCCCATAGCCATAAATTCGTGTGAGATGGACA |
| | | TCCAGTCTTTACGGCTTGTCCCCACCCCATGGATTTC |
| | | TATTGTTAAAGATATTCAGAATGTTTCATTCCTACA |
| | | CTAGTATTTATTGCCCAAGGGGTTTGTGAGGGTTAT |
| | | ATTGGTGTCATAGCACAATGCCACCACTGAACCCCC |
| | | CGTCCAAATTTTATTCTGGGGGCGTCACCTGAAACC |
| | | TTGTTTTCGAGCACCTCACATACACCTTACTGTTCAC |
| | | AACTCAGCAGTTATTCTATTAGCTAAACGAAGGAGA |
| | | ATGAAGAAGCAGGCGAAGATTCAGGAGAGTTCACT |
| | | GCCCGCTCCTTGATCTTCAGCCACTGCCCTTGTGACT |
| | | AAAATGGTTCACTACCCTCGTGGAATCCTGACCCCA |
| | | TGTAAATAAAACCGTGACAGCTCATGGGGTGGGAG |
| | | ATATCGCTGTTCCTTAGGACCCTTTTACTAACCCTAA |
| | | TTCGATAGCATATGCTTCCCGTTGGGTAACATATGC |
| | | TATTGAATTAGGGTTAGTCTGGATAGTATATACTAC |
| | | TACCCGGGAAGCATATGCTACCCGTTTAGGGT |
| 32 | EBNA-1 | ATGTCTGACGAGGGGCCAGGTACAGGACCTGGAAA |
| | | TGGCCTAGGAGAGAAGGGAGACACATCTGGACCAG |
| | | AAGGCTCCGGCGGCAGTGGACCTCAAAGAAGAGGG |
| | | GGTGATAACCATGGACGAGGACGGGGAAGAGGACG |
| | | AGGACGAGGAGGCGGAAGACCAGGAGCCCCGGGC |
| | | GGCTCAGGATCAGGGCCAAGACATAGAGATGGTGT |
| | | CCGGAGACCCCAAAAACGTCCAAGTTGCATTGGCT |
| | | GCAAAGGGACCCACGGTGGAACAGGAGCAGGAGC |
| | | AGGAGCGGGAGGGGCAGGAGCAGGAGGGGCAGGA |
| | | GCAGGAGGAGGGGCAGGAGCAGGAGGAGGGGCAG |
| | | GAGGGGCAGGAGGGGCAGGAGGGGCAGGAGCAGG |
| | | AGGAGGGGCAGGAGCAGGAGGAGGGGCAGGAGGG |
| | | GCAGGAGGGGCAGGAGCAGGAGGAGGGGCAGGAG |
| | | CAGGAGGAGGGGCAGGAGGGGCAGGAGCAGGAGG |
| | | AGGGGCAGGAGGGGCAGGAGGGGCAGGAGCAGGA |
| | | GGAGGGGCAGGAGCAGGAGGAGGGGCAGGAGGGG |
| | | CAGGAGCAGGAGGAGGGGCAGGAGGGGCAGGAGG |
| | | GGCAGGAGCAGGAGGAGGGGCAGGAGCAGGAGGG |
| | | GCAGGAGGGGCAGGAGGGGCAGGAGCAGGAGGGG |
| | | CAGGAGCAGGAGGAGGGGCAGGAGGGGCAGGAGG |
| | | GGCAGGAGCAGGAGGGGCAGGAGCAGGAGGGGCA |
| | | GGAGCAGGAGGGGCAGGAGCAGGAGGGGCAGGAG |
| | | GGGCAGGAGCAGGAGGGGCAGGAGGGGCAGGAGC |
| | | AGGAGGGGCAGGAGGGGCAGGAGCAGGAGGAGGG |
| | | GCAGGAGGGGCAGGAGCAGGAGGAGGGGCAGGAG |
| | | GGGCAGGAGCAGGAGGGGCAGGAGGGGCAGGAGC |
| | | AGGAGGGGCAGGAGGGGCAGGAGCAGGAGGGGCA |
| | | GGAGGGGCAGGAGCAGGAGGAGGGGCAGGAGCAG |
| | | GAGGGGCAGGAGCAGGAGGTGGAGGCCGGGGTCG |
| | | AGGAGGCAGTGGAGGCCGGGGTCGAGGAGGTAGTG |
| | | GAGGCCGGGGTCGAGGAGGTAGTGGAGGCCGCCGG |
| | | GGTAGAGGACGTGAAAGAGCCAGGGGGGGAAGTC |
| | | GTGAAAGAGCCAGGGGGAGAGGTCGTGGACGTGGA |
| | | GAAAAGAGGCCCAGGAGTCCCAGTAGTCAGTCATC |
| | | ATCATCCGGGTCTCCACCGCGCAGGCCCCCTCCAGG |
| | | TAGAAGGCCATTTTTCCACCCTGTAGGGGAAGCCGA |
| | | TTATTTTGAATACCACCAAGAAGGTGGCCCAGATGG |
| | | TGAGCCTGACGTGCCCCCGGGAGCGATAGAGCAGG |
| | | GCCCCGCAGATGACCCAGGAGAAGGCCCAAGCACT |
| | | GGACCCCGGGGTCAGGGTGATGGAGGCAGGCGCAA |
| | | AAAAGGAGGGTGGTTTGGAAAGCATCGTGGTCAAG |
| | | GAGGTTCCAACCCGAAATTTGAGAACATTGCAGAA |
| | | GGTTTAAGAGCTCTCCTGGCTAGGAGTCACGTAGAA |
| | | AGGACTACCGACGAAGGAACTTGGGTCGCCGGTGT |
| | | GTTCGTATATGGAGGTAGTAAGACCTCCCTTTACAA |
| | | CCTAAGGCGAGGAACTGCCCTTGCTATTCCACAATG |
| | | TCGTCTTACACCATTGAGTCGTCTCCCCTTTGGAATG |
| | | GCCCCTGGACCCGGCCCACAACCTGGCCCGCTAAG |
| | | GGAGTCCATTGTCTGTTATTTCATGGTCTTTTTACAA |
| | | ACTCATATATTTGCTGAGGTTTTGAAGGATGCGATT |
| | | AAGGACCTTGTTATGACAAAGCCCGCTCCTACCTGC |
| | | AATATCAGGGTGACTGTGTGCAGCTTTGACGATGGA |
| | | GTAGATTTGCCTCCCTGGTTTCCACCTATGGTGGAA |
| | | GGGGCTGCCGCGGAGGGTGATGACGGAGATGACGG |
| | | AGATGAAGGAGGTGATGGAGATGAGGGTGAGGAA |
| | | GGGCAGGAGTGA |

While certain preferred embodiments of the present invention have been described and specifically exemplified herein, it is not intended that the invention be limited to such embodiments. Various modifications may be made thereto without departing from the scope and spirit of the present invention.
DOCID: DOCPROPERTY DOCXDOCID DMS=IManage
Format=<<LIB>>\<<NUM>>.<<VER>> PRESERVELOCATION

1-60. (canceled)

61. A method of expressing at least one gene, gene product, shRNA, siRNA, miRNA, or other RNA of interest in a cell, the method comprising:
contacting the cell with a non-integrating viral delivery system, wherein the system comprises:
(a) a viral carrier, wherein the viral carrier is a lentiviral vector and contains a defective integrase gene;
(b) a first sequence encoding:
i. a heterologous episomal HPV16 origin of DNA replication comprising a 5′ truncated HPV16 Long Control Region (LCR),
wherein the 5′ truncated HPV16 LCR comprises:
a. an E1 binding site, two E2 binding sites and an SP1 binding site; or
b. an E1 binding site, three E2 binding sites and an SP1 binding site; or
c. at least two AP1 binding sites, an E1 binding site, three E2 binding sites and an SP1 binding site, wherein none of the AP1 binding sites are outside of the HPV16 LCR enhancer region;
or
ii. a heterologous episomal HPV16 origin of DNA replication comprising a 5′ truncated HPV16 LCR and at least one initiator protein specific for the heterologous episomal HPV16 origin of replication,
wherein the 5′ truncated HPV16 LCR comprises:
a. an E1 binding site, two E2 binding sites and an SP1 binding site; or
b. an E1 binding site, three E2 binding sites and an SP1 binding site; or
c. at least two AP1 binding sites, an E1 binding site, three E2 binding sites and an SP1 binding site, wherein none of the AP1 binding sites are outside of the HPV16 LCR enhancer region;
wherein the at least one initiator protein comprises:
E1, an E1 operative variant, E1-C, E2, an E2 operative variant,
E1-T2A-E2, EBNA-1, or an EBNA-1 operative fragment;
or
iii. a heterologous episomal HPV16 origin of DNA replication comprising a 5′ truncated HPV16 LCR,
wherein the 5′ truncated HPV16 LCR comprises:
a. at least three AP1 binding sites, an E1 binding site, four E2 binding sites and one SP1 binding site,
wherein one AP1 binding site is outside of the HPV16 LCR enhancer region;
or
iv. a heterologous episomal HPV16 origin of DNA replication comprising a 5′ truncated HPV16 LCR and at least one initiator protein specific for the heterologous episomal HPV16 origin of replication,
wherein the 5′ truncated HPV16 LCR comprises:
a. at least three AP1 binding sites, an E1 binding site, four E2 binding sites and one SP1 binding site,
wherein one AP1 binding site is outside of the HPV16 LCR enhancer region;
wherein the at least one initiator protein comprises:
E1, an E1 operative variant, E1-C, E2, an E2 operative variant,
E1-T2A-E2, EBNA-1, or an EBNA-1 operative fragment;
(c) a second sequence encoding at least one gene, gene product, shRNA, siRNA, miRNA, or other RNA of interest.

62. The method of claim 61, wherein the heterologous episomal HPV16 origin of DNA replication comprises a 5′ truncation of SEQ ID NO: 1.

63. The method of claim 61, wherein the heterologous episomal HPV16 origin of DNA replication comprises a 5′ truncation of at least t 200 nucleotides, at least 300 nucleotides, at least 400 nucleotides, at least 500 nucleotides, at least 600 nucleotides or at least 700 nucleotides of SEQ ID NO: 1.

64. The method of claim 61, wherein the heterologous episomal HPV16 origin of DNA replication has at least 80% sequence identity to SEQ ID NO:2.

65. The method of claim 61, wherein the heterologous episomal HPV16 origin of DNA replication has at least 80% sequence identity to SEQ ID NO:3.

66. The method of claim 61, wherein the heterologous episomal HPV16 origin of DNA replication has at least 80% sequence identity to SEQ ID NO:4.

67. The method of claim 61, wherein the heterologous episomal HPV16 origin of DNA replication has at least 80% sequence identity to SEQ ID NO:5.

68. The method of claim 61, wherein the first sequence encodes a heterologous episomal HPV16 origin of DNA replication comprising a 5′ truncated HPV LCR and two initiator proteins specific for the heterologous episomal HPV16 origin of replication, wherein the two initiator proteins are selected from: E1, an E1 operative variant, E1-C, E2, an E2 operative variant, E1-T2A-E2, EBNA-1, or an EBNA-1 operative fragment.

69. The method of claim 68, wherein expression of the first sequence is under the control of an inducible promoter.

70. The method of claim 61, wherein the at least one gene product comprises an antibody, an antibody fragment, or a growth factor.

71. The method of claim 61, wherein the miRNA encodes a C—C chemokine receptor type 5 (CCR5)-suppressing miRNA.

72. The method of claim 61, wherein the lentiviral vector further comprises an EF1/HTLV promoter and a Woodchuck Posttranscriptional Regulatory Element (WPRE).

73. The method of claim 61, wherein the cell is a lymphocyte, stem cell, epithelial cell, or neural cell.

74. The method of claim 73, wherein the cell is a human pluripotent stem cell.

75. The method of claim 61, wherein the cell is a human T cell.
